SEQUENCE LISTING (11 sequences)

SEQ ID NO: 1 - protein, 408 aa, Homo sapiens
MAENGDNEKMAALEAKICHQIEYYFGDFNLPRDKFLKEQIKLDEGWVPLEIMIKFNRLNRLTTDFNVIVEALSKSKAELMEISEDKTKIRRSPSKPLPEVTDEYKNDVKNRSVYIKGFPTDATLDDIKEWLEDKGQVLNIQMRRTLHKAFKGSIFVVFDSIESAKKFVETPGQKYKETDLLILFKDDYFAKKNEERKQNKVEAKLRAKQEQEAKQKLEEDAEMKSLEEKIGCLLKFSGDLDDQTCREDLHILFSNHGEIKWIDFVRGAKEGIILFKEKAKEALGKAKDANNGNLQLRNKEVTWEVLEGEVEKEALKKIIEDQQESLNKWKSKGRRFKGKGKGNKAAQPGSGKGKVQFQGKKTKFASDDEHDEHDENGATGPVKRAREETDKEEPASKQQKTENGAGDQ

SEQ ID NO: 2 - protein, 375 aa, Homo sapiens
MAENGDNEKMAALEAKICHQIEYYFGDFNLPRDKFLKEQIKLDEGWVPLEIMIKFNRLNRLTTDFNVIVEALSKSKAELMEISEDKTKIRRSPSKPLPEVTDEYKNDVKNRSVYIKGFPTDATLDDIKEWLEDKGQVLNIQMRRTLHKAFKGSIFVVFDSIESAKKFVETPGQKYKETDLLILFKDDYFAKKNEERKQNKVEAKLRAKQEQEAKQKLEEDAEMKSLEEKIGCLLKFSGDLDDQTCREDLHILFSNHGEIKWIDFVRGAKEGIILFKEKAKEALGKAKDANNGNLQLRNKEVTWEVLEGEVEKEALKKIIEDQQESLNKWKSKGRRFKGKGKGNKAAQPGSGKGKVQFQGKKTKFASDDEHDEHDE

SEQ ID NO: 3 - DNA, 1650 bp, Homo sapiens
atcttcttggagcgctttaggctggccggcggcgctgggaggtggagtcgttgctgttgctgtttgtgagcctgtggcgcggcttctgtgggccggaaccttaaagatagccgcaatggctgaaaatggtgataatgaaaagatggctgccctggaggccaaaatctgtcatcaaattgagtattattttggcgacttcaatttgccacgggacaagtttctaaaggaacagataaaactggatgaaggctgggtacctttggagataatgataaaattcaacaggttgaaccgtctaacaacagactttaatgtaattgtggaagcattgagcaaatccaaggcagaactcatggaaatcagtgaagataaaactaaaatcagaaggtctccaagcaaacccctacctgaagtgactgatgagtataaaaatgatgtaaaaaacagatctgtttatattaaaggcttcccaactgatgcaactcttgatgacataaaagaatggttagaagataaaggtcaagtactaaatattcagatgagaagaacattgcataaagcatttaagggatcaatttttgttgtgtttgatagcattgaatctgctaagaaatttgtagagacccctggccagaagtacaaagaaacagacctgctaatacttttcaaggacgattactttgccaaaaaaaatgaagaaagaaaacaaaataaagtggaagctaaattaagagctaaacaggagcaagaagcaaaacaaaagttagaagaagatgctgaaatgaaatctctagaagaaaagattggatgcttgctgaaattttcgggtgatttagatgatcagacctgtagagaagatttacacatacttttctcaaatcatggtgaaataaaatggatagacttcgtcagaggagcaaaagaggggataattctatttaaagaaaaagccaaggaagcattgggtaaagccaaagatgcaaataatggtaacctacaattaaggaacaaagaagtgacttgggaagtactagaaggagaggtggaaaaagaagcactgaagaaaataatagaagaccaacaagaatccctaaacaaatggaagtcaaaaggtcgtagatttaaaggaaaaggaaagggtaataaagctgcccagcctgggtctggtaaaggaaaagtacagtttcagggcaagaaaacgaaatttgctagtgatgatgaacatgatgaacatgatgaaaatggtgcaactggacctgtgaaaagagcaagagaagaaacagacaaagaagaacctgcatccaaacaacagaaaacagaaaatggtgctggagaccagtagtttagtaaaccaattttttattcattttaaataggttttaaacgacttttgtttgcggggcttttaaaaggaaaaccgaattaggtccacttcaatgtccacctgtgagaaaggaaaaatttttttgttgtttaacttgtctttttgttatgcaaatgagatttctttgaatgtattgttctgtttgtgttatttcagatgattcaaatatcaaaaggaagattcttccattaaattgcctttgtaatatgagaatgtattagtacaaactaactaataaaatatatactatatgaaaagagcaaaaa

SEQ ID NO: 4 - RNA, 21 nt, Artificial Sequence
  RNAi sequence
gaagcuaaauuaagagcuauu

SEQ ID NO: 5 - RNA, 21 nt, Artificial Sequence
  RNAi sequence
uagcucuuaauuuagcuucca

SEQ ID NO: 6 - protein, 5 aa, Artificial Sequence
  active site of caspase
  misc_feature (4)..(4): X is R, Q or G
QACXG

SEQ ID NO: 7 - protein, 188 aa, Homo sapiens
YFAKKNEERKQNKVEAKLRAKQEQEAKQKLEEDAEMKSLEEKIGCLLKFSGDLDDQTCREDLHILFSNHGEIKWIDFVRGAKEGIILFKEKAKEALGKAKDANNGNLQLRNKEVTWEVLEGEVEKEALKKIIEDQQESLNKWKSKGRRFKGKGKGNKAAQPGSGKGKVQFQGKKTKFASDDEHDEHDE

SEQ ID NO: 8 - protein, 30 aa, Artificial Sequence
  inhibitory La peptide
AKLRAKQEQEAKQKLEEDAEMKSLEEKIGC

SEQ ID NO: 9 - protein, 30 aa, Artificial Sequence
  inhibitory La peptide
GEVEKEALKKIIEDQQESLNKWKSKGRRFK

SEQ ID NO: 10 - DNA, 90 bp, Artificial Sequence
  DNA encoding inhibitory La peptide
gctaaattaagagctaaacaggagcaagaagcaaaacaaaagttagaagaagatgctgaaatgaaatctctagaagaaaagattggatgc

SEQ ID NO: 11 - DNA, 90 bp, Artificial Sequence
  DNA encoding inhibitory La peptide
ggagaggtggaaaaagaagcactgaagaaaataatagaagaccaacaagaatccctaaacaaatggaagtcaaaaggtcgtagatttaaa

(19) United States
(12) Patent Application Publication
(10) Pub. No.: US 2024/0041978 A1
(43) Pub. Date: Feb. 8, 2024

(54) LA PROTEIN AS A NOVEL REGULATOR OF OSTEOCLASTOGENESIS

(71) Applicant: The U.S.A., as Represented by the Secretary, Department of Health and Human Services, Bethesda, MD (US)

(72) Inventors: Leonid V. Chernomordik, North Potomic, MD (US); Evgenia Leikina, Derwood, MD (US); Jarred M. Whitlock, Kengsington, MD (US)

(73) Assignee: The U.S.A., as Represented by the Secretary, Department of Health and Human Services, Bethesda, MD (US)

(21) Appl. No.: 18/279,476

(22) PCT Filed: Mar. 3, 2022
(86) PCT No.: PCT/US2022/018639

§ 371 Date: Aug. 30, 2023

Related U.S. Application Data
(60) Provisional application No. 63/155,896, filed on Mar. 3, 2021.

(51) Int. Cl. A61K 38/17; C12N 15/86; A61P 19/10

(52) U.S. Cl. CPC A61K 38/177; C12N 15/86; A61P 19/10

(57) ABSTRACT

Methods are disclosed herein for modulating osteoclast fusion. In some embodiments, these methods include administering an effective amount of a Lupus autoantigen (La) protein, or an agent that modulates La protein expression or activity, to a subject in need thereof, thereby modulating osteoclast fusion in the subject. In some embodiments, the method increases osteoclast fusion and bone resorption. In other embodiments, the method decreases osteoclast fusion and bone resorption.

### CROSS REFERENCE TO RELATED APPLICATIONS

This claims the benefit of U.S. Provisional Application No. 63/155,896, filed Mar. 3, 2021, which is incorporated by reference herein.

### STATEMENT OF GOVERNMENT SUPPORT

This invention was made with Government support by the National Institutes of Health, National Institute of Child Health and Human Development. The United States Government has certain rights in the invention.

### FIELD OF THE DISCLOSURE

This relates to the field of osteoclast fusion, specifically to the use of an effective amount of a Lupus autoantigen (La) protein or an agent that modulates La protein expression or activity to modulate osteoclast fusion, such as to modulate bone resorption.

### BACKGROUND

Osteoclasts are multinucleated bone eaters responsible for essential, life-long skeletal remodeling, and their dysfunction is a major contributor to a number of bone diseases, including osteoporosis, fibrous dysplasia, Paget's disease and osteopetrosis. These pathologies, affecting >10 million Americans, all exhibit perturbations in osteoclast-mediated bone resorption. Multinucleated osteoclasts are formed by the successive fusion of mononucleated precursor cells. The number of nuclei per syncytial osteoclast and, thus, the number of fusion events that generated this cell, directly correlate with the cell's propensity to resorb bone, and the number of osteoclasts and/or the number of fusion events producing an osteoclast is significantly altered in many bone diseases. Despite the fundamental role of fusion in osteoclast formation and bone remodeling, there currently is a lack of understanding of how osteoclasts regulate their fusion and arrive at the “right size” to fulfil their biological function. Better understanding of how osteoclasts fuse can help elucidate the pathology that contributes to these bone diseases and offers new methods for the treatment of skeletal disorders.

### SUMMARY OF THE DISCLOSURE

Methods are disclosed herein for modulating osteoclast fusion. In some embodiments, these methods include administering an effective amount of a La protein or an agent that modulates La protein expression or activity, to a subject in need thereof, thereby modulating osteoclast fusion in the subject. Methods for modulating bone resorption are also disclosed.

In some embodiments, the method increases osteoclast fusion, and the agent that modulates La protein expression or activity is an agent that increases La protein expression or activity or is a La protein (or fragment thereof). In further embodiments, these methods include administering an effective amount of a La protein (or fragment thereof) or an agent that increases La protein expression or activity, to a subject in need thereof, thereby increasing osteoclast fusion in the subject. In additional embodiments, the subject has a disease that includes reduced bone resorption, and the method increases bone resorption. In some non-limiting examples, a La protein (or fragment thereof) is administered to the subject.

In other embodiments, the method decreases osteoclast fusion in the subject, and the agent decreases La protein expression or activity. In further embodiments, these methods include administering an effective agent that decreases La protein expression or activity, to a subject in need thereof, thereby decreasing osteoclast fusion in the subject. In additional embodiments, the subject has a disease that includes increased bone resorption. In some non-limiting examples, the agent is an inhibitory RNA or an antagonist antibody that specifically binds La. In other non-limiting examples, the agent is an inhibitory La peptide.

Pharmaceutical compositions are disclosed that include an effective amount of a La protein (or fragment thereof), or an agent that modulates La protein expression or activity (such as a nucleic acid molecule, vector or antibody), that are of use in any of the disclosed methods.

The foregoing and other features of the disclosure will become more apparent from the following detailed description of several embodiments which proceeds with reference to the accompanying figures.

### BRIEF DESCRIPTION OF THE FIGURES

FIGS. 1A-1G: Osteoclastogenic differentiation is accompanied by drastic changes in the steady-state levels and localization of La molecular species. (A) Representative images of stages of osteoclastogenic derivation of human monocytes following M-CSF (6 days) and M-CSF+RANKL (5 days), respectively. (staining=Phalloidin-Alexa488, nuclear dots=Hoechst) (B) Quantification of the number of fusion events normalized to the total number of nuclei observed over time following RANKL addition. (n=3) Each point represents an average of >7,500 nuclei scored. (C) Representative Bis-Tris PAGE separation and silver staining of whole protein lysates from the osteoclastogenic stages depicted in A. Lysates were run until the 50 KDa marker nearly ran off the 4-12% polyacrylamide gel to achieve maximal separation of proteins at this molecular weight, leading to the band of interest appearing misleadingly heavy. <denotes band of interest excised from both lanes and evaluated via mass spectrometry. (D) Representative Tris-Glycine Western blot evaluating La expression in whole protein lysates from primary human monocytes and the osteoclastogenic stages depicted in A. (anti-La antibody (α-La), Abcam 75927) (E) Representative tris-glycine Western blot evaluating the time course of La expression following RANKL addition. (α-La, Abcam 75927) (α-GAPDH loading control) (F) Representative digital immunofluorescence images of La in the M-CSF derived osteoclast precursors and at 3 days post RANKL application. (α-La, Abcam) (G) Representative digital immunofluorescence images of La in osteoclasts using La antibodies specifically recognizing low molecular weight (LMW) La (α-LMW La) and full-length (FL) La (α-FL La). Cells were fixed 4 days post RANKL addition, when both LMW and FL La are abundant.

FIGS. 2A-2I: Osteoclast formation depends on truncated La, but the function of the La domain is dispensable. (A) qPCR evaluation of SSB following siRNA treatment of human osteoclast precursors. (n=4) (P=0.0043) (B) Representative, phase contrast images of non-targeted and SSB-targeted human osteoclasts, as in a, stained for TRAP. (C) Quantification of the number of fusion events in formation of syncytia with 3+ nuclei in the experiments like the one in B. (n=3) (P=0.0306) (D) Representative immunofluorescence images of La in RAW 264.7 prior to mRANKL addition (Cntl.), 3 days post mRANKL addition and 5 days post mRANKL addition when massive, multinucleated osteoclasts like the one imaged here were routinely observed. (E) Representative tris-glycine Western blot of whole cell lysates taken from murine, RAW 264.7 treated as in D. murine RANKL (mRANKL) treated cells were enriched into mononucleated (Mono.) or multinucleated (Multi.) populations as described in the Methods. (Cyclophilin B (α-Cyclo B) loading control) (F) Topological illustrations of LMW La 1-375, “uncleavable” La D371A,D374A and “RNAΔ” La 1-375 Q20A_Y24A_D33I. (G) Quantification of the number of fusion events in syncytia with 3+ nuclei in RAW 264.7 cells transfected with empty, La 1-375 or La 1-375 Q20A_Y24A_D33I expression plasmids. (n=3) (P=0.0213 and 0.0173, respectively). (H) Representative fluorescence images of human monocyte-derived osteoclasts transfected with empty, La 1-375 or La D371A,D374A expression plasmids. (I) Quantification of the number of fusion events in syncytia with 3+ nuclei in H. (n=4) (P=0.0205 and 0.325, respectively). Statistical significance was evaluated via paired t-tests. *=P<0.05. **=P<0.001. Error bars=SEM.

FIGS. 3A-3G: La associates with membranes, traffics to the surface and controls osteoclast membrane fusion. (A) Western blots of cytosolic vs membrane associated protein fractions from human osteoclasts. (B) Representative digital immunofluorescence images comparing surface staining of α-Fish/TKS5 (osteoclast peripheral membrane protein used as a negative control) or α-La antibodies in human osteoclasts under non-permeabilized conditions (top) and DIC (bottom). (C) Representative digital immunofluorescence images comparing surface staining of isotype control or α-La antibodies in RAW 264.7 derived osteoclasts under non-permeabilized conditions. (D) Representative digital immunofluorescence images of La in forming osteoclasts 2-5 days post RANKL application (α-La, Abcam). Cells were stained for La at the described timepoints without membrane permeabilization. (E) Cartoons illustrating the stepwise process of the formation of multinucleated osteoclasts (top), and the approach for isolating membrane fusion stage from the preceding stages of osteoclast differentiation (bottom). Application of the hemifusion inhibitor lysophosphatidylcholine (LPC) following 48 hours of RANKL elicited osteoclastogenesis allows pre-fusion differentiation stages but blocks hemifusion, synchronizing cells. Removing LPC allows one to specifically probe membrane fusion between osteoclasts. (F) Quantification of human osteoclast fusion decoupled from pre-fusion stages and synchronized as depicted in E with fusion in the presence of α-La and with no antibodies added (Wash) normalized to α-RANK control. (n=3) (P=0.0086 and 0.1330, respectively) (G) The effects of α-FL La or α-LMW La application on synchronized fusion of human osteoclasts. Data normalized to those after application of isotype control (TgG). (n=2) (P=0.03 and 0.4581, respectively). (E, F) “LPC”-fusion observed without removal of LPC. Statistical significance was assessed via paired t-test. *=P<0.05. **=P<0.001. Error bars=SEM.

FIGS. 4A-4E: Recombinant La promotes osteoclast fusion. (A) Representative fluorescence images of human osteoclasts 3 days post RANKL addition without or with the overnight (end of day 2 post RANKL) addition of recombinant heat-inactivated La 1-408, La 1-408, La 1-375 or La 1-375 Q20A/Y24A/D33I. Recombinant proteins were added at ˜40 nM at the end day 2 post-RANKL addition, and cells were fixed the next morning. (B) Quantification of A. (inactivated n=2; others n=3) (P=0.1232, 0.0015, 0.0035 and 0.0491, respectively) (C) Quantification of the fraction of nuclei in fused cells that were present in syncytia of various sizes from A. (n=3). (D) Quantification of the number of fusion events with or without the addition of La 1-187 or La 188-375. Recombinant proteins were added at ˜40 nM at the end day 2 post-RANKL addition, and cells were fixed the next morning. (E) The quantification of synchronized fusion events (as illustrated in FIG. 3E) without (wash) and with addition of recombinant La species. “LPC”—indicates that the hemifusion inhibitor was left until fixation. (n=3) (P=0.001 and 0.03, respectively.) Statistical significance was evaluated via paired t-test. In (B, D, E) the data were normalized to those in control (no protein added in B, D and wash with no proteins added E). Error bars=SEM.

FIGS. 5A-5E: Interfering with La influences bone resorption by human osteoclasts. (A) Illustration depicting the use of fluoresceinamine-labeled chondroitin sulfate trapped in calcium phosphate coated plates to assay bone reportion. (B) Bone resorption in osteoclasts overexpressing uncleavable La (D371A, D374A) or truncated La (1-375) normalized to that for the osteoclasts transfected with empty plasmid. (n=3) (P=0.0564 and 0.0471) (C) Bone resorption in osteoclasts transfected with siRNA targeting La transcript normalized to that for non-targeted siRNA. (n=5) (P=0.0045) (D) Bone resorption in osteoclasts exposed to different concentrations of α-La normalized to that for the cells treated with isotype control antibodies at 7.5 μg/ml. (n=3) (P=0.0723, 0.0098 and 0.004) (E) Bone resorption in osteoclasts treated with 40 nM recombinant La 1-375 normalized to the control after adding the same amount of PBS. (n=3) Statistical significance was assessed via paired t-test. Error bars=SEM.

FIGS. 6A-6C: Osteoclast formation in human osteoblast/osteoclast precursor co-culture depends on La protein. (A) Schematic of the multi-well configuration of the human osteoblast/osteoclast co-culture system used: 3 sectors with osteoblasts (shown with dark grey) and 1 sector with osteoclast precursors (light gray). Multi-well dividers were removed following the M-CSF derivation of human monocytes, and osteoclast precursors and primary osteoblast media intermixed. (B) Representative immunofluorescence images comparing osteoclast fusion with control IgG or α-La antibodies. Arrowheads denote syncytia with ≥3 nuclei. (C) Quantification of osteoclast fusion in the co-cultures in the presence of α-La normalized to that in the presence of control IgG. (n=4) (P=0.0331) Statistical significance was assessed via paired t-test. Error bars=SEM.

FIGS. 7A-7G: α-La treatment suppresses ectopic osteoclast formation in fibrous dysplasia (FD) explants confirming that La regulates osteoclast activity in health and disease. (A) Illustration of ex vivo bone marrow culture system based on a tetracycline-inducible model of FD. (B) TRAP staining of bone marrow explants from a homozygous Gαₛ^{R201C} mouse (FD) and a wild-type littermate (WT). Explants were either cultured with M-CSF alone, or M-CSF and Doxy. (C) ELISA quantification of mRANKL produced in FD vs WT cultures following Doxy treatment. (n=4) (P=0.0005 (D) Representative images of FD explants activated by Doxy treated with isotype control or α-La antibody. (E) Quantification of the number of fusions producing osteoclast syncytia with ≥3 nuclei. (n=4) (P=0.0054) (F) Quantification of the number of osteoclast syncytia with ≥3 nuclei from D. (n=4) (P=0.0178) In B and D, arrowheads denote multinucleated osteoclasts and arrows denote fibrous cell masses developed after Doxy addition and characteristic for FD. Statistical significance was assessed via unpaired t-test in c or paired, t-tests in E and F. Error bars=SEM. (G) An illustration of La protein in the process of osteoclast formation. La carries out its canonical, ancient function in the nuclei of all eukaryotic cells as an essential, ubiquitous RNA binding protein. Without being bound by theory, La has an additional, specialized function in the formation of multinucleated osteoclasts. In osteoclastogenesis, La dissipates as circulating monocytes become osteoclast precursor cells. When osteoclast commitment is initiated by RANKL, La returns but is quickly cleaved by proteases and shuttled to the surface of fusing osteoclasts. At the surface of fusing osteoclasts, La plays a novel function as a membrane fusion manager. When osteoclasts arrive at the “right size” for their biological function, surface La dissipates and is replaced by canonical, non-cleaved La that returns to the nuclei of mature osteoclasts.

FIGS. 8A-8G: Characterization of monocyte derived osteoclasts and identification of La protein in osteoclasts. (A) qPCR evaluation of the osteoclast differentiation marker CTSK from the conditions depicted in FIG. 1a. (n=4) (P=0.0079) (B) Quantification of fluorescent bone resorption in osteoclast precursors (M-CSF) vs differentiated osteoclasts (RANKL). (n=3) (P=0.0066) (Day 5 post-RANKL) (C) Fraction of nuclei in syncytial osteoclasts of varying sizes. (n=4) (Day 5 post-RANKL) (D) Mass spectrometry data from six excised bands separated in six separate lanes ran on a single gel, as seen in FIG. 1b. Each lane represented a distinct cell lysate. Cells were collected from two healthy donors (1&2) and differentiated for six days with M-CSF and further differentiated for three days with M-CSF or M-CSF +RANKL. Cells from donor 2 were differentiated in three separate technical replicates (2a-2c). Vimentin was also detected in each sample, as it has a similar molecular weight to La, but vimentin levels were roughly equivalent in M-CSF vs RANKL samples. The table shows the total number of distinct peptide sequences identified in the protein group (Peptides) and the peptide spectrum matches (PSM). (E) qPCR evaluation of SSB (La gene) from the osteoclastogenic stages depicted in A (n=3) (P=0.027). (F) Representative tris-glycine Western blot evaluating La species predominate at 2 vs 5 days post RANKL addition. (G) Representative immunofluorescence images of La in forming osteoclasts 2-5 days post RANKL application (α-La, Abcam). Cells were stained for La at the described timepoints with membrane permeabilization. Statistical significance was evaluated via paired t-test. **=P<0.001 Error bars=SEM.

FIG. 9: Exogenous La constructs express at similar levels in human osteoclasts. ΔCq values of La signal in human osteoclasts transfected with empty, La D371A,D374A, La 1-375 and “RNAΔ” La 1-375 Q20A_Y24A_D33I mammalian expression plasmids. GAPDH was used as a housekeeping transcript control. (n=2) Error bars=SEM.

FIGS. 10A-10D: Formation of multinucleated osteoclasts depends on La cleavage. (A) Western blot demonstrating the ability of the caspase inhibitor z-VAD-fmk to block the production of cleaved La in human osteoclasts (Day 3-4 post-RANKL). (B) Representative immunofluorescence images of α-FL La staining in primary human osteoclasts during active fusion (Day 3) and when fusion is mostly complete (Day 5). (C) Representative immunofluorescence images of α-FL La staining in primary human osteoclasts during active fusion (Day 3) under control conditions (vehicle) and following inhibition of La cleavage via application of pan caspase inhibitor z-VAD-fmk. (D) Quantification of the number of fusion events in cells with 3+ nuclei from c. (n=3) (P=0.0455). Statistical significance was evaluated via paired t test. *=P<0.05. Error bars=SEM.

FIG. 11: RNAi suppression of La does not alter the steady state transcript levels of proteins implicated in osteoclastogenic differentiation or osteoclast fusion. qPCR evaluation of two osteoclast differentiation markers, NFATc1 and CTSK and the osteoclast fusion related transcripts syncytin 1 (SYN1), annexin A5 (ANXA5), S100A4 and ANO6 following the siRNA treatments described in FIG. 2A. (n=4) Statistical significance was assessed via paired t-test. (P=0.4051, 0.4679, 0.45650, 0.6172, 0.7899 and 0.1129). Error bars=SEM.

FIGS. 12A-12D: La interacts with Anx A5. (A) Immunoprecipitation of osteoclast lysates 3 days post-RANKL addition with or without DTSSP surface crosslinking. La supermolecular complexes were captured on immunomagnetic beads via mouse α-La or isotype control and complexes were blotted with rabbit antibodies raised towards the targets of interest. (B) Immunoprecipitation of osteoclast lysates with DTSSP surface crosslinking. La (left) or Anx A5 (right) supermolecular complexes were captured on immunomagnetic beads via mouse α-La or α-Anx A5, and complexes were blotted using rabbit antibodies raised towards targets of interest. (C) A cartoon illustration of the approach to identify membrane affinity by separating liposome bound proteins (Bottom Fraction) from soluble proteins (Top Fraction). (D) Quantification of recombinant La vs Anx A5 in liposome bound fractions, as illustrated in C. (n=2) Error bars=SEM.

FIGS. 13A-13B: α-La suppresses osteoclast-dependent bone loss in fibrous dysplasia (FD). (A) Representative radiographs of the hindlimbs of an FD mouse before and after 21 days of doxy. addition into foodstuffs. Numbers indicate the scores quantifying FD development for each bone that are then averaged to give the FD scores presented in b. (B) Time course of FD development following doxy. addition to foodstuffs scored in radiographs as in a. with antibody injections.

FIGS. 14A-14C: Identification of La osteoclast fusion domains and peptide inhibitors. (A) Linear illustration of La 1-375 protein motifs. Lines below the diagram represent the 12 overlapping peptides designed to represent protein motifs in La 188-375. (B) Quantification of the number of fusion events with or without the addition of La 1-187 or La 188-375. Recombinant proteins were added at −40 nM overnight between days 2-3 post-RANKL. (C) Same as A, using La 188-375 (+Ctl.) or the 12 peptides illustrated in B. Peptides 2 and 9 inhibit fusion and the formation of multinucleated osteoclasts.

FIGS. 15A-15C: La directly interacts with the osteoclast fusion machine annexin A5 (Anx A5). (A) La supramolecular protein complexes immunoprecipitated via immunomagnetic beads from osteoclast lysates 3 days post-RANKL. (B) Quantification of recombinant La vs Anx A5 enriched on the membranes of PC:PS liposomes, PC:PS liposomes with 5 mM Ca²⁺, or PC liposomes with 5 mM Ca²⁺. (C) Magnetic, streptavidin pulldown of Biotin-Anx A5. Lane 1=6×His-La alone, Lane 2=input before pulldown, and Lane 3=after pulldown and 4 washes.

FIGS. 16A-16C: siRNA suppression of sorting nexin 10 (SNX10) increases La content on the surface of osteoclast and results in hyperfusion of osteoclasts, phenotype characteristic for SNX10-deficiency linked infantile osteopetrosis. (A) Quantification of α-La surface staining following 48-hour siRNA treatment. (B) Quantification of nuclei per syncytia following 48-hour siRNA treatment. Normalized to non-targeted. +IgG/+α-La indicates 6 μg/ml antibody added. (C) Cartoon representation of how La traffics to the osteoclast surface, is removed to stop osteoclast fusion, how the loss of SNX10 in osteopetrosis disrupts La surface removal, and how inhibition of excess surface La rescues perturbed osteoclast multinucleation.

### SEQUENCE LISTING

The nucleic and amino acid sequences listed are shown using standard letter abbreviations for nucleotide bases, and three (or one) letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file [Sequence_Listing, Mar. 2, 2022, 12.7 KB], which is incorporated by reference herein. In the accompanying sequence listing:
SEQ ID NO: 1 is an exemplary amino acid sequence of a full-length human La protein, which includes the nuclear localization sequence, NLS.
SEQ ID NO: 2 is an exemplary amino acid sequence of amino acids 1-375 of human La protein, lacking the NLS.
SEQ ID NO: 3 is an exemplary nucleic acid sequence encoding a human La protein.
SEQ ID NOs: 4 and 5 are the nucleic acid sequences of exemplary antisense RNAs specific for La.
SEQ ID NO: 6 is an exemplary amino acid sequence of the active site of a caspase.
SEQ ID NO: 7 is an exemplary amino acid sequence of amino acids 188-375 of human La protein.
SEQ ID NOs: 8 and 9 are the amino acids sequences of exemplary inhibitor peptide fragments of La protein.
SEQ ID NOs: 10 and 11 are exemplary nucleic acid sequence encoding Peptide 2 and Peptide 9, respectively.

### DETAILED DESCRIPTION OF SEVERAL EMBODIMENTS

Osteoclasts are derived from circulating monocytes by the stepwise production of macrophage precursors in response to macrophage colony stimulating factor (M-CSF} and further osteogenic differentiation via receptor activator of nuclear factor kappa-B ligand (RANKL) (Feng et al., Bone Res. 2013; 1(1):11-26. Epub 2013/03/01. doi: 10.4248/BR201301003. PubMed PMID: 26273491; PMCID: PMC4472093). While exploring proteomic changes during osteoclast fusion, it was discovered that osteoclastogenesis involves Lupus autoantigen (named La protein), encoded by the SSB gene. La is an abundant and seemingly ubiquitous RNA chaperone observed almost exclusively in the nucleus of all human cell types and tissues (Wolin et al., Annu Rev Biochem. 2002; 71:375-403. Epub 2002/06/05. doi: 10.1146/annurev.biochem.71. 090501.15000). La's nuclear retention is due to a canonical nuclear localization sequence (NLS) at its C-terminus (Rosenblum et al., J Cell Biol. 1998; 143(4):887-99. Epub 1998/11/17. doi: 10.1083/jcb.143.4.887. PubMed PMID: 9817748). In a few specialized biological processes (e.g., apoptosis, viral infection, serum starvation), La has been reported to be cleaved by caspases and trafficked to the exofacial surface of the plasma membrane (PM) (Bachmann et al., Autoimmunity. 1992; 12(1):37-45. Epub 1992/01/01. doi: 10.3109/08916939209146128; Bachmann et al., Autoimmunity. 1991; 9(2):99-107. Epub 1991/01/01. doi: 10.3109/08916939109006745; Rutjes et al., Cell Death Differ. 1999; 6(10):976-86. Epub 1999/11/11. doi: 10.1038/sj.cdd.4400571. 5; Shiroki et al., J Virol. 1999; 73(3):2193-200. Epub 1999/02/11).

As disclosed herein, La is neither “ubiquitous” nor nuclear in osteoclastogenesis, but bidirectionally regulates osteoclast membrane fusion and bone resorption, so that suppressing La suppresses osteoclast membrane fusion and bone resorption and increasing La increases osteoclast membrane fusion and bone resorption. La is abundant in primary human monocytes but disappears when they are differentiated into osteoclast precursors. Following activation of osteoclastogenesis, La reappears as a lower molecular weight species detected on the surface of fusing cells. Following osteoclast fusion, low molecular weight La is degraded and replaced by full-length La species that returns to the nuclei of syncytial osteoclasts. Reduction of La, blocking its cleavage or blocking La at the osteoclast surface with specific antibodies inhibits osteoclast fusion. Addition of recombinant La 1-375, corresponding to La protein lacking NLS, thus, corresponding to the lower molecular weight, cleaved form of the human La protein, or overexpression of La 1-375 drastically promotes massive osteoclast fusion. Adding recombinant full-length La also promotes osteoclast fusion. However, overexpression of an “uncleavable” La mutant has no effect because it cannot be cleaved and reach the osteoclast surface. Perturbing osteoclast size by targeting La at the surface of osteoclasts bidirectionally regulates the propensity of primary, human osteoclasts to resorb bone.

It is disclosed herein that the use of La protein (or fragment thereof, such as SEQ ID NO: 7) or an agent that increases La protein activity or function, including nucleic acid molecules encoding La, can be used to increase osteoclast fusion in vivo and in vitro. These methods increase bone resorption in a subject in need thereof. In contrast, it is disclosed herein that agents that decrease La protein activity or function can be used to decrease osteoclast fusion in vivo and in vitro. These methods decrease bone resorption in a subject in need thereof.

I. Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context indicates otherwise. For example, the term “a protein” includes single or plural proteins and can be considered equivalent to the phrase “at least one protein.” As used herein, the term “comprises” means “includes.” Unless otherwise indicated “about” indicates within five percent. It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. To facilitate review of the various embodiments, the following explanations of terms are provided:

Administration: The introduction of a composition (such as one containing an agent that increases or decreases La activity) into a subject by a chosen route. Administration can be local or systemic. For example, if the route is intravenous, the composition is administered by introducing the composition into a vein of the subject. Similarly, if the route is intramuscular, the composition is administered by introducing the composition into a muscle of the subject. If the chosen route is oral, the composition is administered by ingesting the composition. Exemplary routes of administration of use in the methods disclosed herein include, but are not limited to, oral, injection (such as subcutaneous, intramuscular, intradermal, intraperitoneal, intraosseous, and intravenous), sublingual, rectal, transdermal (for example, topical), intranasal, vaginal, and inhalation routes. Administration can also be local, such as to the bone of a subject.

Animal: Living multi-cellular vertebrate organisms, a category that includes, for example, mammals and birds. The term mammal includes both human and non-human mammals. Similarly, the term “subject” includes both human and veterinary subjects.

Amino acid substitution: The replacement of an amino acid in a polypeptide (such as La, such as SEQ ID NO: 1 or 2) with one or more different amino acids. In the context of a protein sequence, an amino acid substitution is also referred to as a mutation.

Antibody: A polypeptide comprising at least a light chain or heavy chain immunoglobulin variable region which specifically recognizes and binds an epitope (e.g., an antigen, such as a La protein or fragment thereof). This includes intact immunoglobulins and the variants and portions of them well known in the art, such as Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (e.g., humanized murine antibodies), heteroconjugate antibodies (e.g., bispecific antibodies). See also, Pierce Catalog and Handbook, 1994-1995 (Pierce Chemical Co., Rockford, IL); Kuby, J., Immunology, 3^{rd} Ed., W.H. Freeman & Co., New York, 1997.

Typically, an immunoglobulin has a heavy and light chain. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs has been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor,” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin.

A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Humanized immunoglobulins can be constructed by means of genetic engineering (e.g., see U.S. Pat. No. 5,585,089).

An “antagonistic antibody” or “inhibitory antibody” is an antibody that interferes with any of the biological activities of its target polypeptide, such as a La protein.

Bone disease: Includes any disease, defect, or disorder which affects bone strength, function, and/or integrity, such as decreasing bone tensile strength and modulus. Examples of bone diseases include, but are not limited to, diseases of bone fragility, such as osteoporosis and genetic diseases which result in abnormal bone formation such as McCune-Albright syndrome (MAS) and osteogenesis imperfecta. Other examples of bone diseases include malignancies and/or cancers of the bone such as a sarcoma, such as osteosarcoma. Other bone diseases include Paget's disease of bone, fibrous dysplasia, rheumatoid arthritis osteomyelitis and osteopetrosis.

Bone Healing and Fracture Healing: Bone heals (fuses) in a unique way compared with other connective tissues. Rather than develop scar tissue, it has the ability to regenerate itself completely. The majority of fractures heal by secondary fracture healing and that involves a combination of intramembranous and endochondral ossification. Without being bound by theory, it is generally believed that the fracture healing sequence involves five discrete stages of healing. This includes an initial stage in which a hematoma is formed and inflammation occurs; a subsequent stage in which cartilage begins to form and angiogenesis proceeds, and then three successive stages of cartilage calcification, cartilage resorption and bone deposition, and ultimately a more chronic stage of bone remodeling. Generally, committed osteoprogenitor cells and uncommitted, undifferentiated mesenchymal stem cells contribute to the process of fracture healing. Bone that forms by intramembranous ossification is found early and further from the site of the fracture, results in the formation of a hard callus, and forms bone directly without first forming cartilage. Generally, two weeks after fracture, cell proliferation declines and hypertrophic chondrocytes become the dominant cell type in the chondroid callus. The resulting endochondral bone is formed adjacent to the fracture site.

Bone Resorption: The process by which osteoclasts break down tissue in bone and release minerals. Osteoclasts are generally present on the outer layer of bone, just beneath the periosteum. Attachment of the osteoclast to the osteon begins the process. The osteoclast then induces an in-folding of its cell membrane, forms an isolated acidified microenvironment between itself and the bone surface and secretes collagenase, cathepsin K and other enzymes important in the resorption process. High levels of calcium, magnesium, phosphate and products of collagen will be released into the extracellular fluid as the osteoclasts tunnel into the mineralized bone.

Bisphosphonate: A class of drugs that works by slowing bone loss. Structurally, bisphosphonates are chemically stable derivatives of inorganic pyrophosphate (PPi), a naturally occurring compound in which 2 phosphate groups are linked by esterification. Bisphosphonates have a very high affinity for bone mineral because they bind to hydroxyapatite crystals. Accordingly, bisphosphonate skeletal retention depends on availability of hydroxyapatite binding sites. Bisphosphonates are preferentially incorporated into sites of active bone remodeling, as commonly occurs in conditions characterized by accelerated skeletal turnover. Bisphosphonates suppress bone resorption by attaching to hydroxyapatite binding sites on the bone surface and by decreasing lifespan of osteoclasts and/or preventing the association of osteoclast precursors with bone. Bisphosphonates are reviewed, for example, in Drake et al., Mayo Clin. Proc. 83: 1032-1045, 2008. In some examples, a bisphosphonate is used in combination with the methods provided herein.

Cas9: An RNA-guided DNA endonuclease enzyme that can cut DNA. Cas9 has two active cutting sites (HNH and RuvC), one for each strand of a double helix. Catalytically inactive (deactivated) Cas9 (dCas9) is also encompassed by this disclosure. In some examples, a dCas9 includes one or more of the following point mutations: D10A, H840A, and N863A.

Cas9 nucleic acid and protein sequences are publicly available. For example, GenBank® Accession Nos. nucleotides 796693 . . . 800799 of CP012045.1 and nucleotides 1100046 . . . 1104152 of CP014139.1 disclose Cas9 nucleic acids, and GenBank® Accession Nos. AMA70685.1 and AKP81606.1 disclose Cas9 proteins. In some examples, the Cas9 is a deactivated form of Cas9 (dCas9), such as one that is nuclease deficient (e.g., those shown in GenBank® Accession Nos. AKA60242.1 and KR011748.1). In certain examples, Cas9 has at least 80% sequence identity, for example at least 85%, at least 90%, at least 95%, at least 96%, at least 97%, at least 98%, or at least 99% sequence identity to such sequences, and retains the ability to cut DNA.

Caspase: An enzyme that is that a cysteine-aspartic protease, cysteine aspartase or cysteine-dependent aspartate-directed protease. Caspases are a family of protease enzymes playing essential roles in programmed cell death. They are named caspases due to their specific cysteine protease activity, wherein a cysteine in its active site nucleophilically attacks and cleaves a target protein only after an aspartic acid residue. cDNA (complementary DNA): A piece of DNA lacking internal, non-coding segments (introns) and regulatory sequences that determine transcription. cDNA is synthesized in the laboratory by reverse transcription from messenger RNA extracted from cells.

Conservative variants: “Conservative” amino acid substitutions are those substitutions that do not substantially affect or decrease an activity of a polypeptide, for example a La protein's ability to modulate osteoclast fusion. Specific, non-limiting examples of a conservative substitution include the following examples:

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

The term conservative variation also includes the use of a substituted amino acid in place of an unsubstituted parent amino acid, provided that the polypeptide binds with the same affinity as the unsubstituted (parental) polypeptide. Non-conservative substitutions are those that reduce the ability of the polypeptide.

Consists essentially of and Consists Of: A polypeptide comprising an amino acid sequence that consists essentially of a specified amino acid sequence does not include any additional amino acid residues. However, the residues in the polypeptide can be modified to include non-peptide components, such as labels (for example, fluorescent, radioactive, or solid particle labels), sugars or lipids, and the N- or C-terminus of the polypeptide can be joined (for example, by peptide bond) to a linker for conjugation chemistry. A polypeptide that consists or consists essentially of a specified amino acid sequence can be glycosylated or have an amide modification. A polypeptide that consists of a specified amino acid sequence does not include any additional amino acid residues, nor does it include additional biological components, such as nucleic acids lipids, sugars, nor does it include labels.

The N- or C-terminus of a polypeptide can be joined (for example, by peptide bond) to heterologous amino acids, such as a peptide tag, or a cysteine (or other) residue in the context of a linker for conjugation chemistry. A polypeptide that consists of or consists essentially of a particular amino acid sequence can be linked via its N- or C-terminus, or a linker to a separate and distinct heterologous polypeptide, such as in the case of a fusion protein containing a first polypeptide consisting or a first sequence that is linked (via peptide bond) to a heterologous polypeptide consisting of a second sequence. In another example, the N- or C-terminus of a polypeptide that consists of or consists essentially of a particular amino acid sequence can be linked to a peptide linker (via peptide bond) that is further linked to one or more additional heterologous polypeptides. In a further example, the N- or C-terminus of a polypeptide that consists of or consists essentially of a particular amino acid sequence can be linked to one or more amino acid residues that facilitate further modification or manipulation of the polypeptide, such as a histidine tag.

Control: A reference standard. In some embodiments, the control is a negative control sample obtained from a healthy patient (such as one without bone disease), or a subject treated with a carrier, non-targeted nucleic acid sequences, scrambled nucleic acid/amino acid sequences or untreated cells from a healthy patient. In other embodiments, the control is a positive control sample obtained from a patient that has been treated with an active agent. In still other embodiments, the control is a historical control or standard reference value or range of values (such as a previously tested control sample, such as a group of patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

CRISPR (Clustered Regularly InterSpaced Short Palindromic Repeats)/Cas (CRISPR-associated protein) Editing System: An engineered nuclease system based on a bacterial system that is used for genome engineering. It is based in part on the adaptive immune response of many bacteria and archaea. Such methods can be used to allow genetic material to be added, removed, or altered at particular locations, for example in a target DNA or RNA sequence (such as a La nucleic acid sequence). Thus, CRISPR/Cas systems can be used for nucleic acid targeting (such as DNA or RNA), for example to detect a target DNA or RNA, modify a target DNA or RNA at any desired location, or cut the target DNA or RNA at any desired location. Thus, such methods can be used to modify expression of a La protein, for example by introducing a mutation to silene expression, such as knocking out the La gene.

In one example, the method edits DNA, such as a genome, and uses a Cas9 nuclease. Cas9 nuclease cleaves the DNA to generate blunt ends at the double-strand break at sites specified by a 20-nucleotide complementary strand sequence contained within the crRNA transcript. Thus, a CRISPR/Cas system can be engineered to create a double-strand break at a desired target in a genome of a cell, and harness the cell's endogenous mechanisms to repair the induced break by homology-directed repair (HDR) or nonhomologous end-joining (NHEJ). In another example, the method edits RNA, such as a La RNA, and uses a Cas13d nuclease. Cas13d nucleases cleave RNA (see for example WO 2019/040664).

Non-limiting examples of Cas nucleases include Cas1, Cas1B, Cas2, Cas3, Cas4, Cas5, Cas6, Cas7, Cas8, Cas9 (also known as Csn1 and Csx12), Cas10, Cas13d, Cpf1, C2c3, C2c2 and C2c1Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csx1, Csx15, Csf1, Csf2, Csf3, Csf4, homologs thereof.

Degenerate variant: A polynucleotide encoding a peptide that includes a sequence that is degenerate as a result of the genetic code. There are 20 natural amino acids, most of which are specified by more than one codon. Therefore, all degenerate nucleotide sequences are included in this disclosure as long as the amino acid sequence of the polypeptide encoded by the nucleotide sequence is unchanged.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signal for introns, maintenance of the correct gene reading frame to permit proper translation of mRNA, and stop codons. The term “control sequences” includes, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see e.g., Bitter et al., 1987, Methods in Enzymology 153, 516-544). For example, when cloning in bacterial systems, inducible promoters such as pL of bacteriophage lambda, plac, ptrp, ptac (ptrp-lac hybrid promoter) and the like can be used. In one embodiment, when cloning in mammalian cell systems, promoters derived from the genome of mammalian cells (such as the metallothionein promoter) or from mammalian viruses (such as the retrovirus long terminal repeat; the adenovirus late promoter; the vaccinia virus 7.5K promoter) can be used. Promoters produced by recombinant DNA or synthetic techniques can also be used to provide for transcription of the nucleic acid sequences.

Fibrous Dysplasia: A disorder where bone is lost due to ectopic formation of too many and too large osteoclasts that pathologically erode bone. Normal bone and bone marrow are then replaced with fibrous tissue, resulting in formation of bone that is weak and prone to expansion. As a result, most complications result from fracture, deformity, functional impairment, and pain. Fibrous dysplasia can affect one bone (monostotic), multiple (polyostotic), or all bones (panostotic) and can occur in isolation or in combination with café au lait skin macules and hyperfunctioning endocrinopathies, termed McCune-Albright syndrome. Fibrous dysplasia is a mosaic disease resulting from post-zygotic activating mutations of the GNAS locus, located at 20q13.2-q13.3, which codes for the a subunit of the Gs G-coupled protein receptor. On x-ray, fibrous dysplasia appears as bubbly lytic lesions, or a ground glass appearance in the bone.

Heterologous: Originating from a different genetic source, so that the biological components are not found together in nature. The components may be host cells, genes, or regulatory regions, such as promoters. Although the heterologous components are not found together in nature, they can function together, such as when a promoter heterologous to a gene is operably linked to the gene.

Host cells: Cells in which a vector can be propagated and its DNA expressed. The cell may be prokaryotic or eukaryotic. The cell can be mammalian, such as a human cell. The term also includes any progeny of the subject host cell. It is understood that all progeny may not be identical to the parental cell since there may be mutations that occur during replication. However, such progeny are included when the term “host cell” is used.

Inhibiting or treating a disease: Inhibiting a disease, such as, but not limited to, osteoporosis or osteopetrosis, refers to inhibiting the full development of a disease. In several examples, inhibiting a disease refers to lessening symptoms of the particular disease. “Treatment” refers to a therapeutic intervention that ameliorates a sign or symptom of a disease or pathological condition related to the disease. Treatment can be measured using success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical state. The treatment may be assessed by objective or subjective parameters; including the results of a physical examination or biological tests.

Inhibitory nucleic acid molecules: Includes inhibitory RNA and DNA molecules, such as an antisense oligonucleotide, a siRNA, a microRNA (miRNA), a shRNA or a ribozyme. Any type of antisense compound that specifically targets and regulates expression of a nucleic acid encoding La is contemplated for use. A La antisense compound is one which specifically hybridizes with and modulates expression of a La nucleic acid molecule. These compounds can be introduced as single-stranded, double-stranded, circular, branched or hairpin compounds and can contain structural elements such as internal or terminal bulges or loops. Double-stranded antisense compounds can be two strands hybridized to form double-stranded compounds or a single strand with sufficient self-complementarity to allow for hybridization and formation of a fully or partially double-stranded compound. In some examples, an antisense oligonucleotide is a single stranded antisense compound, such that when the antisense oligonucleotide hybridizes to a mRNA encoding La protein and the resulting duplex is recognized by RNaseH, resulting in cleavage of the mRNA. In some examples, a miRNA is a single-stranded RNA molecule, such as about 21-23 nucleotides in length that is at least partially complementary to an mRNA molecule that regulates gene expression through an RNAi pathway. In further examples, a shRNA is an RNA oligonucleotide that forms a tight hairpin, which is cleaved into siRNA. siRNA molecules are generally about 15-40 nucleotides in length, such as 20-25 nucleotides in length, and may have a 0 to 5 nucleotide overhang on the 3′ or 5′ end, or may be blunt ended. Generally, one strand of a siRNA is at least partially complementary to a nucleic acid molecule encoding La protein. Antisense compounds specifically targeting a La gene can be prepared by designing compounds that are complementary to a target nucleotide sequence, such as an mRNA sequence. Antisense compounds need not be 100% complementary to the nucleic acid molecule encoding La protein to specifically hybridize and regulate expression of the target. For example, the antisense compound, or antisense strand of the compound if a double-stranded compound, can be at least 75%, at least 80%, at least 85%, at least 90%, at least 95%, at least 99% or 100% complementary to a nucleic acid molecule encoding La protein (e.g., SEQ ID NO: 3). Methods of screening antisense compounds for specificity are known (see, for example, U.S. Publication No. 2003-0228689).

Isolated: An “isolated” biological component (such as a nucleic acid molecule or protein or organelle) has been substantially separated or purified away from other biological components in the cell of the organism in which the component naturally occurs, i.e., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins.

Lupus autoantigen (La) protein: In vivo, La protein is a 47 kDa polypeptide that often acts as an autoantigen in systemic lupus erythematosus and Sjogren's syndrome patients. La protein is present primarily in the nucleus of cells. In the nucleus, La protein is involved in the production of tRNAs, acting as an RNA polymerase III (RNAP III) transcription factor by binding to the U-rich 3′UTR of nascent transcripts, assisting in their folding and maturation. In the cytoplasm, La protein facilitates the translation of specific mRNAs, acting as a translation factor. As an RNA binding protein (RBP), La associates with subsets of mRNAs that contain a 5′-terminal oligopyrimidine (5′TOP) motif known to control protein synthesis. The binding of La to specific classes of RNA molecules regulates their downstream processing, protects them from endonuclease digestion, and organizes their export from the nucleus. It is disclosed herein that La protein modulates osteoclast fusion.

Exemplary La protein sequences, and nucleic acid sequences encoding these protein sequences, are disclosed herein, and are also disclosed in GENBANK® Accession Nos. NP_003133, Feb. 15, 2021, NM_003142, Feb. 15, 2021, and NM_001294145, Feb. 15, 2021, all incorporated herein by reference.

Label: A detectable compound or composition that is conjugated directly or indirectly to another molecule to facilitate detection of that molecule. Specific, non-limiting examples of labels include fluorescent tags, enzymatic linkages, and radioactive isotopes.

Mammal: This term includes both human and non-human mammals. Similarly, the term “subject” includes both human and veterinary subjects.

Modulate: To alter in a statistically significant manner. Modulation can be an increase or a decrease. One of skill in the art can identify an appropriate assay to determine a statistically significant increase or decrease in a parameter. These include, but are not limited to, a student's t-test or a paired ratio t test. Exemplary methods are provided in the Examples section.

Osteoblast: A mononucleate cell that is responsible for bone formation. Osteoblasts produce osteoid, which is composed mainly of Type I collagen. Osteoblasts are also responsible for mineralization of the osteoid matrix. Bone is a dynamic tissue that is constantly being reshaped by osteoblasts, which build bone, and osteoclasts, which resorb bone. Osteoblasts arise from osteoprogenitor cells located in the periosteum and the bone marrow. Osteoprogenitors are immature progenitor cells that express the master regulatory transcription factor Cbfa1/Runx2. Once osteoprogenitors start to differentiate into osteoblasts, they begin to express a range of markers including osterix, collagen type 1, alkaline phosphatase, osteocalcin, osteopontin, and osteonectin.

Osteoclast: A type of bone cell that removes bone tissue by removing its mineralized matrix by the process of bone resorption and degradation of the organic phase of the osteoid. Osteoclasts are formed by the fusion of cells of the monocyte-macrophage cell line. Osteoclastogenesis is composed of several steps including progenitor survival, differentiation to mono-nuclear pre-osteoclasts, fusion to multi-nuclear mature osteoclasts. Classically, osteoclast fusion includes four basic steps: (1) attraction/migration, (2) recognition, (3) cell-cell adhesion, and (4) membrane fusion. Osteoclasts are characterized by high expression of tartrate resistant acid phosphatase and cathepsin K and the process of osteoclastogenesis is evaluated by the detection of specific osteoclastogenic transcription factors (e.g., Nuclear factor of activated T-cells, cytoplasmic 1). Single osteoclasts can contain between 3 and 100 nuclei, varying in diameter between 10 and 300 μM. In humans, mature osteoclasts typically have 4-8 nuclei/cell

Osteocyte: Mature, non-dividing bone cells that are housed in their own lacunae (small cavities in the bone). Osteocytes are derived from osteoblasts, and they represent the final stage of maturation of the bone cell lineage. They are less active than osteoblasts, and although they are not responsible for a net increase in bone matrix, they are essential to the maintenance, routine turnover of the matrix, and play a vital role in the production of osteoclasts. The narrow, cytoplasmic processes of osteocytes remain attached to each other and to osteoblasts through canaliculi (small channels in the bone).

Osteomyelitis: An infection of the bone. Symptoms may include pain in a specific bone with overlying redness, fever, and weakness. The cause is usually a bacterial infection but can be a fungal infection. Osteomyelitis is accompanied by increased formation and activity of bone-resorbing osteoclasts and the bone loss.

Osteopetrosis: A disease also known as marble bone disease or Albers-Schönberg disease, that is a rare inherited disorder whereby the bones harden, becoming denser. Osteopetrosis can cause bones to dissolve and break. Although human osteopetrosis is a heterogeneous disorder encompassing different molecular lesions and a range of clinical features, all forms share a single pathogenic nexus in the osteoclast. Subjects with osteopetrosis have a deficiency of bone resorption, so too little bone is resorbed, resulting in too much bone being created. Osteopetroses have been categorized into two groups of disorders: autosomal dominant osteopetrosis and more severe often fatal autosomal recessive osteopetrosis (ARO), also known as infantile malignant osteopetrosis (see Sobacchi et al., Nat Rev Endocrinol. 9, 522-536; Penna et al. Autosomal recessive osteopetrosis: mechanisms and treatments. Dis Model Mech. 2021 May 1; 14(5): dmm048940. doi: 10.1242/dmm.048940 PMCID: PMC8188884; PMID: 33970241). ARO disorders have an incidence of 1:250,000 live births and originate from mutations in different genes that are involved in formation of osteoclasts (osteoclast-poor osteopetrosis) including loss-of-function mutations of genes encoding TNFSF11 or TNFRSF11A impairing the expression of RANKL or its receptor, RANK, respectively). Another type of osteoclast-rich osteopetrosis disorders referred to as osteoclast-rich osteopetrosis originate from defects in osteoclast function caused by mutations in the TCIRG1 or CLCN7 or OSTM1 genes that encode the V-type proton ATPase or chloride voltage-gated channel 7 or osteoclastogenesis-associated transmembrane protein 1, respectively. Most ARO cases, including 5% of ARO cases linked to mutations in SNX10, encoding the protein Sortin Nexin-10, are caused by the presence of inactive osteoclasts (“osteoclast-rich ARO”), see Sobacchi et al., supra; Penn et al., supra). Osteoclast-poor osteopetrosis is disclosed, for example, in Penna et al., supra.

Osteoporosis: A systemic skeletal disorder characterized by low bone mass, micro-architectural deterioration of bone tissue leading to bone fragility, and consequent increase in fracture risk. It is the most common reason for a broken bone among the elderly. The main consequence of osteoporosis is the increased risk of bone fractures. Osteoporotic fractures occur in situations where healthy people would not normally break a bone; they are therefore regarded as fragility fractures. Typical fragility fractures occur in the vertebral column, rib, hip and wrist. The World Health Organization (WHO defines osteoporosis as bone density 2.5 standard deviations below the bone density of a reference standard (i.e., generally a healthy young adult of about 30 years old). “Osteopenia” refers to a decrease in bone mineral density that is not as severe as osteoporosis, whether or not osteoporosis is present, as detected by a suitable diagnostic procedure, such as a radiographic technique. The WHO defines osteopenia as a bone density between 1 standard deviation and 2.5 standard deviations below the bone density of a reference standard as above.

Open reading frame (ORF): A series of nucleotide triplets (codons) coding for amino acids without any internal termination codons. These sequences are usually translatable into a protein.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence, such as a sequence that encodes a polypeptide. Generally, operably linked DNA sequences are contiguous and, where necessary to join two protein-coding regions, in the same reading frame.

Paget's Disease of Bone: A chronic bone disorder, that usually appears in the spine, pelvis, long bones of the limbs, and the skull, wherein their is excessive breakdown and regrowth of bone. As the bones in these subjects are excessively eroded by the ectopic formation of too many and much too large osteoclasts. Bone then regrow too quickly, they are bigger and softer than normal, may be misshapen and are easily fractured. These subjects can be treated with bisphosphonates and/or calcitonin, for example in combination with the methods provided herein. The initial stage of the disorder is characterized by increased bone resorption in a focal region, with an osteolytic lesion being a commonly detected abnormality upon radiological examination. The osteoclasts are larger than normal adult osteoclasts and show a higher number of nuclei. The excessive bone resorption is followed by an increase in bone formation, a stage characterized by increased number of normal appearing osteoblasts. The rapidly deposited bone, however, is structurally disorganized in appearance, being soft and porous in character, which accounts for the skeletal deformations and increased fracture risk. There are elevated levels of serum alkaline phosphatase and urinary excretions of hydroxyproline and pyridinoline, reflecting the increased rate of bone remodeling.

Nucleic Acid Molecule: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand.” Sequences on a nucleic acid sequence which are located 5′ to sequence of interest are referred to as “upstream sequences;” sequences a nucleotide sequence which are located 3′ to the sequence of interest are referred to as “downstream sequences.”

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (for example, rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Recombinant nucleic acid” refers to a nucleic acid having nucleotide sequences that are not naturally joined together, for example relative to a wild-type gene. This includes nucleic acid vectors comprising an amplified or assembled nucleic acid which can be used to transform a suitable host cell. In one example, a recombinant nucleic acid is one that has a sequence that is not naturally occurring or has a sequence that is made by an artificial combination of two otherwise separated segments of sequence. This artificial combination is often accomplished by chemical synthesis or, by the artificial manipulation of isolated segments of nucleic acids, such as by genetic engineering techniques. A host cell that includes the recombinant nucleic acid is referred to as a “recombinant host cell.” A recombinant nucleic acid may serve a non-coding function (such as a promoter, origin of replication, ribosome-binding site, etc.) as well.

A first sequence is an “antisense” with respect to a second sequence if a polynucleotide whose sequence is the first sequence specifically hybridizes with a polynucleotide whose sequence is the second sequence. Thus, the two sequences are complementary.

Pharmaceutically acceptable carriers: The pharmaceutically acceptable carriers of use are conventional. Remington's Pharmaceutical Sciences, by E. W. Martin, Mack Publishing Co., Easton, PA, 15th Edition (1975), describes compositions and formulations suitable for pharmaceutical delivery of the therapeutic agents (such as La protein, or an agent that modulates the function or activity of La protein) herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

A “therapeutically effective amount” is a quantity of a composition or a cell to achieve a desired effect in a subject being treated. For instance, this can be the amount necessary to affect osteoclast fusion. When administered to a subject, a dosage will generally be used that will achieve target tissue concentrations that has been shown to achieve an in vitro effect.

Polypeptide: Any chain of amino acids, regardless of length or post-translational modification (e.g., glycosylation or phosphorylation). With regard to polypeptides and proteins, the word “about” indicates integer amounts. Thus, in one example, a polypeptide “about” 29 amino acids in length is from 28 to 30 amino acids in length. Thus, a polypeptide “about” a specified number of residues can be one amino acid shorter or one amino acid longer than the specified number. A fusion polypeptide includes the amino acid sequence of a first polypeptide and a second different polypeptide (for example, a heterologous polypeptide), and can be synthesized as a single amino acid sequence. A recombinant polypeptide has an amino acid sequence that is not naturally occurring or that is made by two otherwise separated segments of an amino acid sequence.

Promoter: An array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription.

Purified: The polypeptides and polynucleotides disclosed herein can be purified (and/or synthesized) by any means known in the art (see, e.g., Guide to Protein Purification, ed. Deutscher, Meth. Enzymol. 185, Academic Press, San Diego, 1990; and Scopes, Protein Purification: Principles and Practice, Springer Verlag, New York, 1982). Substantial purification denotes purification from other proteins, nucleic acid molecules, or cellular components. A substantially purified protein is at least about 60%, 70%, 80%, 90%, 95%, 98% or 99% pure. Thus, in one specific, non-limiting example, a substantially purified protein is 90% free of other proteins or cellular components. Thus, the term purified does not require absolute purity; rather, it is intended as a relative term.

Similarly, a purified nucleic acid is one in which the nucleic acid is more enriched than the nucleic acid in its natural environment within a cell.

In some examples, a purified population of nucleic acids, proteins, or cells is greater than about 90%, 91%, 92%, 93%, 94%, 95%, 96%, 97%, 98%, 99% or 100% pure, or free other nucleic acids, proteins, or cells, respectively.

Receptor activator of nuclear factor kappa-B ligand (RANKL): A protein encoded by the TNFSF11 gene in humans. RANKL binds to RANK on cells of the myeloid lineage and functions in osteoclast differentiation and activation. RANKL may also bind osteoprotegerin, a protein secreted mainly by cells of the osteoblast lineage which is a potent inhibitor of osteoclast formation by preventing binding of RANKL to RANK. RANKL also has a function in the immune system, where it is expressed by T helper cells and is thought to be involved in dendritic cell maturation. It is a dendritic cell survival factor and helps regulate T cell-dependent immune responses. T cell activation induces RANKL expression and can lead to an increase of osteoclastogenesis and bone loss. Exemplary amino acid and nucleic acid sequences for human RANKL can be found in GENBANK Accession No. NM_003701.4, Feb. 16, 2021, incorporated herein by reference.

Rheumatoid arthritis: A chronic autoimmune disorder that affects the joints, wherein there is inflammation of the synovial membrane. Joints become swollen, tender and warm, and stiffness limits their movement. With time, multiple joints are affected (polyarthritis). Most commonly involved are the small joints of the hands, feet and cervical spine, but larger joints like the shoulder and knee can also be involved. Bone destruction in rheumatoid arthritis is mainly caused by increased differentiation and activity of osteoclasts.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Methods of alignment of sequences for comparison are known t. Various programs and alignment algorithms are described in: Smith and Waterman, Adv. Appl. Math. 2:482, 1981; Needleman and Wunsch, 1970, J Mol Biol 48, 443-453; Higgins and Sharp, 1988, Gene 73, 237-244; Higgins and Sharp, 1989, CABIOS 5, 151-153; Corpet et al., 1988, Nucleic Acids Research 16, 10881-10890; and Pearson and Lipman, 1988, Proc Natl Acad Sci USA 85, 2444-2448. Altschul et al., 1994, Nature Genet 6, 119-129, presents a detailed consideration of sequence alignment methods and homology calculations.

The NCBI Basic Local Alignment Search Tool (BLAST) (Altschul et al., 1990, J Mol Biol 215, 403-410) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Homologs and variants of a polypeptide are typically characterized by possession of at least 75%, for example at least 80%, sequence identity counted over the full length alignment with the amino acid sequence of a polypeptide using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and can possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Thus, in some examples, variants of a polypeptide or nucleic acid sequence are typically characterized by possession of at least about 75%, for example, at least about 80%, 85%, 90%, 91%, 92%, 93%, 94%, 95%, 96%, 97%, 98% or 99% sequence identity counted over the full length alignment with the amino acid or nucleotide sequence of interest. Sequences with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids (or 30-60 nucleotides), and may possess sequence identities of at least 85% or at least 90% or 95% depending on their similarity to the reference sequence. Methods for determining sequence identity over such short windows are available at the NCBI website on the internet.

As used herein, reference to “at least 90% identity” (or similar language) refers to “at least 90%, at least 91%, at least 92%, at least 93%, at least 94%, at least 95%, at least 96%, at least 97%, at least 98%, at least 99%, or even 100% identity” to a specified reference sequence. Thus, La protein (or fragment thereof) has at least 90% sequence identity to SEQ ID NO: 1, 2, 7, 8 or 9 is one that has at least 90%, at least 91%, at least 92%, at least 93%, at least 94%, at least 95%, at least 96%, at least 97%, at least 98%, at least 99%, or even 100% identity to SEQ ID NO: 1 or 2. Similarly, a La coding sequence that has at least 90% sequence identity to SEQ ID NO: 3 is one having at least 90%, at least 91%, at least 92%, at least 93%, at least 94%, at least 95%, at least 96%, at least 97%, at least 98%, at least 99%, or even 100% identity to SEQ ID NO: 3.

Subject: Living multi-cellular vertebrate organisms, a category that includes human and non-human mammals, such as non-human primates, rats, mice, dogs, cats, horses, cows and pigs. In an example, a subject is a human. In an additional example, a subject is selected that is in need of modulating osteoclast fusion. For example, the subject can need increased or decreased osteoclast fusion, or can need increased or decreased bone resorption.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker gene and other genetic elements known in the art. Vectors include plasmid vectors, including plasmids for expression in gram-negative and gram-positive bacterial cell. Exemplary vectors include those for expression in E. coli and Salmonella. Vectors also include viral vectors, such as, but are not limited to, retrovirus, lentiviral, adeno-associated virus (AAV), orthopox, avipox, fowlpox, capripox, suipox, adenoviral, herpes virus, alpha virus, baculovirus, Sindbis virus, vaccinia virus and poliovirus vectors. Vectors also include vectors for expression in yeast cells or mammalian cells.

Virus: Microscopic infectious organism that reproduces inside living cells. A virus consists essentially of a core of a single nucleic acid surrounded by a protein coat and has the ability to replicate only inside a living cell. “Viral replication” is the production of additional virus by the occurrence of at least one viral life cycle.

II. La Protein and Agents that Increase La Protein Function and/or Activity for Use in Increasing Osteoclast Fusion

Methods are disclosed herein for increasing osteoclast fusion. These methods can be used to increase bone resorption in a subject. These methods can utilize La protein or an agent that increase La protein expression or activity in the subject, such as a nucleic acid molecule encoding La protein. Exemplary agents of use in these methods are disclosed below.

A. La Protein

It is disclosed herein that addition of “cleaved,” recombinant La, corresponding to an amino acid sequence of La protein but lacking a carboxyterminal nuclear localization sequence, or overexpression of “cleaved” La drastically promotes osteoclast fusion. Any form of La protein is of use in the disclosed methods that functions to promote osteoclast fusion.

An exemplary human La protein sequence is provided below.

(SEQ ID NO: 1)
MAENGDNEKM AALEAKICHQ IEYYFGDFNL PRDKFLKEQI
KLDEGWVPLE IMIKFNRLNR LTTDENVIVE ALSKSKAELM
EISEDKTKIR RSPSKPLPEV TDEYKNDVKN RSVYIKGFPT
DATLDDIKEW LEDKGQVLNI QMRRTLHKAF KGSIFVVFDS
IESAKKFVET PGQKYKETDL LILFKDDYFA KKNEERKQNK
VEAKLRAKQE QEAKQKLEED AEMKSLEEKI GCLLKFSGDL
DDQTCREDLH ILFSNHGEIK WIDFVRGAKE GIILFKEKAK
EALGKAKDAN NGNLQLRNKE VTWEVLEGEV EKEALKKIIE
DQQESLNKWK SKGRRFKGKG KGNKAAQPGS GKGKVQFQGK
KTKFASDDEH DEHDENGATG PVKRAREETD KEEPASKQQK
TENGAGDQ

With reference to position number, the N-terminal amino acid is position one, and the remaining positions are the amino acids, as numbered sequentially.

In some embodiments, the La protein of use comprises or consists of amino acids 1-375 of SEQ ID NO: 1:

(SEQ ID NO: 2)
MAENGDNEKM AALEAKICHQ IEYYFGDFNL PRDKFLKEQI
KLDEGWVPLE IMIKFNRLNR LTTDENVIVE ALSKSKAELM
EISEDKTKIR RSPSKPLPEV TDEYKNDVKN RSVYIKGFPT
DATLDDIKEW LEDKGQVLNI QMRRTLHKAF KGSIFVVFDS
IESAKKFVET PGQKYKETDL LILFKDDYFA KKNEERKONK
VEAKLRAKQE QEAKQKLEED AEMKSLEEKI GCLLKFSGDL
DDQTCREDLH ILFSNHGEIK WIDFVRGAKE GIILFKEKAK
EALGKAKDAN NGNLQLRNKE VTWEVLEGEV EKEALKKIIE
DQQESLNKWK SKGRRFKGKG KGNKAAQPGS GKGKVQFQGK
KTKFASDDEHDEHDE

In more embodiments, the La protein of use comprises or consists of amino acids 188-375 of SEQ ID NO: 1:

(SEQ ID NO: 7)
YFA KKNEERKQNK VEAKLRAKQE QEAKQKLEED AEMKSLEEKI
GCLLKFSGDL DDQTCREDLH ILFSNHGEIK WIDFVRGAKE
GIILFKEKAK EALGKAKDAN NGNLQLRNKE VTWEVLEGEV
EKEALKKIIE DQQESLNKWK SKGRRFKGKG KGNKAAQPGS
GKGKVQFQGK KTKFASDDEHDEHDE.

In specific non-limiting examples, the La protein comprises, consists essentially of, or consists of the amino acid sequence of SEQ ID NO: 1, SEQ ID NO: 2, or SEQ ID NO: 7.

Any fragment or variant of La protein is of use in the methods disclosed herein, provided it functions to promote osteoclast fusion. In some embodiments, a La protein of use in the disclosed methods includes a sequence having at least 95%, at least 96%, at least 97%, at least 98%, at least 99% or 100% sequence identity to SEQ ID NO: 1, SEQ ID NO: 2, or SEQ ID NO: 7, and promotes osteoclast fusion. The La protein can include at most 15, 14, 13, 12, 11, 10, 9, 8, 7, 6, 5, 4, 3, 2 or 1 conservative substitutions in SEQ ID NO: 1, SEQ ID NO: 2, or SEQ ID NO: 7. In some examples, the La protein is SEQ ID NO: 1, SEQ ID NO: 2, or SEQ ID NO: 7 containing 15, 14, 13, 12, 11, 10, 9, 8, 7, 6, 5, 4, 3, 2 or 1 conservative substitutions. The La protein of use in the methods disclosed herein can be naturally occurring or recombinant.

In some embodiments, the La protein comprises, consists essentially of, or consists of an amino acid sequence at least 95%, at least 96%, at least 97%, at least 98%, at least 99% or 100% identical to SEQ ID NO: 2, wherein the La protein is at most 375 amino acids in length and does not include amino acids 376-408 of SEQ ID NO: 1. In more embodiments, the La protein comprises the amino acid sequence of SEQ ID NO: 2, is at least 95% identical to SEQ ID NO: 1, such that substitutions are present only in amino acids 376-408 of SEQ ID NO: 2. In some embodiments, the substitutions are conservative substitutions.

In more embodiments, the La protein comprises, consists essentially of, or consists of an amino acid sequence at least 95%, at least 96%, at least 97%, at least 98%, at least 99% or 100% identical to SEQ ID NO: 7, wherein the La protein does not include amino acids 1-187 and 376-408 of SEQ ID NO: 1. In more embodiments, the La protein comprises the amino acid sequence of SEQ ID NO: 7, and is at least 95% identical to SEQ ID NO: 1, such that substitutions are present only in amino acids 1-187 and 376-408 of SEQ ID NO: 1. In some embodiments, the substitutions are conservative substitutions.

In further embodiments, the La protein comprises, consists essentially of, or consists of amino acids 300-375 of SEQ ID NO: 1; and/or amino acids 6-242 of SEQ ID NO: 1, wherein the La protein does not comprise amino acids 376-408 of SEQ ID NO: 1. In more embodiments, La protein comprises, consists essentially of, or consists of amino acids 300-375 of SEQ ID NO: 1; and/or amino acids 6-242 of SEQ ID NO: 1, and includes substitutions in amino acids 376-408 of SEQ ID NO: 1. In some embodiments, the substitutions are conservative substitutions.

La protein fragments are also of use in the disclosed methods, wherein the fragment increases osteoclast fusion. The La protein can include a deletion of at most 5 amino acids, such as 1, 2, 3, 4, or 5 amino acids. In some embodiments, the La protein can include a deletion of up to 3 amino acids, such as 1, 2 or 3 amino acids of SEQ ID NO: 1, SEQ ID NO: 2, or SEQ ID NO: 7. In some embodiments, the deletion is within amino acids 376 to 408 of SEQ ID NO: 1. In one non-limiting example, the fragment is amino acids 6-242 of SEQ ID NO: 1.

La proteins of use can be prepared using recombinant methods, such as expression in host cells. Exemplary nucleic acid molecules can be prepared by cloning techniques (see below). Examples of appropriate cloning and sequencing techniques, and instructions sufficient to direct persons of skill through many cloning exercises are known (see, e.g., Sambrook et al. (Molecular Cloning: A Laboratory Manual, 4^{th} ed, Cold Spring Harbor, New York, 2012) and Ausubel et al. (In Current Protocols in Molecular Biology, John Wiley & Sons, New York, through supplement 104, 2013).

Hosts can include microbial, yeast, insect and mammalian organisms. Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as human). Exemplary cells of use include Escherichia coli, Bacillus subtilis, Saccharomyces cerevisiae, Salmonella typhimurium, SF9 cells, C129 cells, HEK 293 cells, Neurospora, and immortalized mammalian myeloid and lymphoid cell lines. Techniques for the propagation of mammalian cells in culture are well-known (see, e.g., Helgason and Miller (Eds.), 2012, Basic Cell Culture Protocols (Methods in Molecular Biology), 4^{th} Ed., Humana Press). Examples of commonly used mammalian host cell lines are VERO and HeLa cells, CHO cells, and W138, BHK, and COS cell lines, although cell lines may be used, such as cells designed to provide higher expression, desirable glycosylation patterns, or other features. In some embodiments, the host cells include HEK 293 cells or derivatives thereof, such as GnTI^{-/-} cells (ATCC® No. CRL-3022), or HEK-293F cells.

Transformation of a host cell with recombinant DNA can be carried out by conventional techniques. In some embodiments where the host is prokaryotic, such as, but not limited to, E. coli, competent cells which are capable of DNA uptake can be prepared from cells harvested after exponential growth phase and subsequently treated by the CaCl₂ method. Alternatively, heat shock, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or viral vectors can be used. Eukaryotic cells can also be co-transformed with polynucleotide sequences encoding a disclosed antigen, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Viral Expression Vectors, Springer press, Muzyczka ed., 2011). Appropriate expression systems such as plasmids and vectors of use in producing proteins in cells including higher eukaryotic cells such as the COS, CHO, HeLa and myeloma cell lines.

B. Nucleic Acid Molecules Encoding La and Vectors

Nucleic acid molecules encoding a La protein, variant or fragment are also of use in the disclosed methods. By introducing a nucleic acid encoding La protein, variant or fragment, the amount of La protein is increased, and thus the activity of La protein is also increased.

Nucleic acid molecules can be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill. RNA molecules are also of use.

The polynucleotides encoding the La protein, variant or fragment can include a recombinant DNA which is incorporated into a vector (such as an expression vector) into an autonomously replicating plasmid or virus or into the genomic DNA of a prokaryote or eukaryote, or which exists as a separate molecule (such as a cDNA) independent of other sequences. The nucleotides can be ribonucleotides, deoxyribonucleotides, or modified forms of either nucleotide. The term includes single and double forms of DNA. An exemplary nucleic acid molecule encoding SEQ ID NO: 1 is provided below:

| (SEQ ID NO: 3) | |
|---|---|
| 1 | atcttcttgg agcgctttag gctggccggc ggcgctggga ggtggagtcg ttgctgttgc |
| 61 | tgtttgtgag cctgtggcgc ggcttctgtg ggccggaacc ttaaagatag ccgcaatggc |
| 121 | tgaaaatggt gataatgaaa agatggctgc cctggaggcc aaaatctgtc atcaaattga |
| 181 | gtattatttt ggcgacttca atttgccacg ggacaagttt ctaaaggaac agataaaact |
| 241 | ggatgaaggc tgggtacctt tggagataat gataaaattc aacaggttga accgtctaac |
| 301 | aacagacttt aatgtaattg tggaagcatt gagcaaatcc aaggcagaac tcatggaaat |
| 361 | cagtgaagat aaaactaaaa tcagaaggtc tccaagcaaa cccctacctg aagtgactga |
| 421 | tgagtataaa aatgatgtaa aaaacagatc tgtttatatt aaaggcttcc caactgatgc |
| 481 | aactcttgat gacataaaag aatggttaga agataaaggt caagtactaa atattcagat |
| 541 | gagaagaaca ttgcataaag catttaaggg atcaattttt gttgtgtttg atagcattga |
| 601 | atctgctaag aaatttgtag agacccctgg ccagaagtac aaagaaacag acctgctaat |
| 661 | acttttcaag gacgattact ttgccaaaaa aaatgaagaa agaaaacaaa ataaagtgga |
| 721 | agctaaatta agagctaaac aggagcaaga agcaaaacaa aagttagaag aagatgctga |
| 781 | aatgaaatct ctagaagaaa agattggatg cttgctgaaa ttttcgggtg atttagatga |
| 841 | tcagacctgt agagaagatt tacacatact tttctcaaat catggtgaaa taaaatggat |
| 901 | agacttcgtc agaggagcaa aagaggggat aattctattt aaagaaaaag ccaaggaagc |
| 961 | attgggtaaa gccaaagatg caaataatgg taacctacaa ttaaggaaca aagaagtgac |
| 1021 | ttgggaagta ctagaaggag aggtggaaaa agaagcactg aagaaaataa tagaagacca |
| 1081 | acaagaatcc ctaaacaaat ggaagtcaaa aggtcgtaga tttaaaggaa aaggaaaggg |
| 1141 | taataaagct gcccagcctg ggtctggtaa aggaaaagta cagtttcagg gcaagaaaac |
| 1201 | gaaatttgct agtgatgatg aacatgatga acatgatgaa aatggtgcaa ctggacctgt |
| 1261 | gaaaagagca agagaagaaa cagacaaaga agaacctgca tccaaacaac agaaaacaga |
| 1321 | aaatggtgct ggagaccagt agtttagtaa accaattttt tattcatttt aaataggttt |
| 1381 | taaacgactt ttgtttgcgg ggcttttaaa aggaaaaccg aattaggtcc acttcaatgt |
| 1441 | ccacctgtga gaaaggaaaa atttttttgt tgtttaactt gtctttttgt tatgcaaatg |
| 1501 | agatttcttt gaatgtattg ttctgtttgt gttatttcag atgattcaaa tatcaaaagg |
| 1561 | aagattcttc cattaaattg cctttgtaat atgagaatgt attagtacaa actaactaat |
| 1621 | aaaatatata ctatatgaaa agagcaaaaa |

An exemplary nucleic acid sequence encoding Peptide 2 (see below) is shown below:

(SEQ ID NO: 10)
gctaaatta agagctaaac aggagcaaga agcaaaacaa
aagttagaag aagatgctga aatgaaatct ctagaagaaa
agattggatg c

An exemplary nucleic acid sequence encoding Peptide 2 (see below) is shown below:

(SEQ ID NO: 11)
ggag aggtggaaaa agaagcactg aagaaaataa
tagaagacca acaagaatcc ctaaacaaat ggaagtcaaa
aggtcgtaga tttaaa

Polynucleotides encoding a La protein, variant or fragments thereof are of use in the disclosed methods include DNA, cDNA, and RNA sequences that encode the La protein. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (e.g., L. Stryer, 1988, Biochemistry, 3.sup.rd Edition, W.H. 5 Freeman and Co., NY). Degenerate variants are also of use in the methods disclosed herein.

Additional nucleic acid molecules encoding a La protein, variant or fragment can readily be produced by one of skill in the art using the amino acid sequences provided herein and the genetic code. Nucleic acid sequences encoding the La protein, variant or fragment can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al., Nucl. Acids Res. 12:6159-6168, 1984 and the solid support method of U.S. Pat. No. 4,458,066. Chemical synthesis produces a single-strand (ss) oligonucleotide, which can be converted into double-strand (ds) DNA by hybridization with a complementary sequence or by polymerization with a DNA polymerase using the single strand as a template. Exemplary nucleic acids that include sequences encoding a La protein, variant or fragment can be prepared by cloning techniques.

A nucleic acid molecule encoding a La protein, variant or fragment can be cloned or amplified by in vitro methods, such as the polymerase chain reaction (PCR), the ligase chain reaction (LCR), the transcription-based amplification system (TAS), the self-sustained sequence replication system (3SR), and the Qβ replicase amplification system (QB). For example, a polynucleotide encoding the La protein, variant or fragment can be isolated by a polymerase chain reaction of cDNA using primers based on the DNA sequence of the molecule. A wide variety of cloning and in vitro amplification methodologies can be used. PCR methods are described in, for example, U.S. Pat. No. 4,683,195; Mullis et al., Cold Spring Harbor Symp. Quant. Biol. 51:263, 1987; and Erlich, ed., PCR Technology, (Stockton Press, N Y, 1989). Polynucleotides also can be isolated by screening genomic or cDNA libraries with probes selected from the sequences of the desired polynucleotide under stringent hybridization conditions.

Typically, a polynucleotide sequence encoding a La protein, variant or fragment is operably linked to transcriptional control sequences including, for example a promoter and a polyadenylation signal. Any promoter can be used that is a polynucleotide sequence recognized by the transcriptional machinery of the host cell (or introduced synthetic machinery) that is involved in the initiation of transcription. A polyadenylation signal is a polynucleotide sequence that directs the addition of a series of nucleotides on the end of the mRNA transcript for proper processing and trafficking of the transcript out of the nucleus into the cytoplasm for translation.

Exemplary promoters include viral promoters, such as cytomegalovirus immediate early gene promoter (“CMV”), herpes simplex virus thymidine kinase (“tk”), SV40 early transcription unit, polyoma, retroviruses, papilloma virus, hepatitis B virus, and human and simian immunodeficiency viruses. Other promoters include promoters isolated from mammalian genes, such as the immunoglobulin heavy chain, immunoglobulin light chain, T cell receptor, HLA DQ α and DQ β, β-interferon, interleukin-2, interleukin-2 receptor, MHC class II, HLA-DRα, β-actin, muscle creatine kinase, prealbumin (transthyretin), elastase I, metallothionein, collagenase, albumin, fetoprotein, β-globin, c-fos, c-HA-ras, neural cell adhesion molecule (NCAM), al-antitrypsin, H2B (TH2B) histone, type I collagen, glucose-regulated proteins (GRP94 and GRP78), rat growth hormone, human serum amyloid A (SAA), troponin I (TNI), platelet-derived growth factor, and dystrophin, as well as promoters specific for bone cells.

The promoter can be either inducible or constitutive. An inducible promoter is a promoter that is inactive or exhibits low activity except in the presence of an inducer substance. Additional examples of promoters include, but are not limited to, MT II, MMTV, collagenase, stromelysin, SV40, murine MX gene, α-2-macroglobulin, MHC class I gene h-2 kb, HSP70, proliferin, tetracycline inducible, tumor necrosis factor, or thyroid stimulating hormone gene promoter. One example of an inducible promoter is the interferon inducible ISG54 promoter (see Bluyssen et al., Proc. Natl Acad. Sci. 92: 5645-5649, 1995, herein incorporated by reference). In some embodiments, the promoter is a constitutive promoter that results in high levels of transcription upon introduction into a host cell in the absence of additional factors. In some embodiments, the promoter is a bone specific promoter, such as an osteocalcin promoter, osteopontin promoter, or osteonectin promoter (also see Grienberg and Benayahu, BMC Genomics, 6, 46 (2005), doi.org/10.1186/1471-2164-6-46 005, herein incorporated by reference in its entirety). Moreover, in some embodiments, the promoter is osteoclast or osteoclast progenitor specific promoter, such as a cathepsin K promotor, a tartrate-resistant acid phosphatase promotor, a lysozyme M promotor, a receptor activator of nuclear factor kappa B promotor, a macrophage surface antigen-1 promotor, or macrophage colony-stimulating factor receptor promotor, see the internet, .ncbi.nlm.nih.gov/pmc/articles/PMC6397767/.

Optionally, transcription control sequences include one or more enhancer elements, which are binding recognition sites for one or more transcription factors that increase transcription above that observed for the minimal promoter alone, and also be operably linked to the polynucleotide encoding the nucleic acid molecule encoding the La protein. With regard to the nucleic acid molecule encoding the La protein, introns can also be included that help stabilize mRNA and increase expression.

It may be desirable to include a polyadenylation signal to effect proper termination and polyadenylation of the La gene transcript. Exemplary polyadenylation signals have been isolated from beta globin, bovine growth hormone, SV40, and the herpes simplex virus thymidine kinase genes.

A nucleic acid molecule encoding a La protein, variant or fragment can be included in a viral vector, for example for expression of the protomer to produce the corresponding La protein, variant or fragment in a host cell, or for administration to a subject as disclosed herein. Typically, such viral vectors include a nucleic acid molecule encoding a La protein, variant or fragment. In some examples, the viral vector encoding the La protein, variant or fragment can be replication-competent. For example, the viral vector can have a mutation (e.g., insertion of nucleic acid encoding the protomer) in the viral genome that attenuates, but does not completely block viral replication in host cells.

Various viral vectors which can be utilized for nucleic acid based therapy as taught herein include adenovirus or adeno-associated virus, herpes virus, vaccinia, or an RNA virus such as a retrovirus (including HVJ, see Kotani et al., Curr. Gene Ther. 4:183-194, 2004). In one embodiment, the retroviral vector is a derivative of a murine or avian retrovirus, or a human or primate lentivirus. Examples of retroviral vectors in which a foreign gene can be inserted include, but are not limited to: Moloney murine leukemia virus (MoMLV), Harvey murine sarcoma virus (HaMuSV), murine mammary tumor virus (MuMTV), and Rous Sarcoma Virus (RSV). In one embodiment, when the subject is a human, a vector such as the gibbon ape leukemia virus (GaLV) can be utilized. A pseudotyped retroviral vector can be utilized that includes a heterologous envelope gene.

A number of additional retroviral vectors can incorporate multiple genes. All of these vectors can transfer or incorporate a gene for a selectable marker so that transduced cells can be identified and generated. By inserting a nucleic acid encoding La protein, variant or fragment into the viral vector, along with another gene which can serve as viral envelope protein and also can encode the ligand for a receptor on a specific target cell, for example, the vector is now target specific. Retroviral vectors can be made target specific by modifications of the envelope protein by attaching, for example, a sugar, a glycolipid, or a protein. In one specific, non-limiting example, targeting is accomplished by using an antibody to target the retroviral vector.

Since recombinant retroviruses are non-replicating by design, they require assistance in order to produce infectious vector particles. This assistance can be provided, for example, by using helper cell lines that contain plasmids encoding all of the structural genes of the retrovirus under the control of regulatory sequences within the long terminal repeat (LTR). These plasmids are missing a nucleotide sequence which enables the packaging mechanism to recognize an RNA transcript for encapsidation. Helper cell lines which have deletions of the packaging signal include, but are not limited to ψ2, PA317, and PA12, for example. These cell lines produce empty virions, since no genome is packaged. If a retroviral vector is introduced into such cells in which the packaging signal is intact, but the structural genes are replaced by other genes of interest, the vector can be packaged and vector virion produced.

Alternatively, NIH 3T3 or other tissue culture cells can be directly transfected with plasmids encoding the retroviral structural genes gag, pol and env, by conventional transfection methods. These cells are then transfected with the vector plasmid containing the genes of interest. The resulting cells release the retroviral vector into the culture medium.

Adenovirus vectors are also of use. The adenovirus vectors include replication competent, replication deficient, gutless forms thereof. Defective viruses, such as adenovirus vectors or adeno-associated virus (AAV) vectors, that entirely or almost entirely lack viral genes, can be used. Use of defective viral vectors allows for administration to specific cells without concern that the vector can infect other cells. The AAV vectors of use are replication deficient. Without being bound by theory, adenovirus vectors are known to exhibit strong expression in vitro, excellent titer, and the ability to transduce dividing and non-dividing cells in vivo (Hitt et al., Adv in Virus Res 55:479-505, 2000). When used in vivo these vectors lead to strong but transient gene expression due to immune responses elicited to the vector backbone. In some non-limiting examples, a vector of use is an attenuated adenovirus vector, such as the vector described by Stratford-Perricaudet et al. (J. Clin. Invest., 90:626-630 1992; La Salle et al., Science 259:988-990, 1993); or a defective AAV vector (Samulski et al., J. Virol., 61:3096-3101, 1987; Samulski et al., J. Virol., 63:3822-3828, 1989; Lebkowski et al., Mol. Cell. Biol., 8:3988-3996, 1988).

Recombinant AAV vectors are characterized in that they are capable of directing the expression and the production of the selected transgenic products in targeted cells. Thus, the recombinant vectors comprise at least all of the sequences of AAV essential for encapsidation and the physical structures for infection of target cells.

AAV belongs to the family Parvoviridae and the genus Dependovirus. AAV is a small, non-enveloped virus that packages a linear, single-stranded DNA genome. Both sense and antisense strands of AAV DNA are packaged into AAV capsids with equal frequency. In some embodiments, the AAV DNA includes a nucleic acid including a promoter operably linked to a nucleic acid molecule encoding a La protein, variant or fragment. Further provided are recombinant vectors, such as recombinant adenovirus vectors and recombinant adeno-associated virus (rAAV) vectors comprising a nucleic acid molecule(s) disclosed herein. In some embodiments, the AAV is rAAV8, and/or AAV2. However, the AAV serotype can be any other suitable AAV serotype, such as AAV1, AAV2, AAV3, AAV4, AAV5, AAV6, AAV7, AAV9, AAV10, AAV11 or AAV12, or a hybrid of two or more AAV serotypes.

Additional viral vectors are also available for expression of La, including polyoma, i.e., SV40 (Madzak et al., 1992, J. Gen. Virol., 73:15331536), herpes viruses including HSV and EBV and CMV (Margolskee, 1992, Curr. Top. Microbiol. Immunol., 158:67-90; Johnson et al., 1992, J. Virol., 66:29522965; Fink et al., 1992, Hum. Gene Ther. 3:11-19; Breakfield et al., 1987, Mol. Neurobiol., 1:337-371; Fresse et al., 1990, Biochem. Pharmacol., 40:2189-2199), Sindbis viruses (H. Herweijer et al., 1995, Human Gene Therapy 6:1161-1167; U.S. Pat. Nos. 5,091,309 and 5,2217,879), alphaviruses (S. Schlesinger, 1993, Trends Biotechnol. 11:18-22; I. Frolov et al., 1996, Proc. Natl. Acad. Sci. USA 93:11371-11377) and retroviruses of avian (Brandyopadhyay et al., 1984, Mol. Cell Biol., 4:749-754; Petropouplos et al., 1992, J. Virol., 66:3391-3397), murine (Miller, 1992, Curr. Top. Microbiol. Immunol., 158:1-24; Miller et al., 1985, Mol. Cell Biol., 5:431-437; Sorge et al., 1984, Mol. Cell Biol., 4:1730-1737; Mann et al., 1985, J. Virol., 54:401-407), and human origin (Page et al., 1990, J. Virol., 64:5370-5276; Buchschalcher et al., 1992, J. Virol., 66:2731-2739). Baculovirus (Autographa californica multinuclear polyhedrosis virus; AcMNPV) vectors are also known in the art, and may be obtained from commercial sources (such as PharMingen, San Diego, Calif.; Protein Sciences Corp., Meriden, Conn.; Stratagene, La Jolla, Calif.).

Another targeted delivery system for a polynucleotide encoding La protein, variant or fragment is a colloidal dispersion system. These systems are also of use in the disclosed methods.

Colloidal dispersion systems include macromolecule complexes, nanocapsules, microspheres, beads, and lipid-based systems including oil-in-water emulsions, micelles, mixed micelles, and liposomes. One colloidal dispersion system is a liposome. Liposomes are artificial membrane vesicles which are useful as delivery vehicles in vitro and in vivo. It has been shown that large unilamellar vesicles (LUV), which range in size from about 0.2-4 microns, can encapsulate a substantial percentage of an aqueous buffer containing large macromolecules. RNA, DNA and intact virions can be encapsulated within the aqueous interior and be delivered to cells in a biologically active form (Fraley et al., Trends Biochem. Sci. 6:77, 1981). In addition to mammalian cells, liposomes have been used for delivery of polynucleotides in plant, yeast and bacterial cells. In order for a liposome to be an efficient gene transfer vehicle, the following characteristics should be present: (1) encapsulation of the nucleic acid of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino et al., Biotechniques 6:682, 1988).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidyl-glycerols, where the lipid moiety contains from 14-18 carbon atoms, particularly from 16-18 carbon atoms, and is saturated. Illustrative phospholipids include, for example, phosphatidylcholine, dipalmitoylphosphatidylcholine and distearoylphosphatidylcholine.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticuloendothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

Another targeting delivery system is the use of biodegradable and biocompatible polymer scaffolds (see Jang et al., Expert Rev. Medical Devices 1:127-138, 2004) for use in the bone. These scaffolds usually contain a mixtures of one or more biodegradable polymers, for example and without limitation, saturated aliphatic polyesters, such as poly(lactic acid) (PLA), poly(glycolic acid), or poly(lactic-co-glycolide) (PLGA) copolymers, unsaturated linear polyesters, such as polypropylene fumarate (PPF), or microorganism produced aliphatic polyesters, such as polyhydroxyalkanoates (PHA), (see Rezwan et al., Biomaterials 27:3413-3431, 2006; Laurencin et al., Clin. Orthopaed. Rel. Res. 447:221-236). By varying the proportion of the various components, polymeric scaffolds of different mechanical properties are obtained. A commonly used scaffold contains a ratio of PLA to PGA is 75:25, but this ratio may change depending upon the specific application. Other commonly used scaffolds include surface bioeroding polymers, such as poly(anhydrides), such as trimellitylimidoglycine (TMA-gly) or pyromellitylimidoalanine (PMA-ala), or poly(phosphazenes), such as high molecular weight poly(organophasphazenes) (P[PHOS]), and bioactive ceramics. The gradual biodegradation of these scaffolds allows the gradual release of drugs or gene from the scaffold. Thus, an advantage of these polymeric carriers is that they represent not only a scaffold but also a drug or gene delivery system. This system is applicable to the delivery of plasmid DNA and also applicable to viral vectors, such as AAV or retroviral vectors, as well as transposon-based vectors.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand.

C. Chemical Compounds and Small Molecule Agonists

La protein agonists include molecules that are identified from large libraries of both natural product or synthetic (or semi-synthetic) extracts or chemical libraries. Screening methods that detect increases in La activity, such as by measuring osteoclast fusion, are useful for identifying compounds from a variety of sources for activity. The initial screens may be performed using a diverse library of compounds, a variety of other compounds and compound libraries. Thus, molecules that increase the activity of La protein can be identified. These small molecules can be identified from combinatorial libraries, natural product libraries, or other small molecule libraries. In addition, La agonists can be identified as compounds from commercial sources, as well as commercially available analogs of identified inhibitors.

The precise source of test extracts or compounds is not critical to the identification of La protein agonists. Accordingly, La protein agonists can be identified from virtually any number of chemical extracts or compounds. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). La protein agonists can be identified from synthetic compound libraries that are commercially available from a number of companies including Maybridge Chemical Co. (Trevillet, Cornwall, UK), Comgenex (Princeton, N. J.), Brandon Associates (Merrimack, N.H.), and Microsource (New Milford, Conn.). La protein agonists can be identified from a rare chemical library, such as the library that is available from Aldrich (Milwaukee, Wis.). La protein agonists can be identified in libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). Natural and synthetically produced libraries and compounds are readily modified through conventional chemical, physical, and biochemical means.

Useful compounds may be found within numerous chemical classes, though typically they are organic compounds, including small organic compounds. Small organic compounds have a molecular weight of more than 50 yet less than about 2,500 daltons, such as less than about 750 or less than about 350 daltons can be utilized in the methods disclosed herein. Exemplary classes include heterocycles, peptides, saccharides, steroids, and the like. The compounds may be modified to enhance efficacy, stability, pharmaceutical compatibility, and the like.

III. Agents that Decrease La Protein Function and/or Activity Methods are disclosed herein that decrease osteoclast fusion in the subject. The method can reduce bone resorption. These methods use an agent that decreases La protein expression or activity in the subject.

Exemplary Agents are Disclosed Below.

A. Antibodies and Antigen Binding Fragments Thereof An agent that decreases La protein activity can be an antagonistic antibody that specifically binds the La protein, such as, but not limited to, a monoclonal antibody. Antibodies that specifically bind La protein are commercially available. In some embodiments, the antibody binds La protein and decreases osteoclast fusion. Exemplary antibodies are disclosed Example 8.

Antibodies that specifically bind and substantially reduce or inhibit La protein activity (such as a reduction of at least 20%, at least 40%, at least 50%, at least 75%, at least 80%, at least 90%, at least 95%, at least 98% or even 100%) are of use in the methods disclosed herein. Antibodies include monoclonal antibodies, human antibodies, humanized antibodies, deimmunized antibodies, and immunoglobulin (Ig) fusion proteins. Fully human and humanized antibodies that bind La protein can also be produced using methods known to those of skill in the art.

Polyclonal antagonistic antibodies can be prepared, such as by immunizing a suitable subject (such as a human subject or a veterinary subject) with a La protein. The anti-La protein antibody titer in the immunized subject can be monitored over time, such as with an enzyme linked immunosorbent assay (ELISA) using immobilized La protein or an epitope thereof. In one example, the antibody molecules that specifically bind La protein can be isolated from a mammal (such as from serum) and further purified, for example using protein A chromatography to isolate IgG antibodies. In some embodiments, the antibody can also be selected using a functional assay, such as to detect inhibition of osteoclast fusion.

Antibody-producing cells can be obtained from a subject, such as an immunized subject, and used to prepare monoclonal antibodies (see Kohler and Milstein Nature 256:495 49, 1995; Brown et al., J. Immunol. 127:539 46, 1981; Cole et al., Monoclonal Antibodies and Cancer Therapy, Alan R. Liss, Inc., pp. 77 96, 1985; Gefter, M. L. et al. (1977) Somatic Cell Genet. 3:231 36; Kenneth, R. H. in Monoclonal Antibodies: A New Dimension In Biological Analyses. Plenum Publishing Corp., New York, N.Y. (1980); Kozbor et al. Immunol. Today 4:72, 1983; Lerner, E. A. (1981) Yale J. Biol. Med. 54:387 402; Yeh et al., Proc. Natl. Acad. Sci. 76:2927 31, 1976). In one example, an immortal cell line (typically a myeloma) is fused to lymphocytes (typically splenocytes) from a mammal immunized with La protein, and the culture supernatants of the resulting hybridoma cells are screened to identify a hybridoma producing a monoclonal antibody that specifically binds to the polypeptide of interest and inhibits a function of the polypeptide.

In one embodiment, to produce a hybridoma, an immortal cell line (such as a myeloma cell line) is derived from the same mammalian species as the lymphocytes. For example, murine hybridomas can be made by fusing lymphocytes from a mouse immunized with a La protein, or an epitope thereof, with an immortalized mouse cell line. In one example, a mouse myeloma cell line is utilized that is sensitive to culture medium containing hypoxanthine, aminopterin and thymidine (“HAT medium”). Any of a number of myeloma cell lines can be used as a fusion partner, including, for example, P3-NS1/1-Ag4-1, P3-x63-Ag8.653 or Sp2/O-Ag14 myeloma lines, which are available from the American Type Culture Collection (ATCC), Rockville, MD. HAT-sensitive mouse myeloma cells can be fused to mouse splenocytes using polyethylene glycol (“PEG”). Hybridoma cells resulting from the fusion are then selected using HAT medium, which kills unfused (and unproductively fused) myeloma cells. Hybridoma cells producing a monoclonal antibody of interest can be detected, for example, by screening the hybridoma culture supernatants for the production antibodies that bind a La polypeptide, such as by using an immunological assay (such as an enzyme-linked immunosorbant assay (ELISA) or radioimmunoassay (RIA).

As an alternative to preparing monoclonal antibody-secreting hybridomas, a monoclonal antibody that specifically binds La protein can be identified and isolated by screening a recombinant combinatorial immunoglobulin library (such as an antibody phage display library) with La protein, or an epitope thereof, to isolate immunoglobulin library members that specifically bind the polypeptide. Library members can be selected that have particular activities, such as binding La protein, or inhibiting osteoclast fusion in an in vitro assay. Kits for generating and screening phage display libraries are commercially available (such as, but not limited to, Pharmacia and Stratagene). Examples of methods and reagents particularly amenable for use in generating and screening antibody display library can be found in, for example, U.S. Pat. No. 5,223,409; PCT Publication No. WO 90/02809; PCT Publication No. WO 91/17271; PCT Publication No. WO 92/18619; PCT Publication WO 92/20791; PCT Publication No. WO 92/15679; PCT Publication No. WO 92/01047; PCT Publication WO 93/01288; PCT Publication No. WO 92/09690; Barbas et al., Proc. Natl. Acad. Sci. USA 88:7978 7982, 1991; Hoogenboom et al., Nucleic Acids Res. 19:4133 4137, 1991. Suitable assays for monitoring osteoclast fusion are disclosed, for example, in the Examples section.

In one example the sequence of the specificity determining regions of each CDR is determined. Residues outside the SDR (specificity determining region, e.g., the non-ligand contacting sites) are substituted. For example, in any of the CDR sequences, at most one, two or three amino acids can be substituted. The production of chimeric antibodies, which include a framework region from one antibody and the CDRs from a different antibody, is known in the art. For example, humanized antibodies can be produced. The antibody or antibody fragment can be a humanized immunoglobulin having CDRs from a donor monoclonal antibody that binds La protein, or an epitope thereof, and immunoglobulin and heavy and light chain variable region frameworks from human acceptor immunoglobulin heavy and light chain frameworks.

Humanized monoclonal antibodies can be produced by transferring CDRs from heavy and light variable chains of the donor mouse immunoglobulin (that specifically binds La protein) into a human variable domain, and then substituting human residues in the framework regions when required to retain affinity. The use of antibody components derived from humanized monoclonal antibodies obviates potential problems associated with the immunogenicity of the constant regions of the donor antibody. Techniques for producing humanized monoclonal antibodies are described, for example, by Jones et al., Nature 321:522, 1986; Riechmann et al., Nature 332:323, 1988; Verhoeyen et al., Science 239:1534, 1988; Carter et al., Proc. Natl. Acad. Sci. U.S.A. 89:4285, 1992; Sandhu, Crit. Rev. Biotech. 12:437, 1992; and Singer et al., J. Immunol. 150:2844, 1993. The antibody may be of any isotype, but in several embodiments the antibody is an IgG, including but not limited to, IgG₁, IgG₂, IgG₃ and IgG₄.

In one embodiment, the sequence of the humanized immunoglobulin heavy chain variable region framework can be at least about 65% identical to the sequence of the donor immunoglobulin heavy chain variable region framework. Thus, the sequence of the humanized immunoglobulin heavy chain variable region framework can be at least about 75%, at least about 85%, at least about 99% or at least about 95%, identical to the sequence of the donor immunoglobulin heavy chain variable region framework. Human framework regions, and mutations that can be made in humanized antibody framework regions, are known in the art (see, for example, in U.S. Pat. No. 5,585,089, incorporated herein by reference in its entirety).

Exemplary human antibodies are LEN and 21/28 CL. The sequences of the many human heavy and light chain frameworks are known. Generally, an antibody, such as a human or humanized antibody specifically binds to La protein, and/or an epitope thereof, with an affinity constant of at least 10⁷ M⁻¹, such as at least 10⁸ M⁻¹ at least 5×10⁸ M⁻¹ or at least 10⁹ M⁻¹. In several examples, the antibody specifically binds La protein, or an epitope thereof, with an affinity constant of at least 10⁸ M⁻¹ at least 5×10⁸ M⁻¹ or at least 10⁹ M⁻¹. These antibodies can inhibit osteoclast fusion, as compared to a control, such as osteoclast fusion in the absence of the antibody or with a control isotype matched antibody. The antibody can be a fully human antibody.

Antibodies, such as murine monoclonal antibodies, chimeric antibodies, and humanized antibodies, include full length molecules as well as fragments thereof, such as Fab, F(ab′)₂, and Fv which include a heavy chain and light chain variable region and are capable of binding specific epitope determinants. These antibody fragments retain some ability to selectively bind with their antigen or receptor. These fragments include:
(1) Fab, the fragment which contains a monovalent antigen-binding fragment of an antibody molecule, can be produced by digestion of whole antibody with the enzyme papain to yield an intact light chain and a portion of one heavy chain;
(2) Fab′, the fragment of an antibody molecule can be obtained by treating whole antibody with pepsin, followed by reduction, to yield an intact light chain and a portion of the heavy chain; two Fab′ fragments are obtained per antibody molecule;
(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;
(4) Fv, a genetically engineered fragment containing the variable region of the light chain and the variable region of the heavy chain expressed as two chains; and
(5) Single chain antibody (such as scFv), defined as a genetically engineered molecule containing the variable region of the light chain, the variable region of the heavy chain, linked by a suitable polypeptide linker as a genetically fused single chain molecule.

Methods of making these fragments are known (see for example, Harlow and Lane, Antibodies: A Laboratory Manual, Cold Spring Harbor Laboratory, New York, 1988). In several examples, the variable region includes the variable region of the light chain and the variable region of the heavy chain expressed as individual polypeptides. Fv antibodies are typically about 25 kDa and contain a complete antigen-binding site with three CDRs per each heavy chain and each light chain. To produce these antibodies, the V_{H} and the V_{L} can be expressed from two individual nucleic acid constructs in a host cell. If the V_{H} and the V_{L} are expressed non-contiguously, the chains of the Fv antibody are typically held together by noncovalent interactions. However, these chains tend to dissociate upon dilution, so methods have been developed to crosslink the chains through glutaraldehyde, intermolecular disulfides, or a peptide linker. Thus, in one example, the Fv can be a disulfide stabilized Fv (dsFv), wherein the heavy chain variable region and the light chain variable region are chemically linked by disulfide bonds.

In an additional example, the Fv fragments comprise V_{H} and V_{L} chains connected by a peptide linker. These single-chain antigen binding proteins (scFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing scFvs are known (see Whitlow et al., Methods: a Companion to Methods in Enzymology, Vol. 2, page 97, 1991; Bird et al., Science 242:423, 1988; U.S. Pat. No. 4,946,778; Pack et al., Bio/Technology 11:1271, 1993; and Sandhu, supra).

Antibody fragments can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. Nos. 4,036,945 and 4,331,647, and references contained therein; Nisonhoff et al., Arch. Biochem. Biophys. 89:230, 1960; Porter, Biochem. J. 73:119, 1959; Edelman et al., Methods in Enzymology, Vol. 1, page 422, Academic Press, 1967; and Coligan et al. at sections 2.8.1-2.8.10 and 2.10.1-2.10.4).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. Any of the antigen binding fragments described herein are of use.

Conservative variants of the antibodies can be produced. Such conservative variants employed in antibody fragments, such as dsFv fragments or in scFv fragments, will retain critical amino acid residues necessary for correct folding and stabilizing between the V_{H} and the V_{L} regions, and will retain the charge characteristics of the residues in order to preserve the low pI and low toxicity of the molecules. Amino acid substitutions (such as at most one, at most two, at most three, at most four, or at most five amino acid substitutions) can be made in the V_{H} and the V_{L} regions to increase yield. In some embodiments, these substitutions are made in the framework regions, and are not made in the CDRs. A table of conservative amino acid substitutions is provided above. One of skill in the art can readily review the amino acid sequence of an antibody of interest, locate one or more of the amino acids in the brief table above, identify a conservative substitution, and produce the conservative variant using molecular techniques.

Effector molecules, such as therapeutic, diagnostic, or detection moieties can be linked to an antagonistic antibody that specifically binds La protein, using any number of means. Both covalent and noncovalent attachment means may be used. The procedure for attaching an effector molecule to an antibody varies according to the chemical structure of the effector. Polypeptides typically contain a variety of functional groups; such as carboxylic acid (COOH), free amine (—NH₂) or sulfhydryl (—SH) groups, which are available for reaction with a suitable functional group on an antibody to result in the binding of the effector molecule. Alternatively, the antibody is derivatized to expose or attach additional reactive functional groups. The derivatization may involve attachment of any of a number of linker molecules such as those available from Pierce Chemical Company, Rockford, IL. The linker can be any molecule used to join the antibody to the effector molecule. The linker is capable of forming covalent bonds to both the antibody and to the effector molecule. Suitable linkers include, but are not limited to, straight or branched-chain carbon linkers, heterocyclic carbon linkers, or peptide linkers. Where the antibody and the effector molecule are polypeptides, the linkers may be joined to the constituent amino acids through their side groups (such as through a disulfide linkage to cysteine) or to the alpha carbon amino and carboxyl groups of the terminal amino acids.

Nucleic acid sequences encoding the antibodies can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al., Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. Longer sequences may be obtained by the ligation of shorter sequences generated by chemical synthesis.

Exemplary nucleic acids encoding sequences encoding an antagonistic antibody that specifically binds La protein can be prepared by cloning techniques. Examples of appropriate cloning and sequencing techniques, and instructions sufficient to direct persons of skill through many cloning exercises are found in Sambrook et al., supra, Berger and Kimmel (eds.), supra, and Ausubel, supra. Product information from manufacturers of biological reagents and experimental equipment also provide useful information. Such manufacturers include the SIGMA Chemical Company (Saint Louis, MO), R&D Systems (Minneapolis, MN), Pharmacia Amersham (Piscataway, NJ), CLONTECH Laboratories, Inc. (Palo Alto, CA), Chem Genes Corp., Aldrich Chemical Company (Milwaukee, WI), Glen Research, Inc., GIBCO BRL Life Technologies, Inc. (Gaithersburg, MD), Fluka Chemica-Biochemika Analytika (Fluka Chemie AG, Buchs, Switzerland), Invitrogen (San Diego, CA), and Applied Biosystems (Foster City, CA), as well as other commercial sources.

Nucleic acids can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are known.

In one example, an antibody of use is prepared by inserting the cDNA, which encodes a variable region from an antagonistic antibody that specifically binds La protein, into a vector which comprises the cDNA encoding an effector molecule (EM). The insertion is made so that the variable region and the EM are read in frame so that one continuous polypeptide is produced. Thus, the encoded polypeptide contains a functional Fv region and a functional EM region. In one embodiment, cDNA encoding a detectable marker (such as an enzyme) is ligated to a scFv so that the marker is located at the carboxyl terminus of the scFv. In another example, a detectable marker is located at the amino terminus of the scFv. In a further example, cDNA encoding a detectable marker is ligated to a heavy chain variable region of an antagonistic antibody that specifically binds La protein, so that the marker is located at the carboxyl terminus of the heavy chain variable region. The heavy chain-variable region can subsequently be ligated to a light chain variable region of the antibody that specifically binds La protein using disulfide bonds. In a yet another example, cDNA encoding a marker is ligated to a light chain variable region of an antagonistic antibody that binds La protein, so that the marker is located at the carboxyl terminus of the light chain variable region. The light chain-variable region can subsequently be ligated to a heavy chain variable region of the antagonistic antibody that specifically binds La protein using disulfide bonds.

Once the nucleic acids encoding the La antagonistic antibody or functional fragment thereof are isolated and cloned, the protein can be expressed in a recombinantly engineered cell such as bacteria, plant, yeast, insect and mammalian cells. One or more DNA sequences encoding the antibody or functional fragment thereof can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known.

Polynucleotide sequences encoding the antibody or functional fragment thereof (such as an scFV) can be operatively linked to expression control sequences. An expression control sequence operatively linked to a coding sequence is ligated such that expression of the coding sequence is achieved under conditions compatible with the expression control sequences. The expression control sequences include, but are not limited to appropriate promoters, enhancers, transcription terminators, a start codon (i.e., ATG) in front of a protein-encoding gene, splicing signal for introns, maintenance of the correct reading frame of that gene to permit proper translation of mRNA, and stop codons. RNA encoding the disclosed antibodies are also of use.

The polynucleotide sequences encoding the antibody or functional fragment thereof can be inserted into an expression vector including, but not limited to a plasmid, virus or other vehicle that can be manipulated to allow insertion or incorporation of sequences and can be expressed in either prokaryotes or eukaryotes. Hosts can include microbial, yeast, insect and mammalian organisms. Methods of expressing DNA sequences having eukaryotic or viral sequences in prokaryotes are well known in the art. Biologically functional viral and plasmid DNA vectors capable of expression and replication in a host are known.

Transformation of a host cell with recombinant DNA may be carried out by conventional techniques. Where the host is prokaryotic, such as E. coli, competent cells which are capable of DNA uptake can be prepared from cells harvested after exponential growth phase and subsequently treated by the CaCl₂ method. Alternatively, MgCl₂ can be used. Transformation can also be performed after forming a protoplast of the host cell if desired, or by electroporation.

When the host is a eukaryote, such methods of transfection of DNA as calcium phosphate coprecipitates, mechanical procedures such as microinjection, electroporation, insertion of a plasmid encased in liposomes, or virus vectors, may be used. Eukaryotic cells can also be co-transformed with polynucleotide sequences encoding the antibody of functional fragment thereof and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982). One of skill in the art can readily use expression systems such as plasmids and vectors of use in producing proteins in cells including higher eukaryotic cells such as the COS, CHO, HeLa and myeloma cell lines.

Isolation and purification of a recombinantly expressed polypeptide can be carried out by conventional means including preparative chromatography and immunological separations. Once expressed, the recombinant antibodies can be purified according to standard procedures of the art, including ammonium sulfate precipitation, affinity columns, column chromatography, and the like (see, generally, R. Scopes, Protein Purification, Springer-Verlag, N.Y., 1982). Substantially pure compositions of at least about 90 to 95% homogeneity are disclosed herein, and 98 to 99% or more homogeneity can be used for pharmaceutical purposes. Once purified, partially or to homogeneity as desired, if to be used therapeutically, the polypeptides should be substantially free of endotoxin.

Methods for expression of single chain antibodies and/or refolding to an appropriate active form, including single chain antibodies, from bacteria such as E. coli have been described and are applicable to the antibodies disclosed herein. See, Buchner et al., Anal. Biochem. 205:263-270, 1992; Pluckthun, Biotechnology 9:545, 1991; Huse et al., Science 246:1275, 1989 and Ward et al., Nature 341:544, 1989, all incorporated by reference herein.

Often, functional heterologous proteins from E. coli or other bacteria are isolated from inclusion bodies and require solubilization using strong denaturants, and subsequent refolding. During the solubilization step, a reducing agent must be present to separate disulfide bonds. An exemplary buffer with a reducing agent is: 0.1 M Tris pH 8, 6 M guanidine, 2 mM EDTA, 0.3 M DTE (dithioerythritol). Reoxidation of the disulfide bonds can occur in the presence of low molecular weight thiol reagents in reduced and oxidized form, as described in Saxena et al., Biochemistry 9: 5015-5021, 1970, incorporated by reference herein, and especially as described by Buchner et al., supra.

Renaturation is typically accomplished by dilution (for example, 100-fold) of the denatured and reduced protein into refolding buffer. An exemplary buffer is 0.1 M Tris, pH 8.0, 0.5 M L-arginine, 8 mM oxidized glutathione (GSSG), and 2 mM EDTA.

As a modification to the two chain antibody purification protocol, the heavy and light chain regions are separately solubilized and reduced and then combined in the refolding solution. An exemplary yield is obtained when these two proteins are mixed in a molar ratio such that a 5 fold molar excess of one protein over the other is not exceeded. It is desirable to add excess oxidized glutathione or other oxidizing low molecular weight compounds to the refolding solution after the redox-shuffling is completed.

In addition to recombinant methods, the antagonistic antibodies and functional fragments thereof that are disclosed herein can also be constructed in whole or in part using standard peptide synthesis. Solid phase synthesis of the polypeptides of less than about 50 amino acids in length can be accomplished by attaching the C-terminal amino acid of the sequence to an insoluble support followed by sequential addition of the remaining amino acids in the sequence. Techniques for solid phase synthesis are described by Barany & Merrifield, The Peptides: Analysis, Synthesis, Biology. Vol. 2: Special Methods in Peptide Synthesis, Part A. pp. 3-284; Merrifield et al., J. Am. Chem. Soc. 85:2149-2156, 1963, and Stewart et al., Solid Phase Peptide Synthesis, 2nd ed., Pierce Chem. Co., Rockford, Ill., 1984. Proteins of greater length may be synthesized by condensation of the amino and carboxyl termini of shorter fragments. Methods of forming peptide bonds by activation of a carboxyl terminal end (such as by the use of the coupling reagent N,N′-dicycylohexylcarbodimide) are known.

B. Inhibitory Nucleic Acid Molecules

Inhibitory nucleic acids that decrease the expression and/or activity of La protein can also be used in the methods disclosed herein. In some examples, such inhibitor nucleic acid molecules decrease La protein expression or activity by at least 20%, at least 40%, at least 50%, at least 75%, at least 80%, at least 90%, at least 95%, at least 98% or even 100%. One embodiment is an RNA interference (RNAi), such as, but not limited to, small inhibitory RNA (siRNA) or short hairpin RNA, which can be used for interference or inhibition of expression of a target. RNAs that specifically target La protein are commercially available, for example from Santa Cruz Biotechnology, Inc., ThermoFisher Scientific, and Sigma Aldrich. Exemplary commercially available RNAi sequences specific for La that can be used with the disclosed methods include:

Sense
(SEQ ID NO: 4)
GAAGCUAAAUUAAGAGCUAUU;
and
Antisense
(SEQ ID NO: 5)
UAGCUCUUAAUUUAGCUUCCA.

Generally, siRNAs are generated by the cleavage of relatively long double-stranded RNA molecules by Dicer or DCL enzymes (Zamore, Science, 296:1265-1269, 2002; Bernstein et al., Nature, 409:363-366, 2001). In animals and plants, siRNAs are assembled into RISC and guide the sequence specific ribonucleolytic activity of RISC, thereby resulting in the cleavage of mRNAs or other RNA target molecules in the cytoplasm. In the nucleus, siRNAs also guide heterochromatin-associated histone and DNA methylation, resulting in transcriptional silencing of individual genes or large chromatin domains.

The present disclosure provides RNA suitable for interference or inhibition of expression of La protein, which RNA includes double stranded RNA of about 19 to about 40 nucleotides with the sequence that is substantially identical to a portion of an mRNA or transcript of a target gene, such as La protein, for which interference or inhibition of expression is desired. For purposes of this disclosure, a sequence of the RNA “substantially identical” to a specific portion of the mRNA or transcript of the target gene for which interference or inhibition of expression is desired differs by no more than about 30%, and in some embodiments no more than about 10% or no more than 5% from the specific portion of the mRNA or transcript of the target gene. In particular embodiments, the sequence of the RNA is exactly identical to a specific portion of the mRNA or transcript of the target gene (e.g., La protein transcripts).

Thus, siRNAs disclosed herein include double-stranded RNA of about 15 to about 40 nucleotides in length and a 3′ or 5′ overhang having a length of 0 to 5-nucleotides on each strand, wherein the sequence of the double stranded RNA is substantially identical to (see above) a portion of a mRNA or transcript of a nucleic acid encoding La protein. In particular examples, the double stranded RNA contains about 19 to about 25 nucleotides, for instance 20, 21, or 22 nucleotides substantially identical to a nucleic acid encoding La protein. In additional examples, the double stranded RNA contains about 19 to about 25 nucleotides 100% identical to a nucleic acid encoding La protein. It should be noted that in this context “about” refers to integer amounts only. In one example, “about” 20 nucleotides refers to a nucleotide of 19 to 21 nucleotides in length.

Regarding the overhang on the double-stranded RNA, the length of the overhang is independent between the two strands, in that the length of one overhang is not dependent on the length of the overhang on other strand. In specific examples, the length of the 3′ or 5′ overhang is 0-nucleotide on at least one strand, and in some cases it is 0-nucleotide on both strands (thus, a blunt dsRNA). In other examples, the length of the 3′ or 5′ overhang is 1-nucleotide to 5-nucleotides on at least one strand. More particularly, in some examples the length of the 3′ or 5′ overhang is 2-nucleotides on at least one strand, or 2-nucleotides on both strands. In particular examples, the dsRNA molecule has 3′ overhangs of 2-nucleotides on both strands.

Thus, in one particular provided RNA embodiment, the double-stranded RNA contains 20, 21, or 22 nucleotides, and the length of the 3′ overhang is 2-nucleotides on both strands. In embodiments of the RNAs provided herein, the double-stranded RNA contains about 40-60% adenine+uracil (AU) and about 60-40% guanine+cytosine (GC). More particularly, in specific examples the double-stranded RNA contains about 50% AU and about 50% GC.

Also disclosed herein are RNAs that further include at least one modified ribonucleotide, for instance in the sense strand of the double-stranded RNA. In particular examples, the modified ribonucleotide is in the 3′ overhang of at least one strand, or more particularly in the 3′ overhang of the sense strand. It is contemplated that examples of modified ribonucleotides include ribonucleotides that include a detectable label (for instance, a fluorophore, such as rhodamine or FITC), a thiophosphate nucleotide analog, a deoxynucleotide (considered modified because the base molecule is ribonucleic acid), a 2′-fluorouracil, a 2′-aminouracil, a 2′-aminocytidine, a 4-thiouracil, a 5-bromouracil, a 5-iodouracil, a 5-(3-aminoallyl)-uracil, an inosine, or a 2′O-Me-nucleotide analog.

Antisense and ribozyme molecules for La protein are of use in the methods disclosed herein. Antisense nucleic acids are DNA or RNA molecules that are complementary to at least a portion of a specific mRNA molecule (Weintraub, Scientific American 262:40, 1990). In the cell, the antisense nucleic acids hybridize to the corresponding mRNA, forming a double-stranded molecule. The antisense nucleic acids interfere with the translation of the mRNA, since the cell will not translate an mRNA that is double-stranded. Antisense oligomers of about 15 nucleotides can be used, since they are easily synthesized and are less likely to cause problems than larger molecules when introduced into the target cell producing La protein. The use of antisense methods to inhibit the in vitro translation of genes is known (see, for example, Marcus-Sakura, Anal. Biochem. 172:289, 1988).

An antisense oligonucleotide can be, for example, about 5, 10, 15, 20, 25, 30, 35, 40, 45 or 50 nucleotides in length. An antisense nucleic acid can be constructed using chemical synthesis and enzymatic ligation reactions. For example, an antisense nucleic acid molecule can be chemically synthesized using naturally occurring nucleotides or variously modified nucleotides designed to increase the biological stability of the molecules or to increase the physical stability of the duplex formed between the antisense and sense nucleic acids, such as phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridin-e, 5-carboxymethylaminomethyluracil, dihydrouracil, beta-D-galactosylqueosine, inosine, amongst others.

Use of an oligonucleotide to stall transcription is known as the triplex strategy where an oligonucleotide winds around double-helical DNA, forming a three-strand helix. Therefore, these triplex compounds can be designed to recognize a unique site on a chosen gene (Maher, et al., Antisense Res. and Dev. 1(3):227, 1991; Helene, C., Anticancer Drug Design 6(6):569), 1991. This type of inhibitory oligonucleotide is also of use in the methods disclosed herein.

Ribozymes, which are RNA molecules possessing the ability to specifically cleave other single-stranded RNA in a manner analogous to DNA restriction endonucleases, are also of use. Through the modification of nucleotide sequences, which encode these RNAs, it is possible to engineer molecules that recognize specific nucleotide sequences in an RNA molecule and cleave it (Cech, J. Amer. Med. Assn. 260:3030, 1988). An advantage of this approach is that, because they are sequence-specific, only mRNAs with particular sequences are inactivated.

There are two basic types of ribozymes namely, Tetrahymena-type (Hasselhoff, Nature 334:585, 1988) and “hammerhead”-type. Tetrahymena-type ribozymes recognize sequences which are four bases in length, while “hammerhead”-type ribozymes recognize base sequences 11-18 bases in length. The longer the recognition sequence, the greater the likelihood that the sequence will occur exclusively in the target mRNA species. Consequently, hammerhead-type ribozymes are preferable to Tetrahymena-type ribozymes for inactivating a specific mRNA species and 18-base recognition sequences are preferable to shorter recognition sequences.

Various delivery systems are known and can be used to administer the siRNAs and other inhibitory nucleic acid molecules as therapeutics. Such systems include, for example, encapsulation in liposomes, microparticles, microcapsules, nanoparticles, recombinant cells capable of expressing the therapeutic molecule(s) (see, e.g., Wu et al., J. Biol. Chem. 262, 4429, 1987), construction of a therapeutic nucleic acid as part of a retroviral or other vector, and the like.

C. Chemical Compounds, Small Molecules and Caspase Inhibitors

La protein inhibitors include molecules that are identified from large libraries of both natural product or synthetic (or semi-synthetic) extracts or chemical libraries. Screening methods that detect decreases in La protein activity are useful for identifying compounds from a variety of sources for activity. The initial screens may be performed using a diverse library of compounds, a variety of other compounds and compound libraries. Thus, molecules that bind La protein, that inhibit the expression of La protein, and molecules that inhibit the activity of La protein can be identified. These small molecules can be identified from combinatorial libraries, natural product libraries, or other small molecule libraries. In addition, La antagonist can be identified as compounds from commercial sources, as well as commercially available analogs of identified inhibitors. The La antagonist can be tested, for example, in an assay to confirm it decreases osteoclast fusion.

The precise source of test extracts or compounds is not critical to the identification of La protein small molecule antagonists. Accordingly, La protein inhibitors can be identified from virtually any number of chemical extracts or compounds. Examples of such extracts or compounds that can be La protein inhibitors include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). La inhibitors can be identified from synthetic compound libraries that are commercially available from a number of companies including Maybridge Chemical Co. (Trevillet, Cornwall, UK), Comgenex (Princeton, N. J.), Brandon Associates (Merrimack, N.H.), and Microsource (New Milford, Conn.). La protein inhibitors can be identified from a rare chemical library, such as the library that is available from Aldrich (Milwaukee, Wis.). La protein inhibitors can be identified in libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). Natural and synthetically produced libraries and compounds are readily modified through conventional chemical, physical, and biochemical means.

Useful compounds that function as inhibitors can be found within numerous chemical classes, though typically they are organic compounds, including small organic compounds. Small organic compounds have a molecular weight of more than 50 yet less than about 2,500 daltons, such as less than about 750 or less than about 350 daltons can be utilized in the methods disclosed herein. Exemplary classes include heterocycles, peptides, saccharides, steroids, and the like. The compounds may be modified to enhance efficacy, stability, pharmaceutical compatibility, and the like.

In some embodiments, a caspase inhibitor is of use in the methods disclosed herein. Caspases are a family of intracellular endoproteases using a cysteine residue at the initiation of the cleavage of peptide substrates. The enzymatic properties of caspases are governed by the existence of a catalytic dyad (cysteine, histidine) where cysteine acts as a nucleophile for the initiation of cleavage of peptide bonds. The active site of Caspases is highly conserved, with the catalytic cysteine included in a peptide sequence QACXG (SEQ ID NO: 6) (where X is arginine (R), glutamine (Q) or glycine (G)) and a basic subsite SI, which gives them specificity for substrate cleavage after an aspartate residue, which is unique among mammalian proteases, except for the serine protease granzyme B. Generally, Caspases recognize a tetra-peptide motif, P1-P4 in N-ter of the cleavable bond, respectively recognized by subsites S1-S4 of the enzyme. The downstream positions Aspartate (P′1 and P′2) are also involved in the recognition and specificity of Caspases, see PCT Publication No. WO2017162674A1, incorporated herein by reference.

Caspases have been classified into three groups depending on the amino acid sequence that is preferred or primarily recognized. The group of caspases, which includes caspases 1, 4, and 5, has been shown to prefer hydrophobic aromatic amino acids at position 4 on the N-terminal side of the cleavage site. Another group which includes caspases 2, 3 and 7, recognize aspartyl residues at both positions 1 and 4 on the N-terminal side of the cleavage site, and preferably a sequence of Asp-Glu-X-Asp. A third group, which includes caspases 6, 8, 9 and 10, tolerate many amino acids in the primary recognition sequence, but seem to prefer residues with branched, aliphatic side chains such as valine and leucine at position 4. Additional information is provided for example, in PCT Publication No. WO2001010383A2, incorporated herein by reference in its entirety. Caspase inhibitors are also disclosed in Lee et al., Expert Opinion on Therapeutic Patents 28(1), DOI: 10.1080/13543776.2017.1378426, incorporated herein by reference. The caspase inhibitor can be a pan-caspase inhibitor, such as, but not limited to, including Q z-VAD-fmk, Q-VD-OPH OR Z-VKD-FMK, Emricasan or IDN-6556. In some embodiments, the caspase inhibitor is a pan-caspase inhibitor, such as caspase inhibitor z-VAD-fmk. Exemplary caspase inhibitors also include Z-DEVD-FMK, Ac-DMPD-CMK and Ac-DMLD-CMK, Ac-ATS010-KE, Rosmarinic acid and curcumin, Ac-DNLD-CHO, non-steroidal anti-inflammatory agents (NSAIDs), such as ibuprofen, naproxen, ketorolac, IDN-6556, Emricasan, GS 9450, and VRT-043198/VX-765 (Belnacasan). In some non-limiting examples, the caspase inhibitor can be a caspase 3, caspase 7 or caspase 8 inhibitor.

D. Inhibitory La Peptides

Peptide inhibitors of La are disclosed herein. These peptides bind La or a target molecule that specifically binds full length La protein and/or low molecular weight, cleaved La and inhibit the interaction of full-length La protein with the target. The peptide inhibitors can decrease osteoclast fusion in the subject and/or reduce bone resorption. These peptide inhibitors decrease La activity in the subject.

In some embodiments, these peptide inhibitors bind Annexin A5 and inhibit the interaction of full length La protein with Annexin A5. In further embodiments, the peptide inhibitors reduce osteoclast fusion as compared to a control, such as no treatment or treatment with vehicle alone.

In some embodiments, the peptide inhibitors include at least 15 amino acids of SEQ ID NO: 1, SEQ ID NO: 2, or SEQ ID NO: 7, and do not include the amino acid sequence of SEQ ID NO: 1, SEQ ID NO: 2, and/or SEQ ID NO: 7. The peptide inhibitors can include at least 15, 20, 25, 30, 35 or 40 amino acids of amino acids of SEQ ID NO: 1, SEQ ID NO: 2, or SEQ ID NO: 7, and do not include the amino acid sequence of SEQ ID NO: 1, SEQ ID NO: 2, and/or SEQ ID NO: 7. In more embodiments, the peptide inhibitors include 15-40 amino acids of SEQ ID NO: 1, SEQ ID NO: 2, or SEQ ID NO: 7, such as 20-40, 25-35, 25-40, or 30-40, 15-35, 15-30, or 15-25 amino acids of SEQ ID NO: 1, SEQ ID NO: 2, or SEQ ID NO: 7. In more embodiments, the peptide inhibitor is no more than 15, 20, 25, 30, 35 or 40 amino acids of SEQ ID NO: 1, SEQ ID NO: 2, or SEQ ID NO: 7. In yet other embodiments, the peptide inhibitor is 15, 20, 25, 30, 35 or 40 amino acids of SEQ ID NO: 1, SEQ ID NO: 2, or SEQ ID NO: 7. The peptide inhibitors inhibit osteoclast fusion induced by La protein. Suitable assays are disclosed for example, in the Examples section.

In some embodiments, the peptide inhibitors include at least 15 amino acids of an amino acid sequence a) having at least 95%, at least 96%, at least 97%, at least 98%, at least 99% or 100% sequence identity to SEQ ID NO: 1, SEQ ID NO: 2, or SEQ ID NO: 7, that promotes osteoclast fusion or b) having at most 15, 14, 13, 12, 11, 10, 9, 8, 7, 6, 5, 4, 3, 2 or 1 conservative substitutions in SEQ ID NO: 1, SEQ ID NO: 2, or SEQ ID NO:7. The peptide inhibitors can include at least 15, 20, 25, 30, 35 or 40 amino acids of an amino acid sequence a) having at least 95%, at least 96%, at least 97%, at least 98%, at least 99% or 100% sequence identity to SEQ ID NO: 1, SEQ ID NO: 2, or SEQ ID NO: 7, that promotes osteoclast fusion or b) having at most 15, 14, 13, 12, 11, 10, 9, 8, 7, 6, 5, 4, 3, 2 or 1 conservative substitutions in SEQ ID NO: 1, SEQ ID NO: 2, or SEQ ID NO:7. In more embodiments, the peptide inhibitors include 15-40 amino acids of an amino acid sequence a) having at least 95%, at least 96%, at least 97%, at least 98%, at least 99% or 100% sequence identity to SEQ ID NO: 1, SEQ ID NO: 2, or SEQ ID NO: 7, that promotes osteoclast fusion or b) having at most 15, 14, 13, 12, 11, 10, 9, 8, 7, 6, 5, 4, 3, 2 or 1 conservative substitutions in SEQ ID NO: 1, SEQ ID NO: 2, or SEQ ID NO:7. In more embodiments, the peptide inhibitor is no more than 15, 20, 25, 30, 35 or 40 amino acids of an amino acid sequence a) having at least 95%, at least 96%, at least 97%, at least 98%, at least 99% or 100% sequence identity to SEQ ID NO: 1, SEQ ID NO: 2, or SEQ ID NO: 7, that promotes osteoclast fusion or b) having at most 15, 14, 13, 12, 11, 10, 9, 8, 7, 6, 5, 4, 3, 2 or 1 conservative substitutions in SEQ ID NO: 1, SEQ ID NO: 2, or SEQ ID NO:7. In more embodiments, the peptide inhibitors include 15-40 amino acids of an amino acid sequence a) having at least 95%, at least 96%, at least 97%, at least 98%, at least 99% or 100% sequence identity to SEQ ID NO: 1, SEQ ID NO: 2, or SEQ ID NO: 7, that promotes osteoclast fusion or b) having at most 15, 14, 13, 12, 11, 10, 9, 8, 7, 6, 5, 4, 3, 2 or 1 conservative substitutions in SEQ ID NO: 1, SEQ ID NO: 2, or SEQ ID NO:7, such as 20-40, 25-40, or 30-40, 15-35, 15-30, or 15-25 amino acids of an amino acid sequence a) having at least 95%, at least 96%, at least 97%, at least 98%, at least 99% or 100% sequence identity to SEQ ID NO: 1, SEQ ID NO: 2, or SEQ ID NO: 7, that promotes osteoclast fusion or b) having at most 15, 14, 13, 12, 11, 10, 9, 8, 7, 6, 5, 4, 3, 2 or 1 conservative substitutions in SEQ ID NO: 1, SEQ ID NO: 2, or SEQ ID NO:7. In more embodiments, the peptide inhibitor is no more than 15, 20, 25, 30, 35 or 40 amino acids of an amino acid sequence a) having at least 95%, at least 96%, at least 97%, at least 98%, at least 99% or 100% sequence identity to SEQ ID NO: 1, SEQ ID NO: 2, or SEQ ID NO: 7, that promotes osteoclast fusion or b) having at most 15, 14, 13, 12, 11, 10, 9, 8, 7, 6, 5, 4, 3, 2 or 1 conservative substitutions in SEQ ID NO: 1, SEQ ID NO: 2, or SEQ ID NO:7. In yet other embodiments, the peptide inhibitor is 15, 20, 25, 30, 35 or 40 amino acids of an amino acid sequence a) having at least 95%, at least 96%, at least 97%, at least 98%, at least 99% or 100% sequence identity to SEQ ID NO: 1, SEQ ID NO: 2, or SEQ ID NO: 7, that promotes osteoclast fusion or b) having at most 15, 14, 13, 12, 11, 10, 9, 8, 7, 6, 5, 4, 3, 2 or 1 conservative substitutions in SEQ ID NO: 1, SEQ ID NO: 2, or SEQ ID NO:7. These peptide inhibitors inhibit osteoclast fusion induced by La protein. Suitable assays are disclosed for example, in the Examples section.

In specific non-limiting examples, the peptide inhibitor comprises, or consists of

Peptide 2:
(SEQ ID NO: 8)
AKLRAKQEQEAKQKLEEDAEMKSLEEKIGC;
or
Peptide 9:
(SEQ ID NO: 9)
GEVEKEALKKIIEDQQESLNKWKSKGRRFK.

In more embodiments, the peptide includes at least one amino acid substitution in either SEQ ID NO: 8 or SEQ ID NO: 9. In some embodiments, the peptide contains only a single amino acid substitution relative to SEQ ID NO: 8 or SEQ ID NO: 9. In other examples, the peptide includes two, three, four, five or more amino acid substitutions, such as two, three, four, five or more amino acid substitutions relative to SEQ ID NO: 8 or SEQ ID NO: 9. In further embodiments, the amino acid sequence of the peptide is at least 90%, at least 91%, at least 92%, at least 93%, at least 94%, at least 95% at least 96%, at least 97%, at least 98%, or at least 99% identical to SEQ ID NO: 8 or SEQ ID NO: 9. These peptide inhibitors inhibit osteoclast fusion induced by La protein. Suitable assays are disclosed for example, in the Examples section. In one embodiment, the peptide is at most 35 amino acids length. In some embodiments, the peptide is 25, 26, 27, 28, 29 or 30 amino acids in length. In one non-limiting example, the peptide is 30 amino acids in length. In specific non limiting examples, the inhibitory peptide: a) consists of SEQ ID NO: 8 or SEQ ID NO: 9; b) includes SEQ ID NO: 8 or SEQ ID NO: 9, and is at most 35 amino acids in length; c) consists of SEQ ID NO: 8 or SEQ ID NO: 9 with 1, 2, 3, 4 or 5 conservative substitutions; or d) includes SEQ ID NO: 8 or SEQ ID NO: 9 with 1, 2, 3, 4 or 5 conservative substitutions, and is at most 35 amino acids in length.

La peptide inhibitors of use can be prepared using recombinant methods, such as expression in host cells. Exemplary nucleic acid molecules can be prepared by cloning techniques, as disclosed above for La proteins. Nucleic acid molecules and vectors encoding a La peptide inhibitors are also of use in the disclosed methods, as disclosed above for La proteins. By introducing a nucleic acid and/or vector encoding a La peptide inhibitor, the activity of La protein is decreased in a subject. This aspect is disclosed above for the use of La protein, wherein the same expression control elements, vectors, and host cells can be used.

E. CRISR/Cas9

Included are in the present disclosure are methods for site-specific modification of a nucleic acid molecule in a cell (e.g., a genome, RNA). These modifications can include, but are not limited to, site-specific mutations, deletions, insertions, and replacements of nucleotides. These modifications can be made anywhere within the genome, for example, in genomic elements, including, among others, coding sequences, regulatory elements, and non-coding DNA sequences. Any number of such modifications can be made and the modifications may be made in any order or combination, for example, simultaneously, all together, or one after another. Such methods may be used to modify expression of a gene, such as La. Techniques for making such modifications by genome editing include, for example, use of CRISPR-Cas systems, zinc-finger nucleases (ZFNs), and transcription activator-like effector nucleases (TALENs), among others.

A typical set of CRISPR system is composed of two components, a CRISPR-associated nuclease 9 (Cas9) and one or more guide RNAs (gRNAs), each of which contains a CRISPR RNA (crRNA) and a trans-activating CRISPR RNA (tracrRNA). Simple gene disruptions can be generated by cleavage of the target site, followed by alteration of nucleic acids, such as a deletion, and repair by the non-homologous-end-joining pathway (NHEJ). Target recognition by crRNAs occurs through complementary base pairing with target DNA, which directs cleavage of foreign sequences by means of Cas proteins. In some embodiments, DNA recognition by guide RNA and consequent cleavage by the endonuclease requires complementary base-pairing with a protospacer adjacent motif (PAM) (e.g. 5′-NGG-3′) and with a protospacer region in the target. (Jinek et. al., Science. 337:816-821, 2012). The PAM motif recognized by a Cas9 varies for different Cas9 proteins. Any Cas9 protein can be used in the systems and methods disclosed herein. In other embodiments of the systems and methods disclosed herein, a promoter, is operably linked to the nucleic acid encoding Cas9. In one non-limiting example, a bone specific promoter is Runx2. [PLEASE SELECT PROMOTERS OF USE. WE BELIEVE THIS ONE IS BONE SPECIFIC, BUT MAY NOT BE FOR THE CORRECT CELLS.]

As noted above, the Cas9 RNA guide system includes a mature crRNA that is base-paired to trans-activating crRNA (tracrRNA), forming a two-RNA structure that directs Cas9 to the locus of a desired double-stranded (ds) break in target DNA, namely the gene encoding La. In some embodiments base-paired tracrRNA:crRNA combination is engineered as a single RNA chimera to produce a guide sequence (e.g., gRNA) which preserves the ability to direct sequence-specific Cas9 dsDNA cleavage (see Jinek et al., Science. 337:816-821, 2012). In some embodiments, the Cas9-guide sequence complex results in cleavage of one or both strands at a target sequence within the La gene. Thus, the Cas9 endonuclease (Jinek et al., Science. 337:816-821, 2012; Mali et. al., Nat Methods. 2013 October; 10(10): 1028-1034) and the gRNA molecules are used sequence-specific target recognition, cleavage, and genome editing of the La gene. In one embodiment, the cleavage site is at a specific nucleotide, such as, but not limited to the 16, 17, or 18^{th} nucleotide (nt) of a 20-nt target. In one non-limiting example, the cleavage site is at the 17^{th} nucleotide of a 20-nt target sequence. The cleavage can be a double stranded cleavage.

In some embodiments, the gRNA molecule is selected so that the target genomic targets bear a protospacer adjacent motif (PAM). In some embodiments, DNA recognition by guide RNA and consequent cleavage by the endonuclease requires the presence of a protospacer adjacent motif (PAM) (e.g., 5′-NGG-3′) in immediately after the target. The PAM is present in the targeted nucleic acid sequence but not in the crRNA that is produced to target it. In some embodiments, the proto-spacer adjacent motif (PAM) corresponds to 2 to 5 nucleotides starting immediately or in the vicinity of the proto-spacer at the leader distal end. The PAM motif also can be NNAGAA, NAG, NGGNG, AWG, CC, CC, CCN, TCN, or TTC.

In some embodiments, cleavage occurs at a site about 3 base-pairs upstream from the PAM. In some embodiments, the Cas9 nuclease cleaves a double stranded nucleic acid sequence.

In some embodiments, the guide sequence is selected to reduce the degree of secondary structure within the sequence. Secondary structure may be determined by any suitable polynucleotide folding algorithm. Some programs are based on calculating the minimal Gibbs free energy. An example of one such algorithm is mFold (Zuker and Stiegler, Nucleic Acids Res. 9 (1981), 133-148). Another example folding algorithm is the online webserver RNAfold, which uses the centroid structure prediction algorithm (see e.g., Gruber et al., 2008, Cell 106(1): 23-24; and Can and Church, 2009, Nature Biotechnology 27(12): 1151-62). Guide sequences can be designed using the MIT CRISPR design tool found at crispr.mit.edu, Harvard and University of Bergen CHOPCHOP web tool found at chopchop.cbu.uib.no, or the E-CRISP tool found at www.e-crisp.org/E-CRISP. Additional tools for designing tracrRNA and guide sequences are described in Naito et al., Bioinformatics. 2014 Nov. 20, and Ma et al. BioMed Research International, Volume 2013 (2013), Article ID 270805. The crRNA can be 18-48 nucleotides in length. The crRNA can be 18, 19, 20, 21, 22, 23, 24 or 25 nucleotides in length. In one example, the crRNA is 20 nucleotides in length.

The system disclosed herein introduces double stranded DNA breaks at the La gene, such that the La target is cleaved by Cas9. This results in functional La protein not being produced. In some embodiments, more than one DNA break can be introduced by using more than one gRNA. For example, two gRNAs can be utilized, such that two breaks are achieved. When two or more gRNAs are used to position two or more cleavage events, in a target nucleic acid, it is contemplated that in an embodiment the two or more cleavage events may be made by the same or different Cas9 proteins. For example, when two gRNAs are used to position two double strand breaks, a single Cas9 nuclease may be used to create both double strand breaks.

In some embodiments, the disclosed methods include the use of one or more vectors comprising: a) a bone specific promoter, such as Runx2 operably linked to a nucleotide sequence encoding a Type II Cas9 nuclease, b) a promoter, such as a U6 promoter, operably linked to one or more nucleotide sequences encoding one or more CRISPR-Cas guide RNAs that hybridize with the La gene in a target cell, such as a human cell. Components (a) and (b) can be located on same or different vectors, whereby the one or more guide RNAs target the La gene in the target cell and the Cas9 protein cleaves the La gene. In specific non-limiting examples, the one or more vectors are viral vectors such as lentiviral vectors. In other non-limiting examples, the viral vectors are adenovirus vectors, adeno-associated virus vectors, or retroviral vectors.

IV. Methods of Treatment and Pharmaceutical Compositions

Methods are disclosed herein for modulating osteoclast fusion. These methods can be practiced in vivo or in vitro. In some embodiments, the disclosed methods increase osteoclast fusion. In other embodiments, the disclosed methods decrease osteoclast fusion. When practiced in vivo, the disclosed methods can modulate bone resorption. In some embodiments, the disclosed methods increase bone resorption. In other embodiments, the disclosed methods decrease bone resorption. These methods include administering to the subject an effective amount of a La protein, a nucleic acid molecule encoding the La protein, or another agent that modulates La protein expression or activity.

The subject can be a human subject or a veterinary subject. The subject can be a mammal. While the disclosed methods will typically be used to treat human subjects, they may also be used to treat similar or identical diseases in other vertebrates, such as other primates, dogs, cats, horses, and cows.

Administration can be systemic or local. Examples of methods for administering the composition into mammals include, but are not limited to, oral, subcutaneous, intramuscular, intradermal, intraperitoneal, and intravenous administration. However, other routes, such as inhalation and rectal administration are contemplated. Local administration includes administration to a bone or joint of a subject. Generally, an effective amount modulates osteoclast fusion, as compared to a control, such as in the absence of treatment or treatment with a carrier.

In one embodiment, administration is local, such as to the perioseum. In another embodiment, administration is local such as by intramedullary injection. Intramedullary administration can be achieved by direct injection into the marrow space of the fracture site, without injection into the periosteum or bone cortex. Intramedullary administration can be administered by direct injection into the marrow, or by insertion of a K-wire through the intramedullary canal.

The administration can be systemic, to a subject in need thereof. In some embodiments, the effective amount increases osteoclast fusion in the subject. The effective amount can increase bone resorption in the subject. In other embodiments, the effective amount decreases osteoclast fusion in the subject. The effective amount can decrease bone resorption in the subject.

The administration can be local, such as to a bone of a subject in need thereof. In some embodiments, the effective amount increases osteoclast fusion in the subject. The effective amount can increase bone resorption in the subject. In other embodiments, the effective amount decreases osteoclast fusion in the subject. The effective amount can decrease bone resorption in the subject.

For the La protein, nucleic agent that encodes La protein, or another agent that modulates La protein expression or activity, single or multiple administrations can be administered depending on the dosage and frequency as required and tolerated by the subject. In any event, a composition should provide a sufficient quantity to effectively treat the subject, such as to modulate osteoclast fusion in the patient. The composition can be administered once but may be applied periodically until either a therapeutic result is achieved or until side effects warrant discontinuation of therapy. In one example, a dose is infused over time. In a one example, a continuous infusion is administered for about one to about ten days, such as for about two to five days, such as 1, 2, 3, 4, 5, 6, 7, 8, 9, or 10 days. In another example, a dose of an agent is administered as a bolus one or more times.

The subject can be treated at regular intervals, such as daily, biweekly, weekly, bi-monthly, or monthly, until a desired therapeutic result is achieved. Generally, the dose is sufficient to treat or ameliorate symptoms or signs of disease without producing unacceptable toxicity to the patient. Amounts effective for this use will depend upon the activity of the agent, the severity of the disease and the general state of the patient's health. An effective amount provides either subjective relief of a symptom(s) or an objectively identifiable improvement as noted by the clinician or other qualified observer. Combinations of agents are also envisioned. Administration may begin whenever the suppression or prevention of disease is desired, for example, at a certain age of a subject, or prior to an environmental exposure. In some embodiments, the method can modulate bone resorption in the subject in advance of appearance of one or more of the symptoms of a disease. In other embodiments, the method can modulate bone resorption in the subject after the onset of one or more symptoms of a disease.

For treatment of a subject, depending on activity of the agent, manner of administration, nature and severity of the disorder, age and body weight of the patient, different doses are necessary. Under certain circumstances, however, higher or lower doses may be appropriate. The administration of the dose can be carried out both by single administration in the form of an individual dose unit or else several smaller dose units and also by multiple administrations of subdivided doses at specific intervals. A skilled clinician can readily determine an effective dose.

For administration to a subject, an effective amount of La protein, a nucleic acid molecule encoding a La protein, or another agent that modulates La protein expression or activity, can be included in a pharmaceutically acceptable carrier. These pharmaceutical compositions can be prepared and administered in dose units. Solid dose units are tablets, capsules, single injectables and even suppositories. A suitable administration format may best be determined by a medical practitioner for each subject individually. Various pharmaceutically acceptable carriers and their formulation are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42: 2S, 1988. The dosage form of the pharmaceutical composition will be determined by the mode of administration chosen. Generally, the pharmaceutical compositions include an effective amount of La protein, or an agent that modulates La protein function or activity.

Suitable solid or liquid pharmaceutical preparation forms are, for example, granules, powders, tablets, coated tablets, (micro)capsules, suppositories, syrups, emulsions, suspensions, creams, aerosols, drops or injectable solution in ampoule form and also preparations with protracted release of active compounds, in whose preparation excipients and additives and/or auxiliaries such as disintegrants, binders, coating agents, swelling agents, lubricants, flavorings, sweeteners, solubilizers or scaffolds are customarily used as described above. The pharmaceutical compositions are suitable for use in a variety of drug delivery systems. For a brief review of present methods for drug delivery, see Langer, Science 249:1527-1533, 1990.

Controlled release parenteral formulations of the compositions can be made as implants, oily injections, or as particulate systems. For a broad overview of protein delivery systems see, Banga, A. J., Therapeutic Peptides and Proteins: Formulation, Processing, and Delivery Systems, Technomic Publishing Company, Inc., Lancaster, PA, (1995) incorporated herein by reference. Particulate systems include microspheres, microparticles, microcapsules, nanocapsules, nanospheres, and nanoparticles. Microcapsules contain the therapeutic protein, such as a cytotoxin or a drug, as a central core. In microspheres the therapeutic is dispersed throughout the particle. Particles, microspheres, and microcapsules smaller than about 1 pm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, e.g., Kreuter, J., Colloidal Drug Delivery Systems, J. Kreuter, ed., Marcel Dekker, Inc., New York, NY, pp. 219-342, 1994; and Tice & Tabibi, Treatise on Controlled Drug Delivery, A. Kydonieus, ed., Marcel Dekker, Inc. New York, NY, pp. 315-339, 1992, both of which are incorporated herein by reference.

To extend the time during which the agent is available, the therapeutic agent(s) can be provided as an implant, an oily injection, or as a particulate system. The particulate system can be a microparticle, a microcapsule, a microsphere, a nanocapsule, or similar particle.

Polymers can be used for ion-controlled release of the compositions disclosed herein. Various degradable and nondegradable polymeric matrices for use in controlled drug delivery are known in the art (Langer, Accounts Chem. Res. 26:537-542, 1993). For example, the block copolymer, polaxamer 407, exists as a viscous yet mobile liquid at low temperatures but forms a semisolid gel at body temperature. It has shown to be an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). Alternatively, hydroxyapatite has been used as a microcarrier for controlled release of proteins (Ijntema et al., Int. J. Pharm.112:215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, P A, 1993). Numerous additional systems for controlled delivery of therapeutic proteins are known. See, e.g., U.S. Pat. Nos. 5,055,303; 5,188,837; 4,235,871; 4,501,728; 4,837,028; 4,957,735; 5,019,369; 5,055,303; 5,514,670; 5,413,797; 5,268,164; 5,004,697; 4,902,505; 5,506,206; 5,271,961; 5,254,342 and 5,534,496, each of which is incorporated herein by reference.

In some embodiments, for local administration to bone, a scaffold is utilized, which includes, for example, a combination of polylactic acid and glycolic acid. By varying the proportion of the two components is polymers of different mechanical properties are obtained. Thus, in several embodiments, the ratio of polylactic acid: glycolic acid is about 1:1, about 2:1, about 3:1 or about 4:1. In one example, the scaffolding includes about 75% polylactic acid and about 25% glycolic acid.

In another embodiment, the scaffold is porous. For example, a scaffold can be about 85%, about 90%, about 95%, about 98% porous, such as for non-weight bearing tissue. In additional examples, the scaffold is about 5% porous, about 10% porous, about 15% porous or about 20% porous, such as for weight bearing tissue. The porosity can be determined, for example, by the fusing of micro spheres with CO₂ treatment. In this process commercial pellets of the polymer are converted to microspheres of the desired size, which are fused to develop a porous structure. By altering the micropore size scaffolds of different microporosity can be obtained. These scaffolds can be used, for example, with plasmid DNA, AAV viral vectors, transposon vectors and MLV vectors. Additional scaffolds are described above.

A. Additional Description of Methods for Increasing Osteoclast Fusion

In some embodiments, the method increases osteoclast fusion in the subject. These methods utilize a La protein, as disclosed above, a nucleic acid encoding the La protein, or a La protein agonist. In some embodiments, the subject has a disease that comprises reduced bone resorption. These include, but are not limited to, a bone fracture and osteoclast-poor osteopetrosis, genetic condition(s) of reduced osteoclastic bone resorption. These methods increase bone resorption and can be used for the treatment of disorders wherein increased bone resorption is beneficial to a subject. These subjects include, but are not limited to, a subject with a bone fracture and/or osteopetrosis. These methods can also influence spinal fusion.

Methods are provided to promote fracture healing. The fracture can be in any bone, including but not limited to cranial bones such as the frontal bone, parietal bone, temporal bone, occipital bone, sphenoid bone, ethmoid bone; facial bones such as the zygomatic bone, superior and inferior maxilla, nasal bone, mandible, palantine bone, lacrimal bone, vomer bone, the inferior nasal conchae; the bones of the ear, such as the malleus, incus, stapes; the hyoid bone; the bones of the shoulder, such as the clavicle or scapula; the bones of the thorax, such as the sternum or the ribs; the bones of the spinal column including the cervical vertebrae, lumbar vertebrae, and thoracic vertebrae; the bones of the arm, including the humerus, ulna and radius; the bones of the hands, including the scaphoid, lunate, triquetrum bone, psiform bone, trapezium bone, trapezoid bone, cpitate bone, and hamate bone; the bones of the palm such as the metacarpal bones; the bones of the fingers such as the proximal, intermediate and distal phalanges the bones of the pelvis such as the ilium, sacrum and coccyx; the bones of the legs, such as the femur, tibia, patella, and fibulal; the bones of the feet, such as the calcaneus, talus, navicular bone, medial cuneiform bone, intermediate cuniform bone, lateral cuneiform bone, cuboidal bone, metatarsal bone, proximal phalanges, intermediate phalanges and the distal phalanges; and the pelvic bones. In one example, a bone fracture is repaired in the absence of extra-skeletal bone formation, such as in the absence of bone formation in the soft tissues.

Methods are also provided to promote spinal fusion using the vectors described herein. Spinal fusion can be induced in any of the vertebrae, including, but not limited to, the cervical vertebrae, lumbar vertebrae, and thoracic vertebrae. In one example, spinal fusion occurs in the absence of extra-skeletal bone formation, such as in the absence of bone formation in the soft tissues.

In additional embodiments, the methods disclosed herein can be used to treat subjects that have a broken bone due to any disease, defect, or disorder which affects bone strength, function, and/or integrity, such as decreasing bone tensile strength and modulus. Examples of bone diseases include, but are not limited to, diseases of bone fragility, such as osteoporosis.

In some embodiments, a therapeutically effective dose is the quantity necessary to induce bone growth, increase the expression of prostaglandins, or to heal a fracture. The administration of the La protein, nucleic acid encoding the La protein, or other agent that increases La protein activity and/or expression, increases osteoclast fusion can arrest the symptoms of the fracture or spinal disorder, such as pain, and its complications in the subject. Amounts effective for this use will, of course, depend on the severity of the affliction and the weight and general state of the patient. Typically, dosages used in vitro may provide useful guidance in the amounts useful for in situ administration of the pharmaceutical composition, and animal models may be used to determine effective dosages for treatment of particular disorders. Various considerations are described, e.g., in Gilman et al., eds., Goodman And Gilman's: The Pharmacological Bases of Therapeutics, 8th ed., Pergamon Press, 1990; and Remington's Pharmaceutical Sciences, 17th ed., Mack Publishing Co., Easton, Pa., 1990, each of which is herein incorporated by reference.

Exemplary assays to determine if a method treats the bone defect include radiographic methods (Lehmann et al., Bone 35: 1247-1255, 2004; Rundle et al., Bone 32: 591-601, 2003; Nakamura et al., J. Bone Miner. Res. 13: 942-949, 1998); microcomputed tomography (pCT) methods (Nakamura et al., J. Bone Miner. Res. 13: 942-949, 1998; Lehmann et al., Bone 35: 1247-1255, 2004; Tamasi et al., J. Bone Miner. Res. 18: 1605-1611, 2003; Shefelbine et al., Bone 36:480-488, 2005); peripheral quantitative computed tomographic methods (Rundle et al., Bone 32: 591-601, 2003; Tamasi et al., J. Bone Miner. Res. 18: 1605-1611, 2003); dual energy X-ray absorptiometry methods (Holzer et al., Clin. Orthop. Rel. Res. 366: 258-263, 1999; Nakamura et al., J. Bone Miner. Res. 13: 42-949, 1998); histomorphometry methods (Lehmann et al., Bone 35: 247-1255, 2004; Tamasi et al., J. Bone Miner. Res. 18:1605-1611, 2003; Li et al., J. Bone Miner. Res. 17: 791-799, 2002; Schmidmaier et al., Bone 30: 816-822; 2002; Nakamura et al., J. Bone Miner. Res. 13:942-949, 1998; Sheng et al., Bone 30: 486-491, 2002); Masson's trichrome stain for collagen (Rundle et al., Bone 32: 591-601, 2003); Goldner's stain for collagen (Holzer et al., Clin. Orthop. Rel. Res. 366: 258-263; 1999); Von Kossa's silver stain for bone (Schmidmaier et al., Bone 30: 816-822, 2002); Safranin Orange stain for collagen (Schmidmaier et al., Bone 30: 816-822, 2002); and immunohistochemistry methods (Rundle et al., Bone 32: 591-601, 2003; Li et al., J. Bone Miner. Res. 17: 791-799, 2002; Safadi et al., J. Cell Physiol. 196: 51-62, 2003; Iwaki et al., J. Bone Miner. Res. 12: 96-102, 1997).

Treating a bone defect includes stimulation of bone formation which is sufficient to at least partially fill a void or structural discontinuity at the site of a bone defect. Treatment of the bone defect does not require a process of complete healing or a treatment which is 100% effective at restoring a defect to its pre-defect state. Successful treatment of a bone defect includes partial repair or healing, for example filling of at least 10%, at least 20%, at least 30%, at least 40%, at least 50%, at least 60%, at least 70%, at least 80%, at least 90%, at least 95%, or at least 99% of the bone defect with new bone material.

When a viral vector is utilized, it can be desirable to provide the recipient with a dosage of each recombinant virus in the composition of at least 10⁵, at least 10⁶, or at least 10⁷ plaque forming units/mg mammal, such as about 10⁵ to about 10¹⁰ plaque forming units/mg mammal, although a lower or higher dose can be administered. The composition of recombinant viral vectors can be introduced into a subject.

Generally, the quantity of recombinant viral vector, carrying the nucleic acid sequence of a polypeptide to be administered is based on the titer of virus particles. An exemplary range of the virus to be administered is 10⁵ to 10¹⁰ virus particles per mammal, such as a human.

In several embodiments, the La protein, nucleic acid encoding La protein, or agent that increases the activity and/or function of La protein can be administered in a conjunction with an additional therapeutic agent. Thus, for the treatment of a fracture, LMP-1, FGF-2, a BMP or related proteins, or a nucleic acid encoding one or more of these proteins can be administered. For example, LMP-1 is a transcription regulator that has been shown to induce bone formation by recruiting multiple bone morphogenic proteins (BMPs) (see Liu et al., Bone 35:673-681, 2004). Without being bound by theory, expression of LMP-1 induces cells to produce osteoinductive paracrine factors, such as prostaglandins and BMPs, which further enhance osteoblast differentiation in surrounding cells. In other embodiments, a bone morphogenic protein, such as BMP-2, BMP-4, BMP-7, and/or BMP-2/4 hybrid, or a nucleic acid encoding a bone morphogenic protein is administered. In another embodiment, a growth factor, such as FGF-2, is administered to further enhance fracture repair.

Other agents can also be administered, such as chemical compounds. In one embodiment, an anti-inflammatory agent, such as a non-steroidal anti-inflammatory agent, is administered to the subject. In another embodiment, an antibiotic, antifungal, or anti-viral agent is administered to the subject. Thus, other therapeutic agents can also be utilized in the disclosed methods, to promote fracture healing and/or spinal fusion.

When the subject has osteoporosis, the method can further include administering an effective amount of a bisphosphonate or calcitonin. In further embodiments, the method includes administering an effective amount of a bisphosphonate, an antibody that specifically binds receptor activator of nuclear factor kappa-B ligand (RANKL), and/or a teriparatide. In a specific non-limiting example, the antibody that specifically binds RANKL is denosumab. Examples of bisphosphonates include, but are not limited to, zoledronic acid, pamidronate, ibandronate, alendronate, and risedronate.

B. Additional Description of Methods that Decrease Osteoclast Fusion

In some embodiments, methods are disclosed for decreasing osteoclast fusion in the subject. The method incudes administering to the subject an effective amount of an agent that decreases La protein activity or expression in the subject. The method can decrease bone resorption in the subject. In further embodiments, the subject has a disease that comprises increased bone resorption. These methods decrease bone resorption and can be used for the treatment of disorders wherein increased bone resorption is observed in the subject. In some embodiments, the subject has osteoporosis, Paget's disease of bone, fibrous dysplasia, rheumatoid arthritis, osteomyelitis or metastatic bone disease. In one specific non-limiting example, the subject has fibrous dysplasia. In other embodiments, the subject has osteoclast-rich osteopetrosis.

In some embodiments the agent is an inhibitory nucleic acid molecule. In one specific, non-limiting example, a therapeutically effective amount of the polynucleotide is administered to a subject to treat a disease that comprises increased bone resorption.

In further embodiments, the agent is an inhibitory La peptide, a nucleic acid molecule encoding the inhibitory La peptide, or a vector include the nucleic acid molecule. The administration of an effective amount of the inhibitory La peptide, the nucleic acid molecule encoding the inhibitory La peptide, or a vector include the nucleic acid molecule, decreases osteoclast fusion in the subject.

Administration of nucleic acid constructs is taught, for example, in U.S. Pat. Nos. 5,643,578; 5,593,972 and 5,817,637; and 5,880,103. The methods include liposomal delivery of the nucleic acids (or of the La protein).

One approach to administration of nucleic acids is direct administration of plasmid DNA, such as with a mammalian expression plasmid. As described above, the nucleotide sequence encoding a polypeptide can be placed under the control of a promoter to increase expression of the molecule. A CRISPR/Cas system is also of use in the methods disclosed herein.

The inhibitory nucleic acid molecule can also be expressed by attenuated viral hosts or vectors or bacterial vectors. Recombinant vaccinia virus, adeno-associated virus (AAV), herpes virus, retrovirus, or other viral vectors can be used to express the peptide or protein. For example, vaccinia vectors and methods of administration are described in U.S. Pat. No. 4,722,848. BCG (Bacillus Calmette Guerin) provides another vector for expression of the peptides (see Stover, Nature 351:456-460, 1991).

When a viral vector is utilized, it can be desirable to provide the recipient with a dosage of each recombinant virus in the composition of at least 10⁵, at least 10⁶, or at least 10⁷ plaque forming units/mg mammal, such as about 10⁵ to about 10¹⁰ plaque forming units/mg mammal, although a lower or higher dose can be administered. The composition of recombinant viral vectors can be introduced into a subject. Generally, the quantity of recombinant viral vector to be administered is based on the titer of virus particles. An exemplary range of the virus to be administered is 10⁵ to 10¹⁰ virus particles per mammal, such as a human.

In some embodiments, and effective amount of an antagonistic antibody or antigen binding fragment thereof is administered to the subject. Antagonistic antibodies and antigen binding fragments thereof can be provided in lyophilized form and rehydrated with sterile water before administration, although they are also provided in sterile solutions of known concentration. The antibody solution is then added to an infusion bag containing 0.9% Sodium Chloride, USP, and typically administered at a dosage of from 0.5 to 15 mg/kg of body weight. Considerable experience is available in the art in the administration of antibody drugs, which have been marketed in the U.S. since the approval of RITUXAN® in 1997. Antibody drugs can be administered by slow infusion, rather than in an IV push or bolus. In one example, a higher loading dose is administered, with subsequent, maintenance doses being administered at a lower level. For example, an initial loading dose of at least 0.5 mg/kg, such as at least 1 mg/kg, such as 4 mg/kg may be infused over a period of some 90 minutes, followed by weekly maintenance doses for 4-8 weeks of at least 0.5 mg/kg, such as at least 1 mg/kg, such as 2 mg/kg infused over a 30-minute period if the previous dose was well tolerated.

In one specific, non-limiting example, a pharmaceutical composition for intravenous administration includes about 0.1 μg to 10 mg of an antagonist antibody, or antigen binding fragment thereof, per patient per day. Dosages from 0.1 up to about 100 mg per subject per day can be used, particularly if the antagonistic antibody or antigen binding fragment thereof is administered to a body cavity or into a lumen of an organ.

In some embodiments, and additional agent is administered to the subject. In some embodiments, the subject has osteoporosis. In further embodiments, the method includes administering an effective amount of a bisphosphonate, an antibody that specifically binds receptor activator of nuclear factor kappa-B ligand (RANKL), and/or a teriparatide. In a specific non-limiting example, the antibody that specifically binds RANKL is denosumab. Examples of bisphosphonates include, but are not limited to, zoledronic acid, pamidronate, ibandronate, alendronate, and risedronate.

In some embodiments, the subject has Paget's disease of bone. The method can include administering an effective amount of a bisphosphonate or calcitonin. Examples of bisphosphonates include, but are not limited to, zoledronic acid, pamidronate, ibandronate, alendronate, and risedronate.

In more embodiments the subject has fibrous dysplasia. The method can include administering denosumab (Boyce et al., J Bone Miner Res. 2012 July; 27(7): 1462-1470.). The method can include administering an effective amount of a bisphosphonate. Examples of bisphosphonates include, but are not limited to, zoledronic acid, pamidronate, ibandronate, alendronate, and risedronate.

In further embodiments, the subject has rheumatoid arthritis. The method can include administering an effective amount of a non-steroidal anti-inflammatory agent, a steroid, methotrexate, leflunomide, hydroxychloroquine, sulfasalazine, tofacitinib, abatacept, adalimumab, anakinara, baricitinib, certolizumab, entanercept, golimumab, infliximab, rituximab, sarilumab, and/or tocilzumab.

In more embodiments, the subject has osteomyelitis. The method can include administering an effective amount of an antibiotic. Suitable antibiotics include, but are not limit to, amoxicillin-clavulanate, ciprofloxacin plus clindamycin, Levofloxacin plus clindamycin or moxifloxacin). The method can also include administering an anti-fungal, such as, but not limited to, itraconazole, fluconazole, ketoconazole, terbinafine, andvoriconazole. The method can also include administering denosumab and/or teriparatide.

In yet other embodiments, the subject has metastatic bone disease. In further embodiments, the subject has an osteosarcoma. The method can include treating the subject with a chemotherapeutic agent, immunotherapy or radiation. The primary cancer can be, for example, a breast tumor, lung tumor, thyroid tumor, kidney tumor, or a prostate tumor. The method can include administering an effective amount of a bisphosphonate or calcitonin. Examples of bisphosphonates include, but are not limited to, Zoledronic acid, pamidronate, Ibandronate, Alendronate, and Risedronate. The method can also include administering denosumab.

V. Kits

Kits are also provided. In some embodiments, the kits can include La protein, a fragment thereof. The kit can also include nucleic acids or vectors encoding La protein. In other embodiments, the kit can include an agent that decreases La protein expression or activity in the subject.

In some embodiments, the kit includes a) La protein or an effective fragment thereof, or a nucleic acid molecule or vector encoding the La protein or effective fragment thereof, optionally b) one or more proteins which interact with La and can modulate its activity, or effective fragments thereof, or a nucleic acid molecule or vector encoding these proteins or effective fragment thereof, and c) LMP-1, FGF-2, a BMP or related proteins, or a nucleic acid encoding one or more of these proteins. In some embodiments, the kit includes elements a) and c).

In more embodiments, the kit includes a) an agent that decreases La protein expression or activity in the subject; and b) a bisphosphonate, an antibody that specifically binds RANKL, and/or a teriparatide. In further embodiments, the agent that decreases La protein expression or activity in the subject is i) an inhibitory nucleic acid molecule, ii) an antagonistic antibody that specifically binds the La protein; iii) a caspase inhibitor, or iv) an inhibitory peptide or a nucleic acid molecule encoding the inhibitory peptide. In some non-limiting examples, the inhibitory peptide includes or consists of SEQ ID NO: 8 or SEQ ID NO: 9 SEQ ID NO: 8 or SEQ ID NO: 9, or includes or consists of SEQ ID NO: 8 or SEQ ID NO: 9 with 1, 2, 3, 4 or 5 conservative substitutions. In further embodiments, the kit includes a CRISPR nuclease or a nucleic acid molecule encoding the CRISPR nuclease.

The kits may also include additional components to facilitate the particular application for which the kit is designed. For example, kits may additionally include buffers and other reagents routinely used for the practice of a particular method.

The kit can include a container and a label or package insert on or associated with the container. Suitable containers include, for example, bottles, vials, syringes, etc. The containers may be formed from a variety of materials such as glass or plastic. In several embodiments, the container may have an access port (for example the container may be an intravenous solution bag or a vial having a stopper pierceable by a hypodermic injection needle) so that a specific amount of an agent can be withdrawn.

A label or package insert indicates the use of the composition(s). The package insert typically includes instructions customarily included in commercial packages of products that contain information about the indications, dosages, contraindications and/or warnings concerning the use of such products. The instructional materials may be written, in an electronic form (such as a computer diskette or compact disk) or may be visual (such as video files).

This disclosure is illustrated by the following non-limiting examples:

### EXAMPLES

It is disclosed herein that osteoclast formation is accompanied by, and depends on, drastic changes in the steady-state level, molecular species, and intracellular localization of La protein. It is demonstrated that La functions as a regulator of osteoclast fusion and impacts osteoclasts' ability to resorb bone. Surprisingly, La, present in primary human monocytes, nearly disappears in M-CSF derived osteoclast precursors. RANKL-induced commitment to osteoclastogenesis drives the reappearance of La protein in a cleaved form at the surface of committed, fusing osteoclasts. As osteoclast fusion plateaus, cleaved La disappears and higher molecular weight, full-length protein (FL-La) is observed within the nuclei of mature, multinucleated osteoclasts. Perturbing La expression, cleavage or surface function inhibits osteoclast fusion, while exogenous, surface La promotes fusion. Moreover, the mechanism by which La promotes osteoclast fusion is independent of La's ability to interact with RNA through its highly conserved La domain. Indeed, a C-terminal portion of La (SEQ ID NO: 7), lacking the La domain and RNA recognition motif 1 (RRM1) is sufficient to promote fusion between human osteoclasts. The findings indicate that La protein has been adapted in mammals to serve as an osteoclast fusion manager. Thus, La is a target for treatment of bone diseases stemming from perturbed bone turnover.

### Example 1

### Formation of Multinucleated Osteoclasts Involves La Protein

It was demonstrated that osteoclastogenesis is accompanied by drastic changes in La levels, molecular species and location within fusing osteoclasts. A cleaved, non-nuclear La species promotes osteoclast formation, and as cells arrive at a mature size, LMW La is replaced by FL La detected in the nuclei of syncytial osteoclasts.

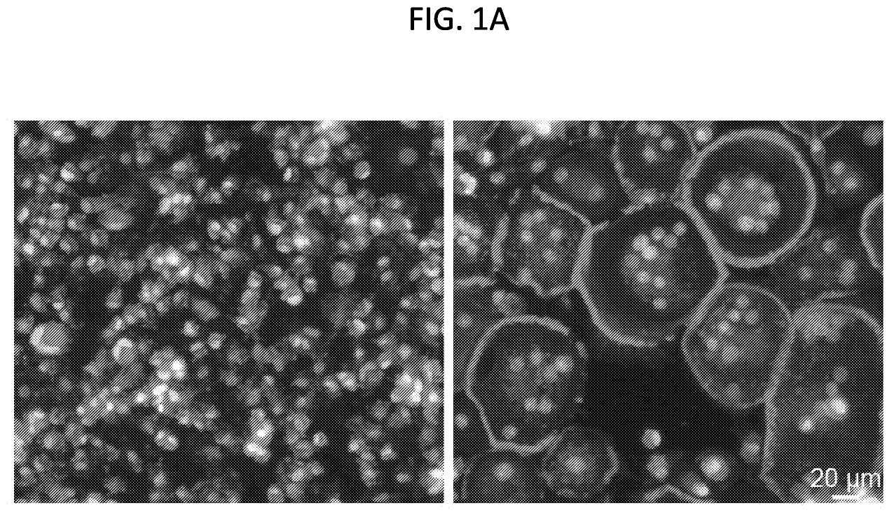

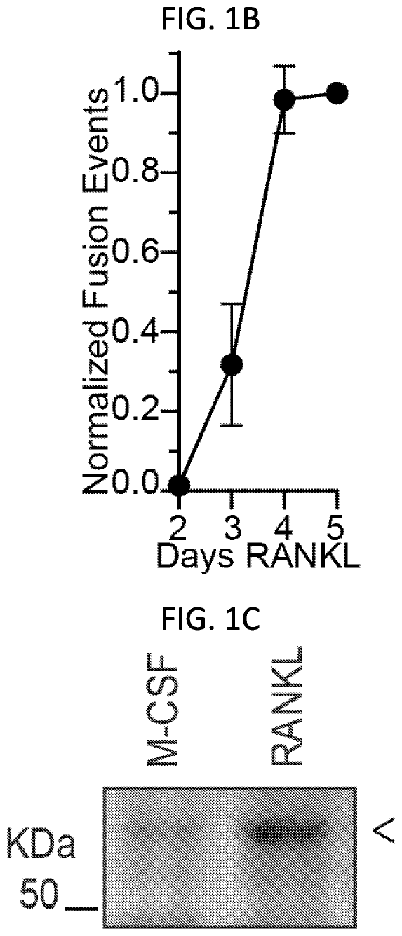

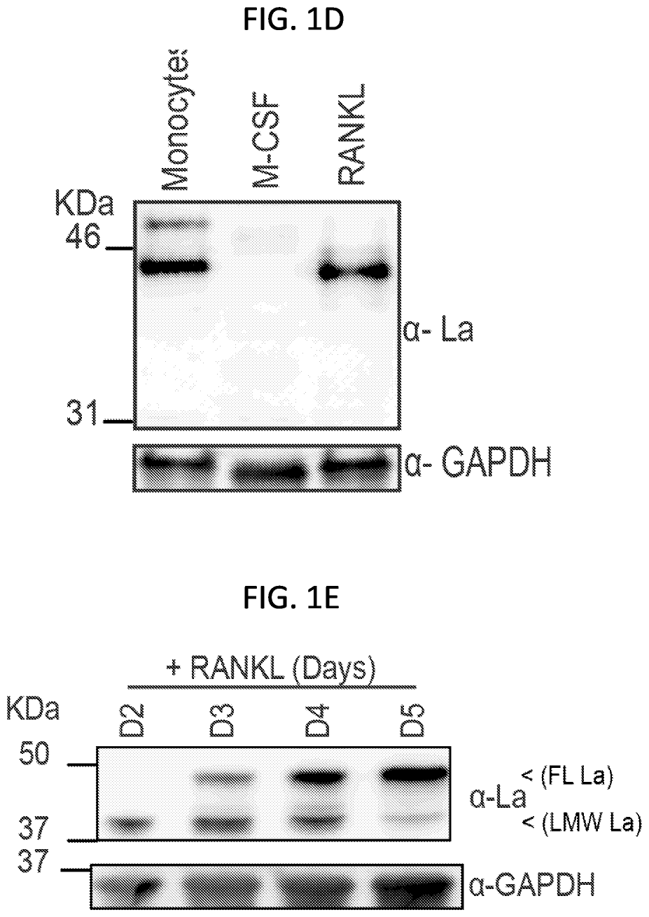

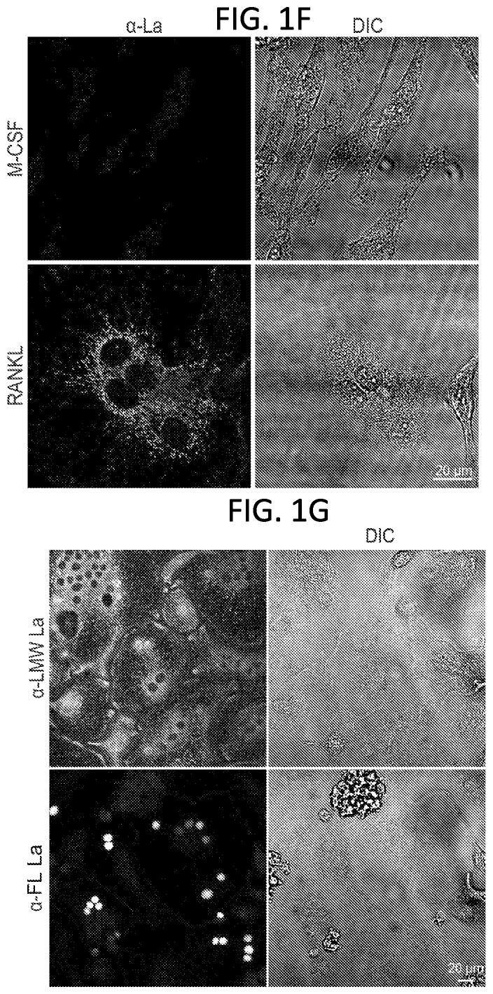

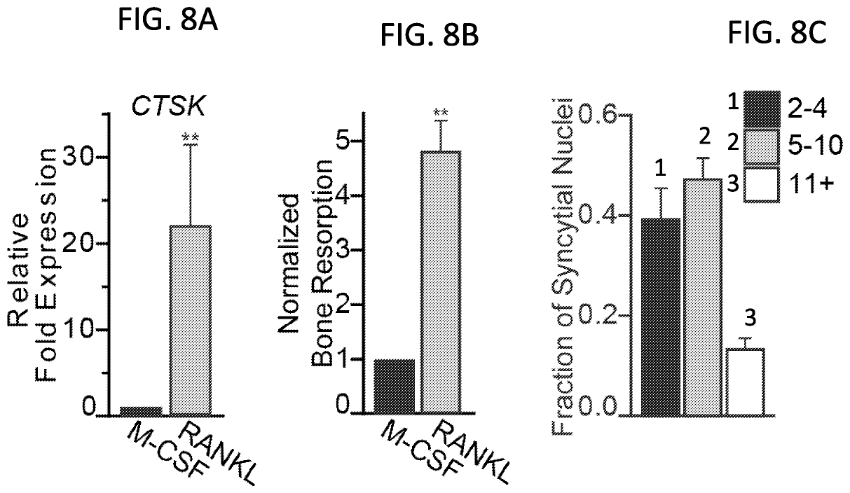

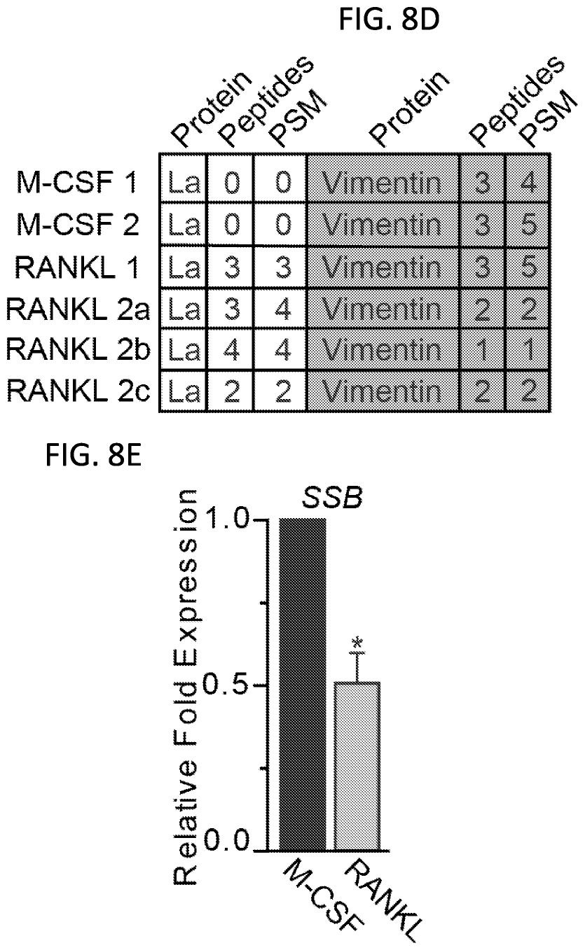

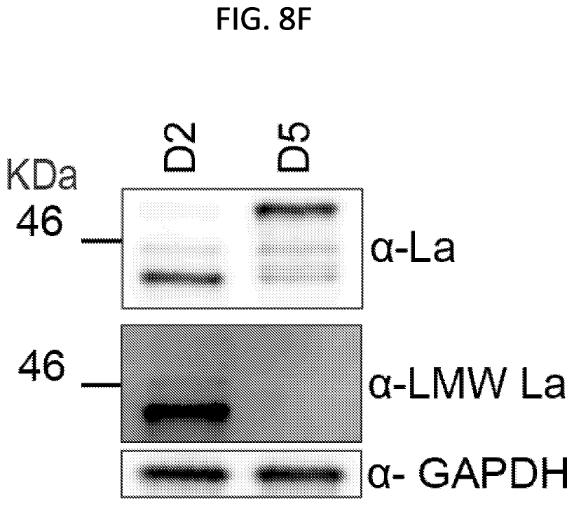

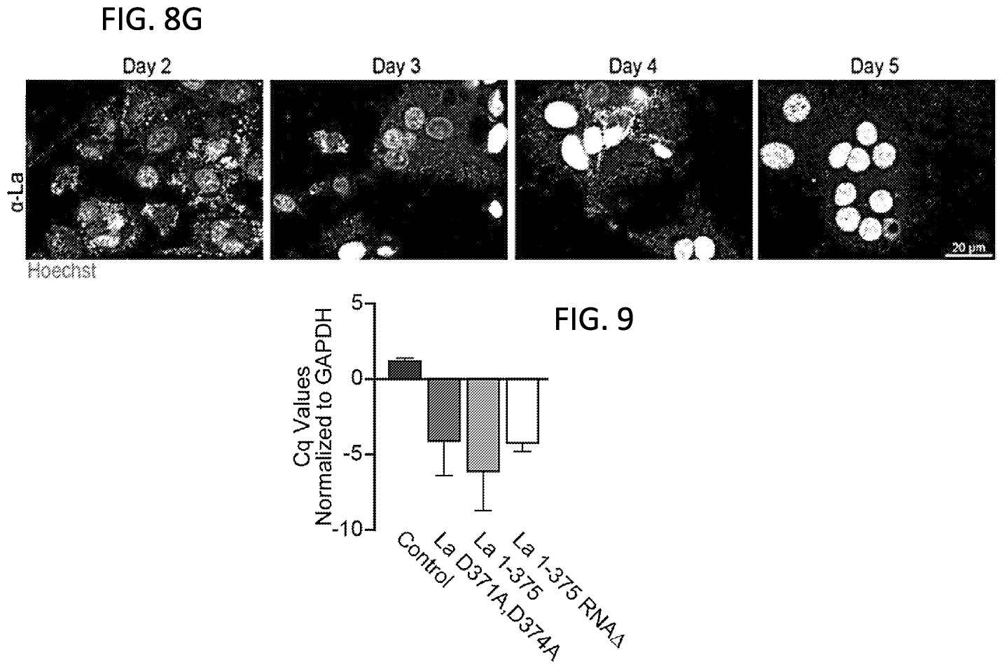

Human osteoclastogenesis was modeled by treating primary monocytes with M-CSF to derive mononucleated osteoclast precursors to which recombinant RANKL was later added to obtain multinucleated osteoclasts that readily resorb bone (FIGS. 1A, 1B, FIGS. 8A-8C) (Verma et al., J Biol Chem 293, 254-270, doi:10.1074/jbc.M117.809681 (2018)). Osteoclast precursors begin fusing at ˜2 days following RANKL addition and after ˜5 days reach the sizes (˜5-10 nuclei/cell) characteristic of mature multinucleated osteoclasts^{10,35,36}(FIG. 1B, FIG. 8C) (Moller et al., Int J Mol Sci 21, doi:10.3390/ijms21176368 (2020); Abdallah et al., Front Immunol 9, 632, doi:10.3389/fimmu.2018.00632 (2018); Stattin et al., Sci Rep 7, 3012, doi:10.1038/s41598-017-02533-2 (2017)).

While evaluating proteomic changes associated with osteoclastogenesis, a distinct protein was discovered that was nearly absent in M-CSF derived precursors but abundantly expressed in osteoclasts following ˜3 days of RANKL stimulated osteoclastogenesis, when the cells were rapidly fusing (FIG. 1C, arrow). Using mass spectrometry analysis, this protein was identified as La (FIG. 8D). The low level of La in M-CSF-derived macrophages was unexpected, as La is generally considered an abundant, ubiquitous protein (see, for example, Wolin and Cedervall, Annu Rev Biochem 71, 375-403, doi:10.1146/annurev.biochem.71.090501.150003 (2002)).

Western blot analysis confirmed that La is well expressed in monocytes, markedly reduced in M-CSF-derived osteoclast precursors and La's high steady-state levels return during RANKL-induced osteoclast formation (FIG. 1D). The data suggested that La's tight regulation during osteoclastogenesis was likely carried postranslationally, as M-CSF derived precursors contained even more La transcript (gene SSB) than after RANKL application (FIG. 8E). La appeared as two distinct, temporally separated molecular species during osteoclastogenesis (FIG. 1E). A low molecular weight (LMW La) species detected at the timepoints at the onset and during robust fusion was replaced by a higher molecular weight species, corresponding to full-length La (FL La), as fusion slows and osteoclasts reach a mature size.

In addition to changes in molecular weight, osteoclastogenic differentiation of human monocytes is accompanied by a dramatic change in La's location within cells. Canonically, La exhibits robust nuclear staining (Wolin and Cedervall, Annu Rev Biochem 71, 375-403, doi:10.1146/annurev.biochem.71.090501.150003 (2002)), as illustrated for HeLa cells. In contrast, M-CSF derived osteoclast precursors exhibited minimal La staining (FIG. 1F), consistent with the biochemical analysis (FIG. 1D). Addition of RANKL produced abundant La signal in committed, fusing osteoclasts, however, in contrast to other human cell types and tissues (Wolin and Cedervall, Annu Rev Biochem 71, 375-403, doi:10.1 146/annurev.biochem.71.090501.150003 (2002); Maraia et al., Wiley Interdiscip Rev RNA 8, doi:10.1002/wrna.1430 (2017)), La appeared as distinct, predominantly non-nuclear puncta throughout fusogenic osteoclasts during early stages of osteoclast fusion (FIG. 1F).

The anti-La antibody (α-La) used in FIG. 1D recognizes both LMW La and FL La species. To determine whether the different molecular weight species of La exhibit different localization during osteoclastogenesis, several commercially available antibodies were analyzed, and antibodies were selected that preferentially recognized LMW La species (α-LMW La, see also FIG. 8F and the Table below).

TABLE
Antibodies used for the detection of La molecular species in
human osteoclasts.

| | | La Species Recognized in |
|---|---|---|
| Antibody Producer/# | Referred to as | Human Osteoclasts |
| Abcam/75927 | α-La | LMW La and FL La |
| Invitrogen/PA5-29763 | α-LMW La | LMW La |
| Abcam/61800 | a-FL La | PhosphoSer366 FL La |

FL La is largely phosphorylated at Ser366 and localizes to the nucleus (Intine et al., Mol Cell 12, 1301-1307, doi:10.1016/s1097-2765(03)00429-5 (2003)). Previous work has demonstrated that LMW La is produced by the cleavage of FL La, but that FL La must be dephosphorylated at 366 before FL La can be cleaved 33. Moreover, previous reports have shown that antibodies specific for phosphoSer366 La do not recognize LMW La (Rutjes et al., Cell Death Differ 6, 976-986, doi:10.1038/sj.cdd.4400571 (1999)). For these experiments, α-phosphoSer366 La antibodies was used to preferentially stain for FL La (α-FL La). At intermediate timepoints, where both La molecular species were present, it was found that LMW La was predominantly detected outside the nucleus, throughout the cell, while FL La was observed exclusively in the nuclei of fused cells (FIG. 1G). The shift in La distribution during osteoclastogenesis was further confirmed by staining with α-La at different days post RANKL application (FIG. 8G).

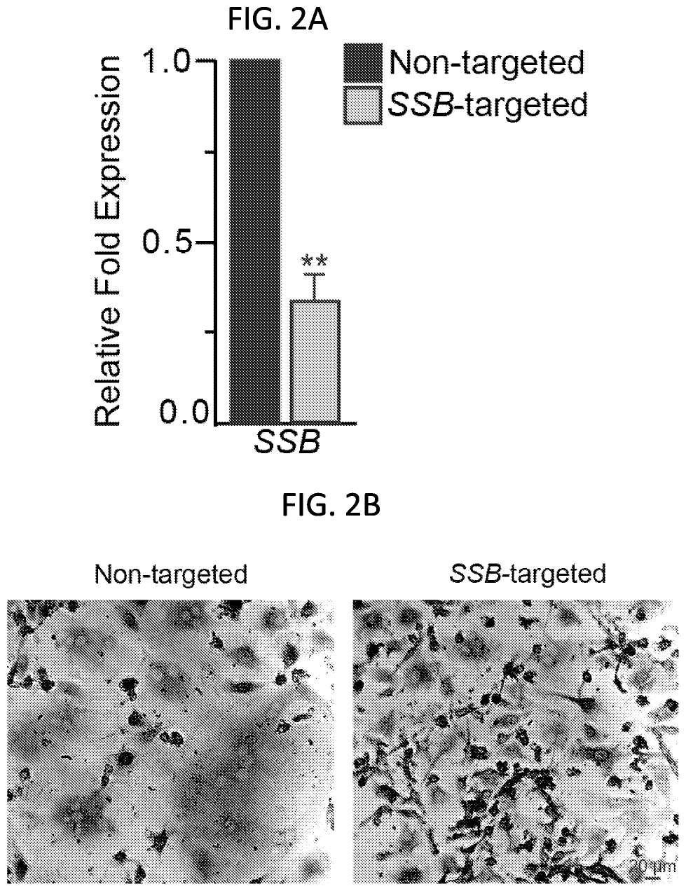

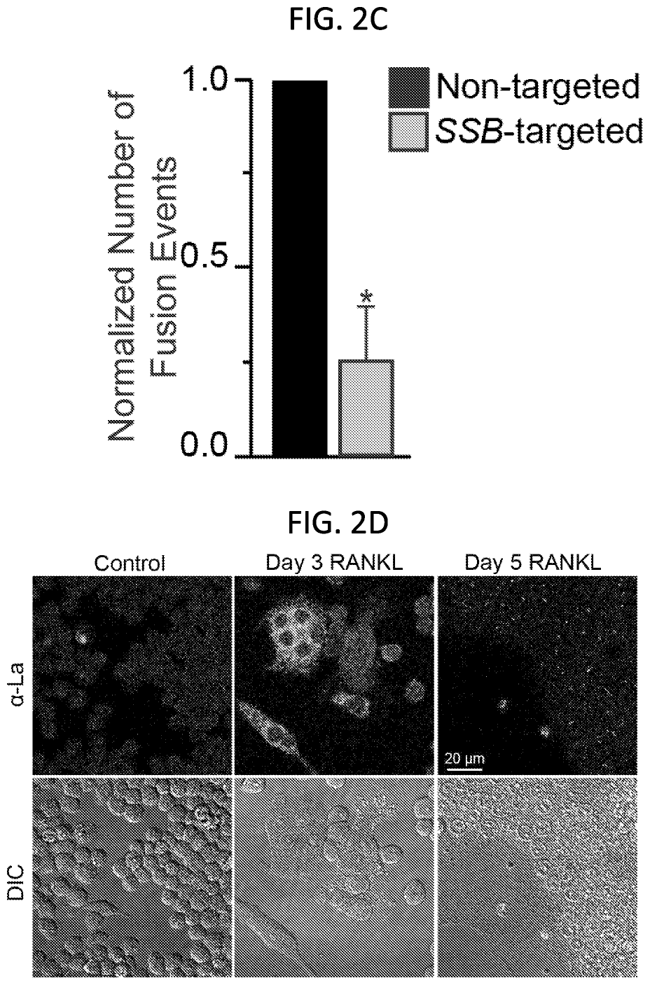

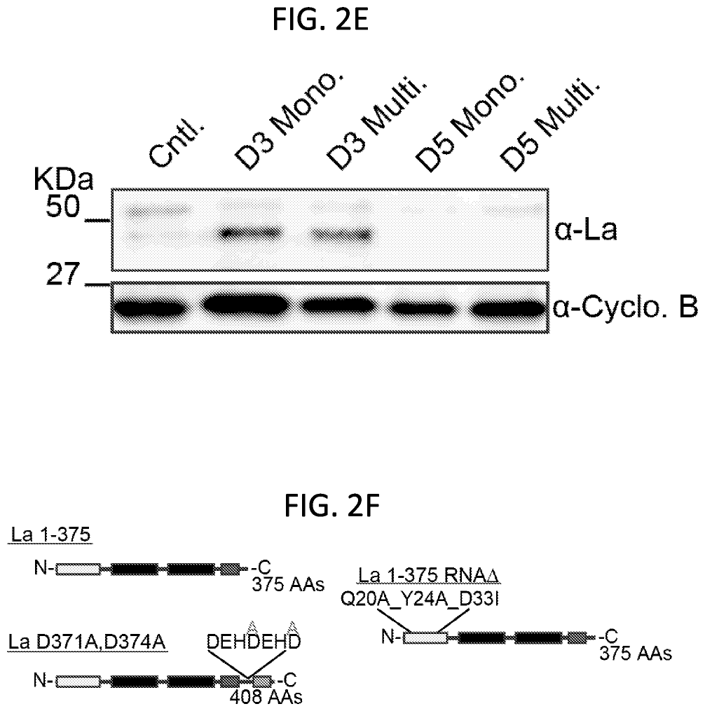

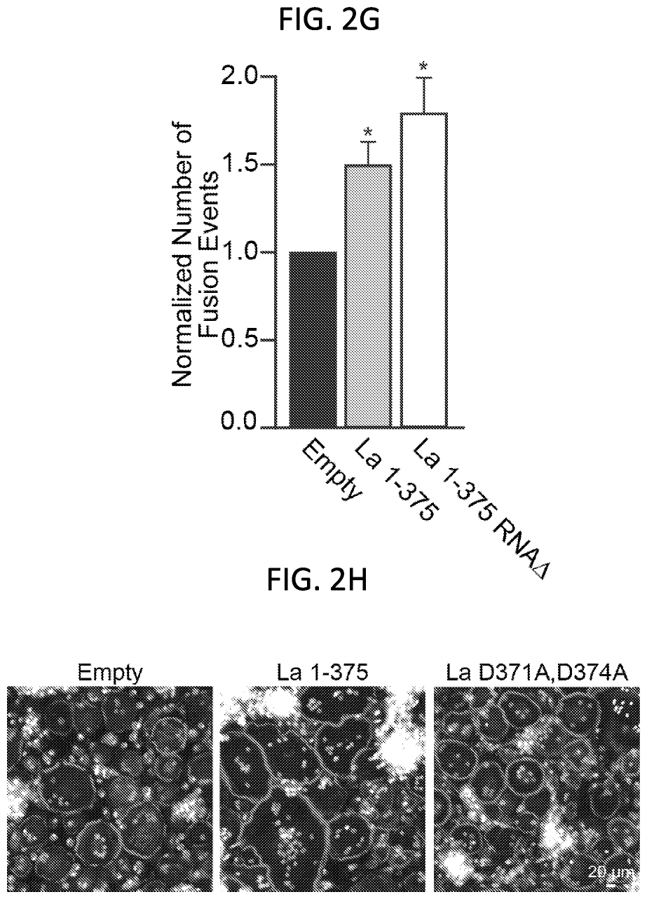

It was found that osteoclastogenic differentiation is accompanied by drastic changes in the expression and localization of La motivated. It was determined whether La is functionally involved in osteoclast formation. It was found that La expression tightly regulates the formation of multinucleated osteoclasts. RNAi mediated reduction of La transcript (SSB) drastically inhibited osteoclast fusion (FIG. 2A-2C). Cytosolic localization of La at the time of fusion was also observed during osteoclastogenic differentiation and fusion of RAW 264.7 derived murine osteoclast precursors (FIG. 2D). Furthermore, Western blot analysis of cell lysates collected separately from mostly mononucleated cells and from mostly multinucleated cells indicated that the robust fusion at day 3 post-RANKL was accompanied by a drastic increase in steady-state levels of La (FIG. 2E). These findings suggested that La dependence in osteoclast formation is conserved in humans and mice. Note that in distinction to human cells, in the case of RAW 264.7 cells, following the stage of active fusion by day 5, the levels of La returned to lower pre-fusion levels.

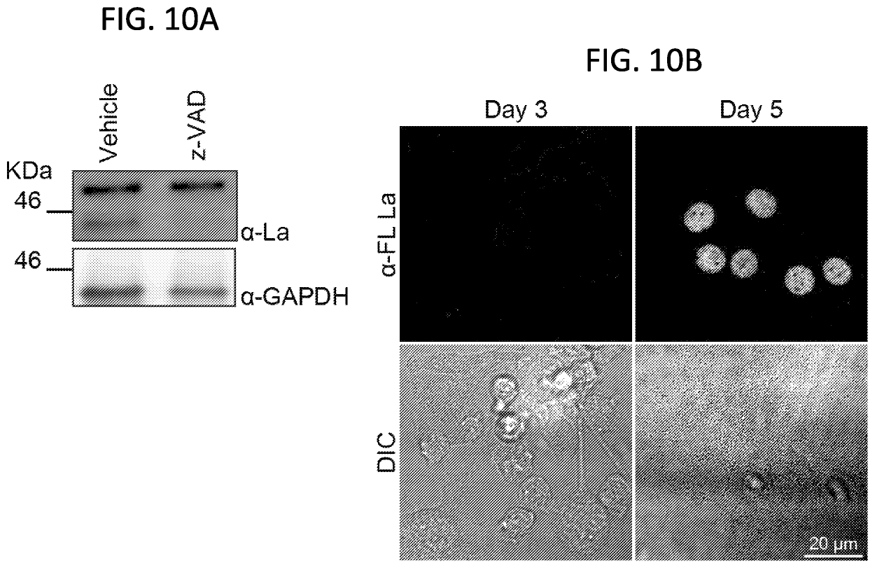

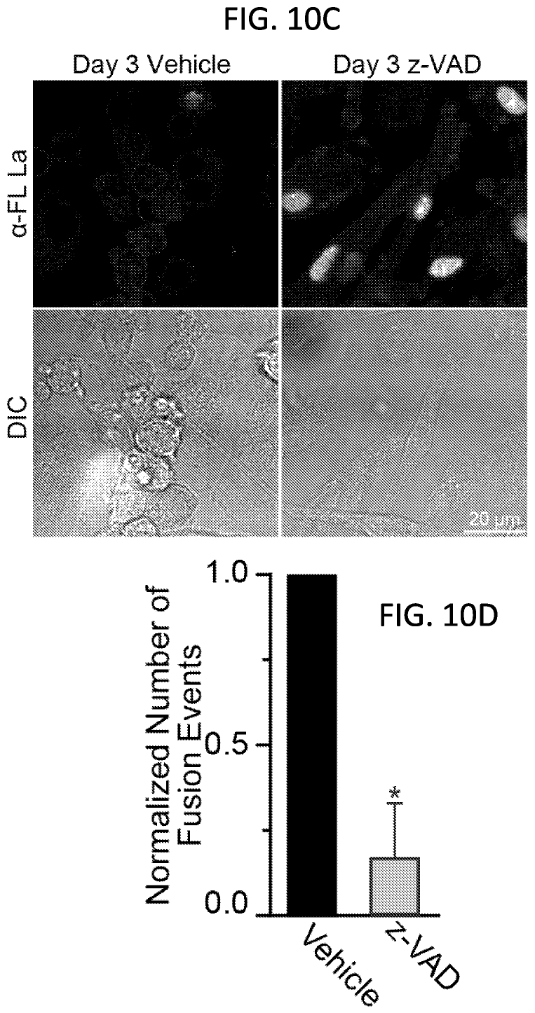

To identify the functional form of La associated with osteoclastogenesis, the relationship was determined between the appearance of LMW La and fusion between osteoclasts. During apoptotic progression human La is cleaved by caspases at Glu-375, removing La's NLS (Rutjes et al., Cell Death Differ 6, 976-986, doi:10.1038/sj.cdd.4400571 (1999); Ayukawa et al., J Biol Chem 275, 34465-34470, doi:10.1074/jbc.M003673200 (2000)). It was found that overexpression of La 1-375, mimicking this cleaved species, greatly promoted fusion in both RAW 264.7 derived, murine osteoclasts and monocyte derived, human osteoclasts (FIG. 2F-2I). In contrast, an uncleavable mutant of FL La, D371A,D374A La (point mutations disrupting La's predicted caspase cleavage sites) had no effect on osteoclast fusion despite being expressed at similar levels, suggesting that formation of multinucleated osteoclasts depends on LMW La (FIG. 2H, 2I and FIG. 9). It was also found that the pan caspase inhibitor z-VAD blocked the production of LMW La in differentiating osteoclasts (FIG. 10A). Blocking the caspase-dependent production of LMW La resulted in the premature retention of FL La within the nuclei of unfused osteoclasts (FIG. 10C vs 10B) and significantly perturbed the ability of osteoclasts to form multinucleated syncytia (FIG. 10D), see also Szymczyk et al., J Cell Physiol 209, 836-844, doi:10.1002/jcp.20770 (2006). This data further supports the role of caspase-cleaved, LMW La in the formation of multinucleated osteoclasts.

### Example 2

### Cell Surface Associated La Regulates the Cell Fusion Stage of Osteoclast Formation

It was demonstrated that, while on its own La has no fusogenic activity, at the surface of differentiating osteoclasts it promotes fusion of their membranes. This promotion does not require La domain, RRM1 or the NLS of La and may involve complexes that La forms with Anx A5, one of the components of the osteoclast fusion machinery.

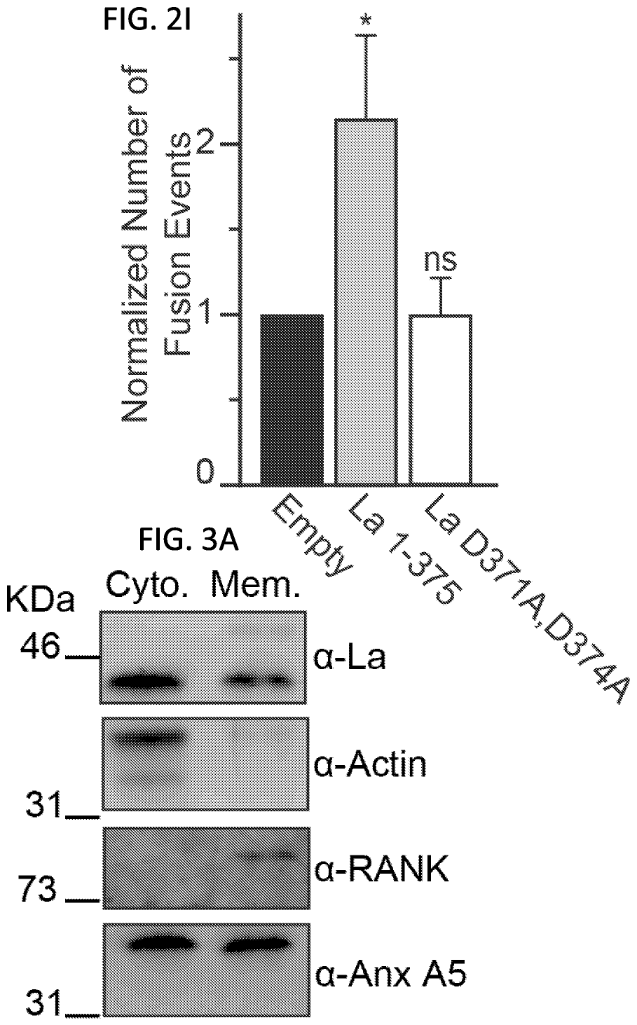

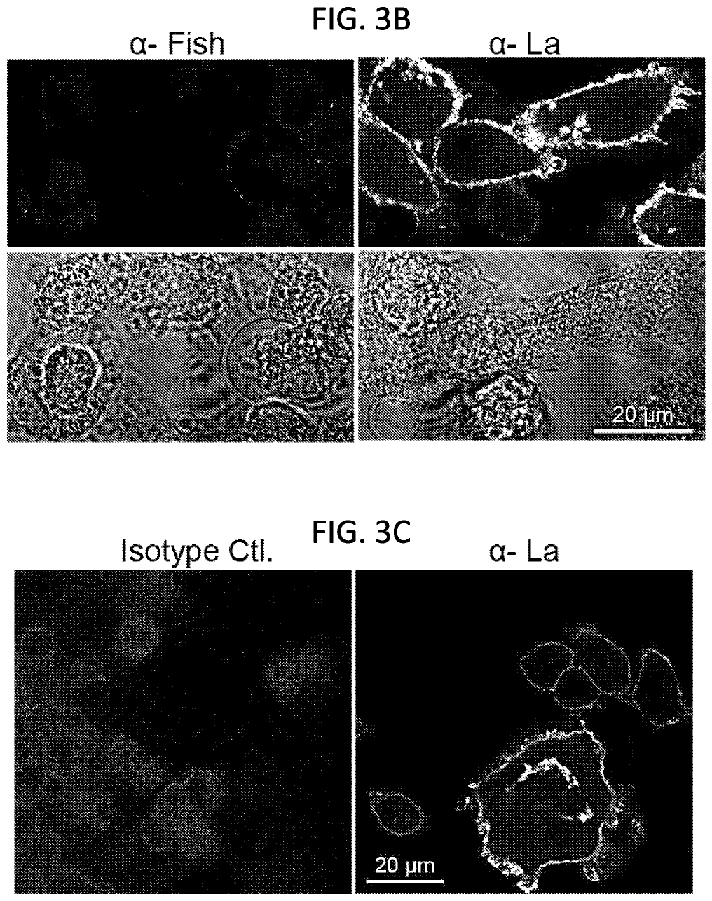

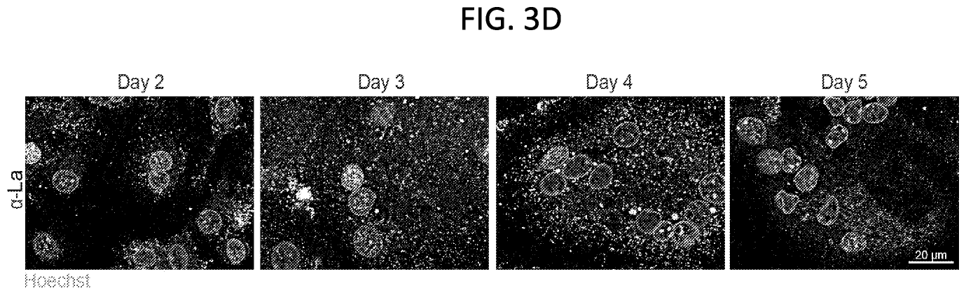

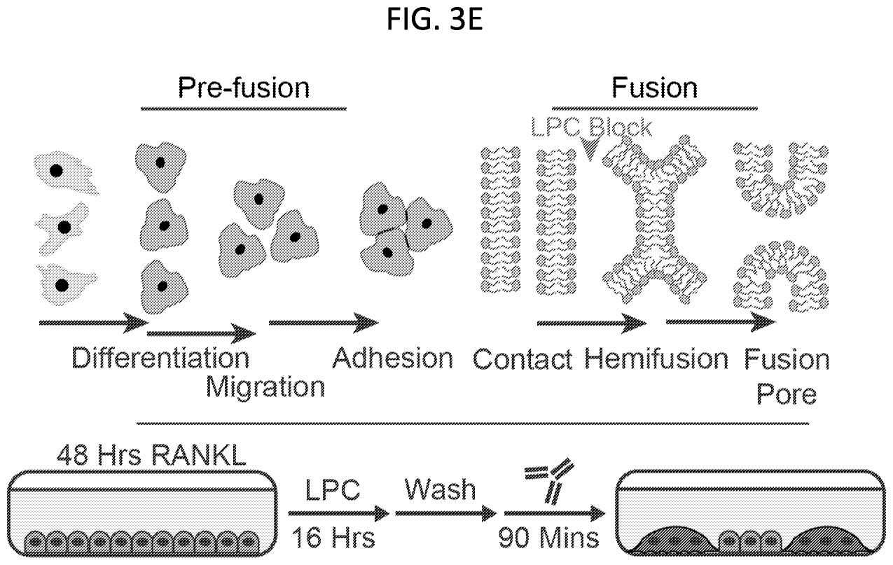

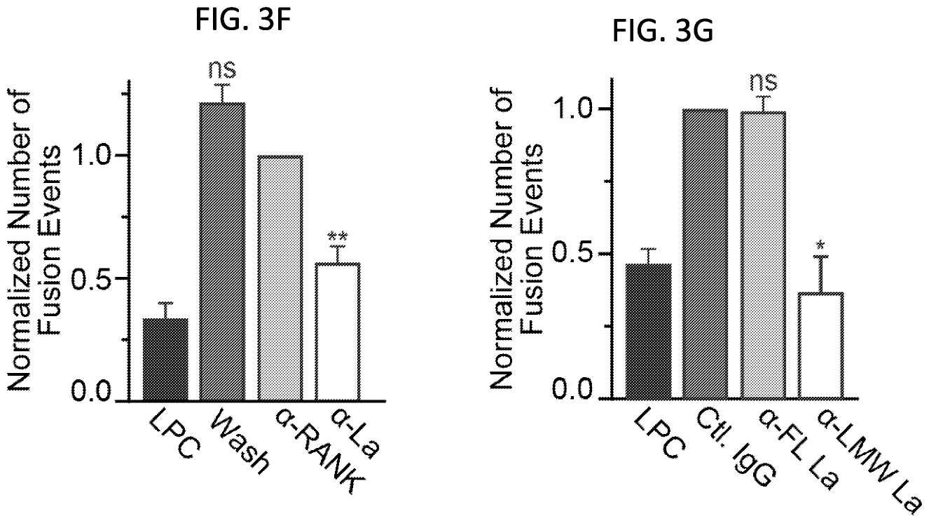

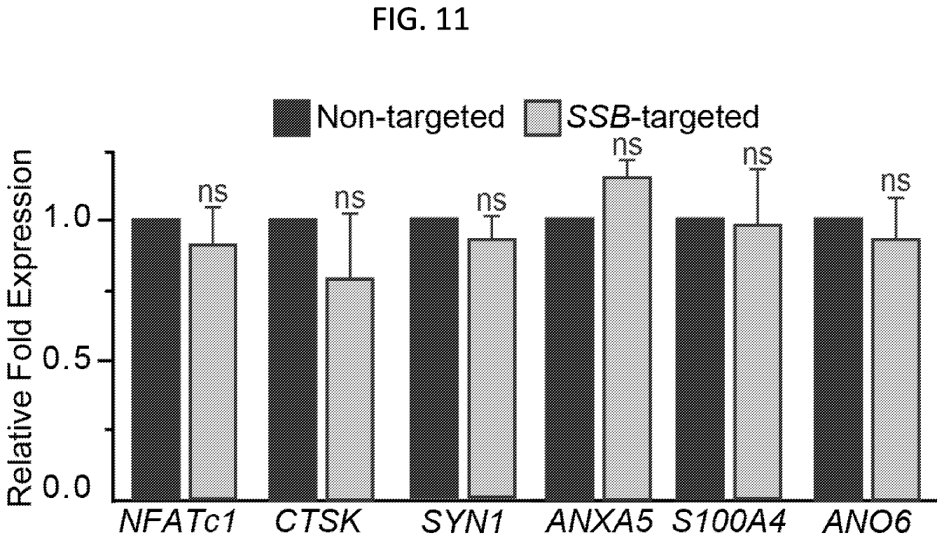

In the characterization of La's role in osteoclastogenesis, it was first explored whether La exerted its function in the formation of osteoclasts indirectly by altering the expression of factors implicated in osteoclastogenic differentiation or osteoclast fusion. While La expression in many cell types influences the steady-state levels of many transcripts/proteins (Sommer et al., Oncogene 30, 434-444, doi:10.1038/onc.2010.425 (2011)), RNAi suppression of La did not alter the steady-state transcript levels of the essential osteoclastogenesis factors NFATc1 and CTSK or transcripts coding for the fusion associated proteins syncytin 1, Anx A5, S100A4 or the lipid scramblase anoctamin 6/TMEM16F (FIG. 11) (Verma et al., J Biol Chem 293, 254-270, doi:10.1074/jbc.M117.809681 (2018)). Therefore, while La knockdown inhibited the formation of osteoclast syncytia, it did not grossly impact osteoclast differentiation or other machinery critical for cell-cell fusion. To further explore the mechanism by which La influenced the formation of osteoclasts but not their differentiation, it was assessed whether the formation of multinucleated osteoclasts depends on La's well characterized RNA binding function. This highly conserved function is based on high affinity interactions between the La domain and its high affinity oligo(U)-3′ binding site common to RNA polymerase III transcripts. To assess the requirement of high affinity interactions between the La domain and transcripts in osteoclastogenesis, a mutant La 1-375 with three-point mutations known to functionally impair La domain function, Q20A/Y24A/D33I (Bayfield et al., Nat Struct Mol Biol 16, 430-437, doi:10.1038/nsmb.1573 (2009); Vinayak et al., Nucleic Acids Res 46, 4228-4240, doi:10.1093/nar/gky090 (2018)). (La 1-375 RNAΔ) was overexpressed. It was found that La 1-375 RNAΔ promoted formation of multinucleated osteoclasts as robustly as wild type La 1-375, indicating that the La domain's high-affinity for RNA polymerase III transcripts is dispensable for La's role in osteoclast formation (FIGS. 2F, 3G).

The second piece of evidence suggesting that La's role in the formation of multinucleated osteoclasts was not through La's canonical role in RNA metabolism was the demonstration that La's function in osteoclasts was not in the nucleus or the cytoplasm, but rather, at the cell surface. As noted earlier, in differentiating osteoclasts La loses its NLS and appears in punctate structures throughout the cell. Proteins were enriched from RANKL committed osteoclasts at timepoints when cells were actively fusing into soluble, cytosolic or membrane-associated protein fractions. As expected, actin was found mostly in the cytosolic fraction, transmembrane RANK receptor in the membrane fraction, and the peripheral membrane protein Anx A5 in both fractions (FIG. 3A). While La protein is putatively considered a soluble protein, in differentiating osteoclasts, La was found in both cytosolic and membrane-associated fractions, suggesting La was unexpectedly associating with membranes during osteoclastogenesis (FIG. 3A).

In earlier reports, La cleavage in apoptotic cells was associated with the detection of La on the cell surface (Rutjes et al., Cell Death Differ 6, 976-986, doi:10.1038/sj.cdd.4400571 (1999); Ayukawa et al., J Biol Chem 275, 34465-34470, doi:10.1074/jbc.M003673200 (2000)), however whether this surface La played some cellular function or operated simply as a surface antigen was previously unknown. To assess whether osteoclast La traffics to the cell surface following cleavage, fusing osteoclasts were stained with α-La antibodies under non-permeabilizing conditions (FIG. 3B, 3C). In contrast to the osteoclast peripheral membrane protein Fish (also known as TSK5) (Oikawa et al., J Cell Biol 197, 553-568, doi:10.1083/jcb.201111116 (2012)), which is enriched during osteoclast fusion and binds to the cytoplasmic leaflet of the plasma membrane (PM) (Oikawa et al., J Cell Biol 197, 553-568, doi:10.1083/jcb.201111116 (2012)), La abundantly decorated the surface of fusing human osteoclasts (FIG. 3B). Moreover, this surface pool of La is not exclusive to human osteoclasts. Significant La surface staining was observed in RAW 264.7 derived, murine osteoclasts (FIG. 3C), suggesting surface La is a feature common to fusing osteoclasts in mammals.

Using surface staining with α-La at different days post RANKL application, the transient increase in La at the time points associated with robust fusion was observed (FIG. 3D vs. FIG. 1B), further implicating La in fusion.

It was then assessed whether La at the surface of human osteoclasts functions at the cell fusion stage of osteoclastogensis. All cell-cell fusion events in development and tissue maintenance proceed through slow (days), asynchronous differentiation processes that prepare fusion competent cells (Brukman et al., J Cell Biol 218, 1436-1451, doi:10.1083/jcb.201901017 (2019)). Then, PM fusion occurs by the rapid (minutes) progression from the initial formation of hemifusion connections to fusion pores that unite the volumes of two cells (FIG. 3D, top). These steps were decoupled in the formation of multinucleated syncytia using the hemifusion inhibitor lysophosphatidylcholine (LPC) (Verma et al., J Biol Chem 293, 254-270, doi:10.1074/jbc.M117.809681 (2018)). LPC's inverted cone shape is not conducive to the concave geometry of the hemifusion stalk, so ready-to-fuse cells are trapped upstream of hemifusion. After removing LPC, cells undergo synchronized fusion relatively rapidly (within 90 mins), affording the ability to assess the function of proteins specifically in the membrane fusion stage of osteoclast formation decoupled from upstream differentiation processes (FIG. 3D, bottom). Ready-to-fuse, RANKL committed cells were accumulated in the presence of LPC, and then this hemifusion blockade was lifted by washing out LPC. Application of α-La antibodies at the time of LPC removal significantly inhibited synchronized, osteoclast membrane fusion (FIG. 3E). In contrast, antibodies targeting the PM receptor RANK at the surface of osteoclasts had no effect (FIG. 3F). While RANKL-RANK signaling triggers upstream osteoclastogenic differentiation, inhibition of RANK following hemifusion synchronization fails to inhibit membrane fusion, as fusion itself does not depend on the activity of RANK (Verma et al., J Biol Chem 293, 254-270, doi:10.1074/jbc.M117.809681 (2018)). Moreover, while α-LMW La antibodies completely blocked synchronized osteoclast membrane fusion, α-FL La antibodies had no effect (FIG. 3G).

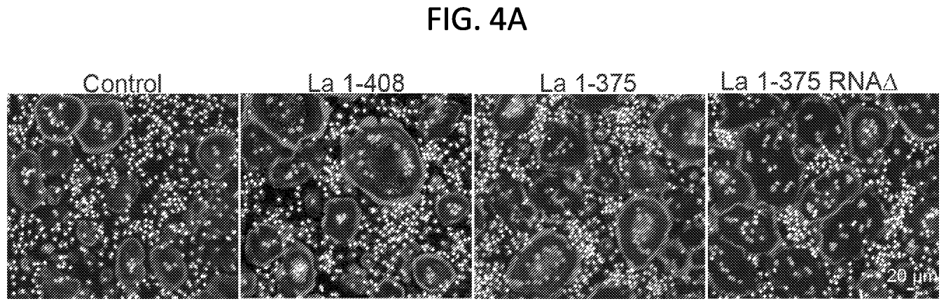

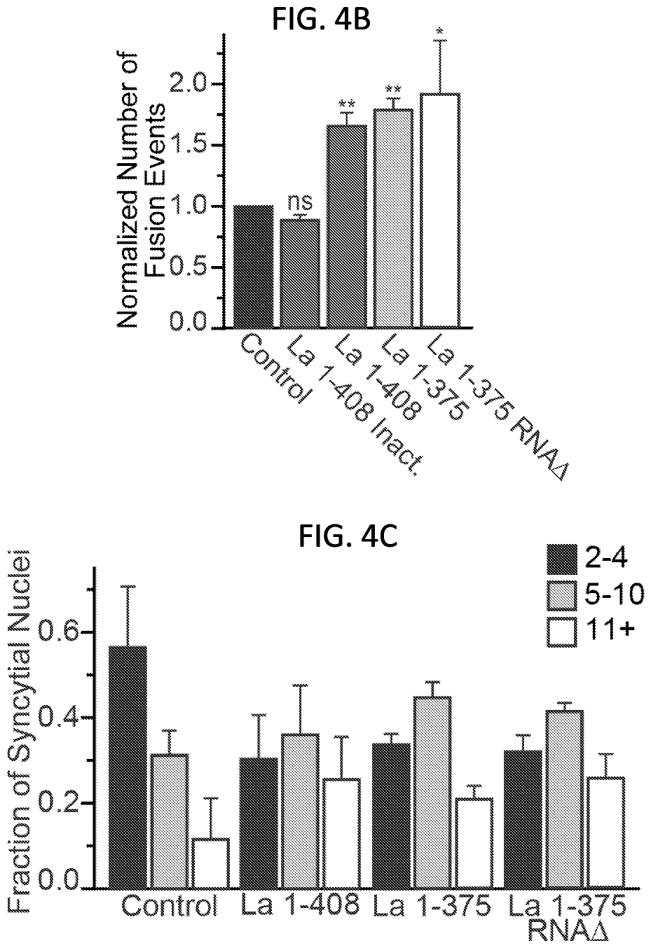

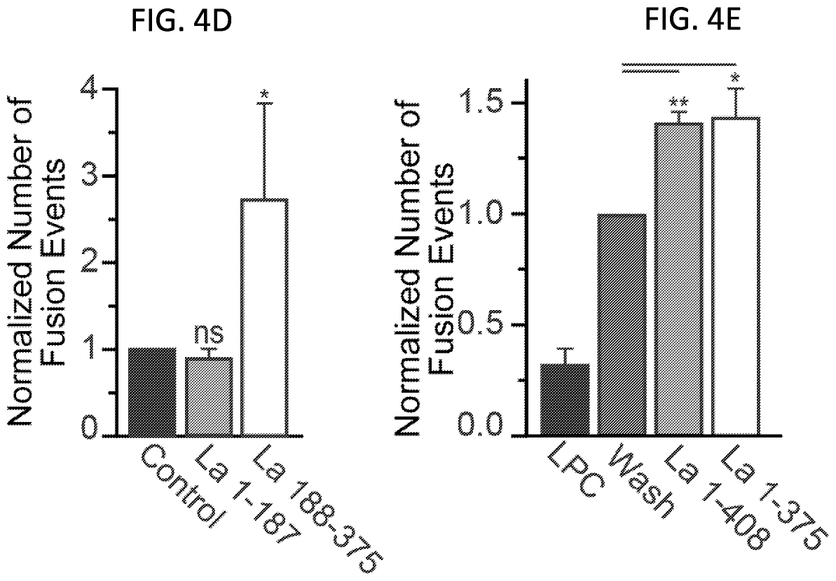

In contrast to the fusion-inhibiting effects of antibodies targeting surface La, application of recombinant La dramatically promoted osteoclast fusion. Application of FL La (La 1-408), truncated La (La 1-375) or truncated, RNA binding mutant La 1-375 RNAΔ outside fusing osteoclasts significantly promoted the formation of multinucleated syncytia (FIGS. 4A-4C). This promotion was not observed when recombinant La was heat inactivated (FIG. 4B). Recombinant La 1-375 RNAΔ promoted fusion similarly to La 1-408 and La 1-375, confirming that La's high-affinity interactions with RNA polymerase III transcripts are not required for La's role in regulating osteoclast fusion (FIGS. 4A-4C). Moreover, the ability of FL La to promote osteoclast fusion demonstrates that FL La itself is not fusion incompetent, but rather suggests that proteolytic processing and/or dephosphorylation of La are important for its delivery to the cell surface.

To further resolve the contributions of La domains critical for RNA binding, La and RRM1 domains (Wolin et al., Annu Rev Biochem 71, 375-403, doi:10.1146/annurev.biochem.71.090501.150003 (2002); Maraia et al., Wiley Interdiscip Rev RNA 8, doi:10.1002/wrna.1430 (2017); Bayfield et al., Nat Struct Mol Biol 16, 430-437, doi:10.1038/nsmb.1573 (2009); Vinayak et al., Nucleic Acids Res 46, 4228-4240, doi:10.1093/nar/gky090 (2018)), La 1-375 was split into La 1-187 and La 188-375. It was found that La 188-375 greatly promoted the formation of multinucleated osteoclasts, whereas La 1-187 had no effect (FIG. 4D). These data demonstrate that the La domain, RRM1 and La's C-terminal 33 AAs are dispensable for La's role in osteoclast formation (FIG. 4D). Importantly, La promoted the formation of osteoclasts at the membrane fusion stage rather than some pre-fusion stage of the differentiation. To this point, application of recombinant La to LPC-synchronized osteoclasts dramatically promoted osteoclast membrane fusion (FIG. 4E).

All these data indicated that La functions at the cell surface during the membrane fusion stage of osteoclast formation to promote the formation of large, multinucleated osteoclasts. Proteins involved in membrane fusion can be divided into protein fusogens that are sufficient for generating hemifusion intermediates and opening of fusion pores, and proteins that regulate fusogen activity (Bruckman et al., J Cell Biol 218, 1436-1451, doi:10.1083/jcb.201901017 (2019)). To test whether cell surface La may fuse membranes on its own, functioning as an active protein fusogen, La's ability was assessed to promote fusion between 3T3 fibroblasts, stably expressing HA0 (an uncleaved form of the influenza fusogen hemagglutinin (HA)), and red blood cells (RBCs) labeled with lipid and a content probes (Leikina et al., Dev Cell 46, 767-780 e767, doi:10.1016/j.devce1.2018.08.006 (2018)). HA0 is fusion-incompetent but establishes very tight contacts between HA0-expressing fibroblasts and RBCs. It was found that none of 872 analyzed HA0-cell bound RBCs exchanged lipid (hemifusion indicator) or cytoplasmic (fusion pore indicator) probes in response to application of 40 nM recombinant La. Based on Wilson's method (Ludbrook et al., ANZ J Surg 79, 565-570, doi:10.1111/j.1445-2197.2009.04994.x (2009)), the probability of La mediated fibroblast-RBC fusion does not exceed 0.0044 per cell contact and, under these conditions, exhibits at least 100-fold lower activity than the fusion mediated by a bonafide fusogen-activated HA (˜0.5 per contact). These data indicate that La does not itself have detectable cell-cell fusogenic activity, but rather, La likely regulates some larger fusion mechanism specific to osteoclasts.

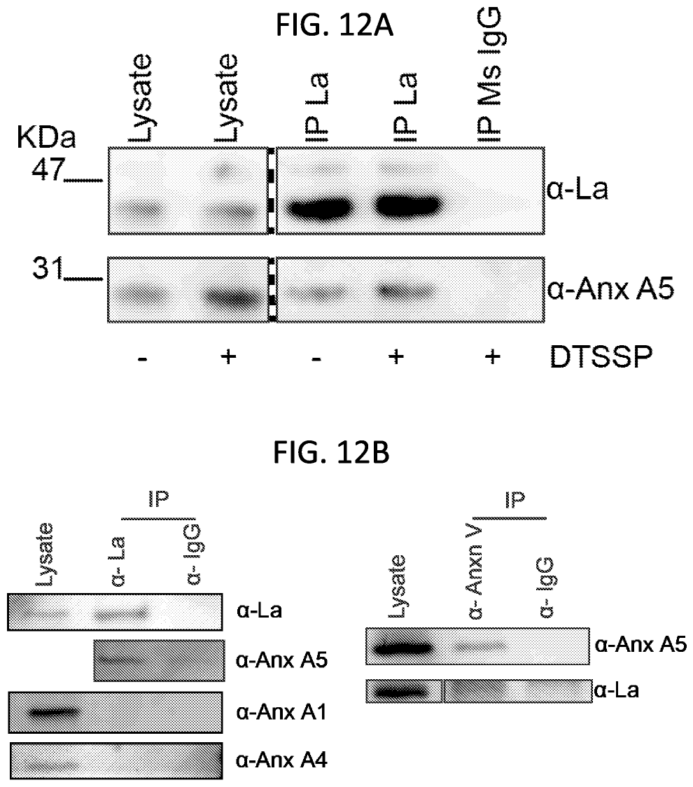

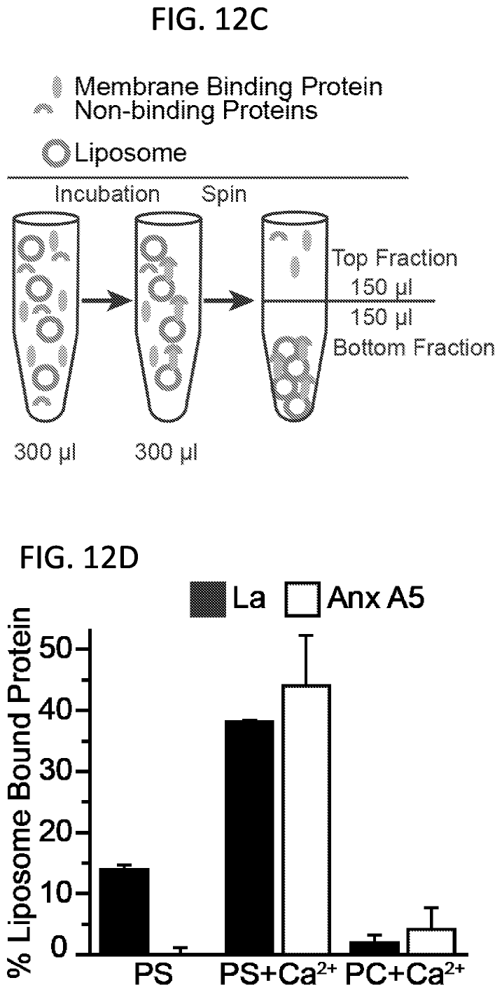

The lack of a direct fusogenic activity of La suggested that cell surface La interacts with some other protein(s) involved in fusion. To test this hypothesis, it was assessed whether La interacted with Anx A5, a peripheral membrane protein, also involved in membrane fusion stage of osteoclast formation (Verma et al., J Biol Chem 293, 254-270, doi:10.1074/jbc.M117.809681 (2018); Whitlock and Chernomordik, J Biol Chem, 100411, doi:10.1016/j.jbc.2021.100411 (2021)) and upregulated at similar timepoints in osteoclastogenesis. La and La-containing protein complexes were immunoprecipitated from fusing human osteoclasts on magnetic beads with α-La Abs and it was found that La protein complexes contained Anx A5 (FIG. 12A). La supramolecular complexes from fusing osteoclasts contained neither Anx A1 nor Anx A4, both abundant in fusing osteoclasts (FIG. 12B), demonstrating specificity in La's association with Anx A5. Moreover, immunoprecipitations of Anx A5 supramolecular complexes from fusing human osteoclasts isolated using α-Anx A5 Abs, contain La (FIG. 12B).

Additional evidence for La-Anx A5 interactions came from the experiments in which it was found that direct binding of La to Anx A5 anchors La on phosphatidylserine (PS) containing phospholipid liposomes. Anx A5 binds to PS containing membranes in a Ca²⁺-dependent manner. Recombinant La was introduced alone or along with recombinant Anx A5 to PS containing liposomes and pelleted liposomes by centrifugation to evaluate whether La was enriched with liposomes or in supernatant (FIG. 12C). La alone pelleted poorly with liposomes, as it does not have membrane binding domains itself. In contrast, both La and Anx A5 pelleted with liposomes in response to Ca²⁺ (FIG. 12D). La membrane association required Anx A5, Ca²⁺ and PS, as neither La nor Anx A5 pelleted with liposomes lacking PS (FIG. 12D). These findings suggest that direct interactions between La and extracellular Anx A5 bound to PS, transiently exposed at the surface of osteoclasts at the time of fusion (Verma et al., J Biol Chem 293, 254-270, doi:10.1074/jbc.M117.809681 (2018)), facilitate La association with the surface of differentiating osteoclast precursors.

### Example 3

### La Presents a Potential Target for Influencing Osteoclast Formation and Function

It was demonstrated that cell-surface La regulates formation of human and murine multinucleated osteoclasts triggered by biologically relevant interactions between osteoclast precursors and bone-forming cells. Targeting La modulates fusion between osteoclast precursors and, in turn, alters the propensity of the resulting osteoclasts to resorb bone. Furthermore, it was found that La is involved in osteoclast formation in an ex vivo FD model evidence that targeting La function at the surface of developing osteoclasts is an effective therapeutic intervention in FD, and other resorptive bone diseases stemming from excessive osteoclast activity.

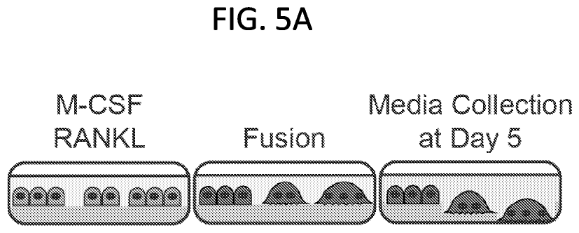

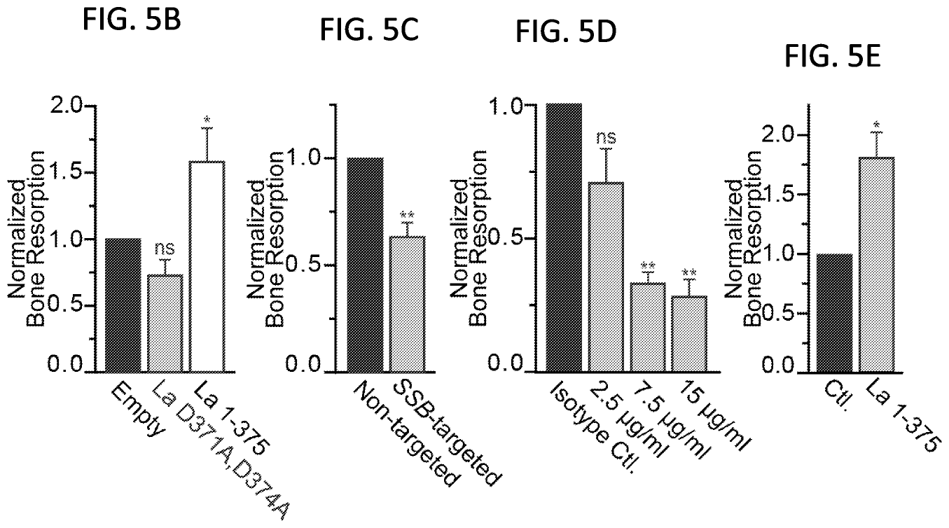

There is a relationship between osteoclast fusion and bone resorption (Makris and Saffar, Arch Oral Biol 27, 965-969, doi:10.1016/0003-9969(82)90104-2 (1982); Piper et al., Anat Embryol (Berl) 186, 291-299, doi:10.1007/BF00185977 (1992); Moller et al., Int J Mol Sci 21, doi:10.3390/ijms21176368 (2020)). It was hypothesized that by regulating osteoclast size, La also regulates bone resorption. This hypothesis was evaluated by differentiating osteoclasts on fluoresceinated calcium phosphate, a biomimetic of bone, and assessed osteoclast-dependent bone resorption by the release of fluorescein into the media (FIG. 5A). Monocyte derived precursors (only M-CSF) released minimal trapped fluorescein, but the addition of RANKL resulted in formation of multinucleated osteoclasts that readily resorbed calcium phosphate and released fluorescein (FIG. 8B). Overexpression of La 1-375 promoted bone resorption, while the uncleavable La mutant, D371,374A, had no effect (FIG. 5B). Moreover, RNAi mediated reduction of La reduced bone resorption by ˜40% compared to non-targeted controls (FIG. 5C). α-La antibodies that inhibit fusion (FIG. 3E) also dramatically reduced osteoclast-dependent bone resorption in a dose dependent manner (FIG. 5D). Finally, the extracellular addition of recombinant La 1-375 to fusing osteoclasts dramatically increased osteoclast bone resorption (FIG. 5E). Based on these data, it was concluded that targeting cell surface La bidirectionally regulates both osteoclast fusion and subsequent bone resorption.

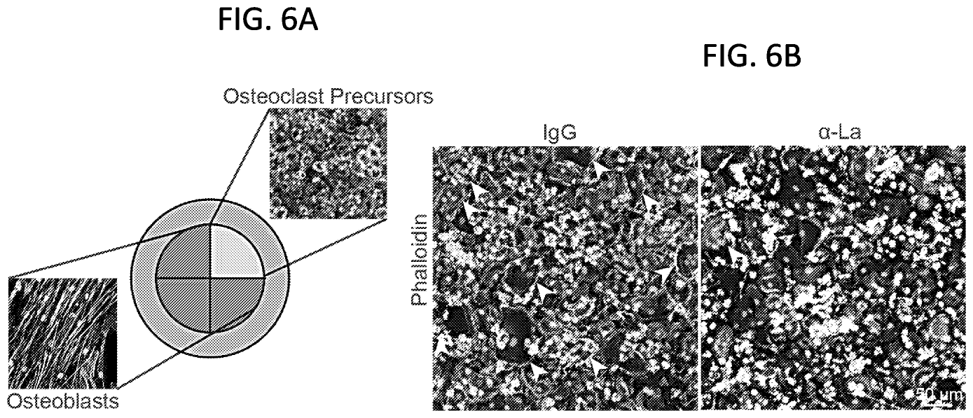

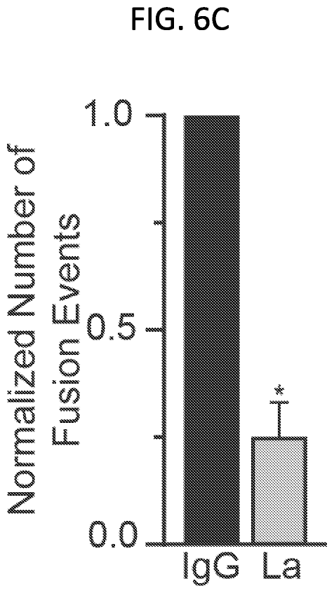

In biologically relevant situations, osteoclastogenesis develops in the context of interactions between osteoclast precursors with bone-forming osteoblasts/osteocytes and other cell types, generating much lower concentrations of RANKL and many other osteoclastogenesis-regulating factors (Kitura et al., Int J Mol Sci 21, doi:10.3390/ijms21145169 (2020)). To explore whether La is involved in osteoblast-induced osteoclast formation, primary human osteoblasts isolated from trabecular bone were co-cultured with human osteoclast precursors, derived via M-CSF induction of primary human monocytes. Osteoblasts and osteoclast precursors were cultured isolated from each other by well inserts (FIG. 6A). Without removing well inserts, no fusion was observed between osteoclast precursors. Upon removal of well inserts, media from the osteoblast/osteoclast wells mixed and co-cultured osteoclast precursors rapidly fused to produce multinucleated osteoclasts. Addition of α-La antibody blocked nearly 75% of the fusion between osteoclasts in such co-cultures (FIGS. 6B, 6C) confirming the involvement of La in osteoclast formation in a biologically relevant model of bone remodeling lesions.

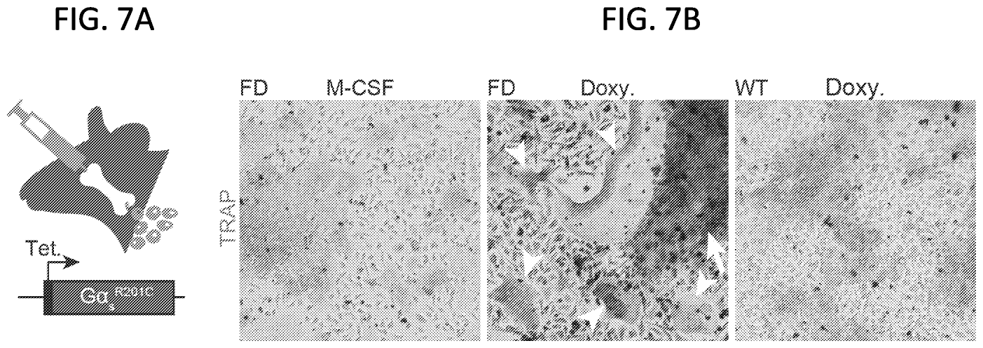

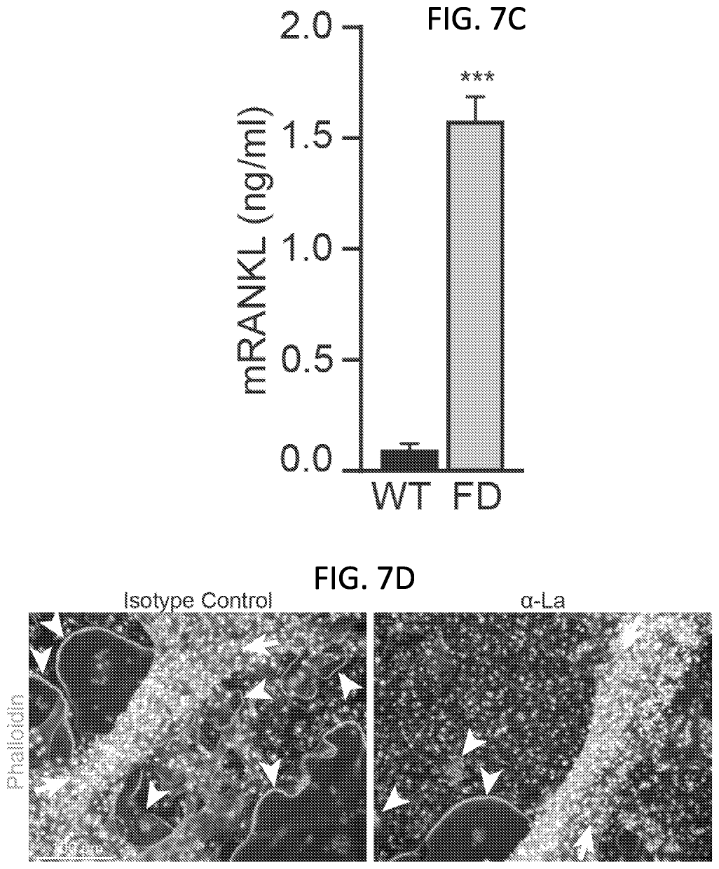

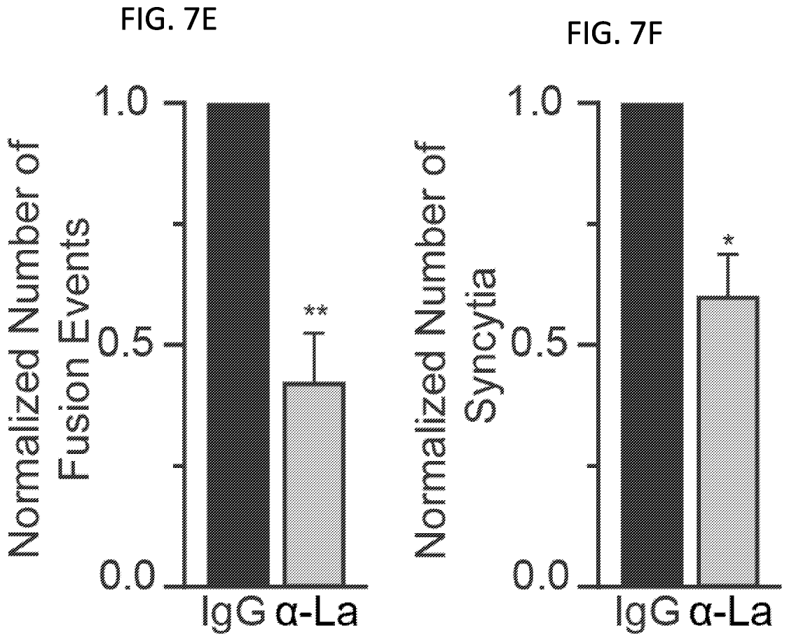

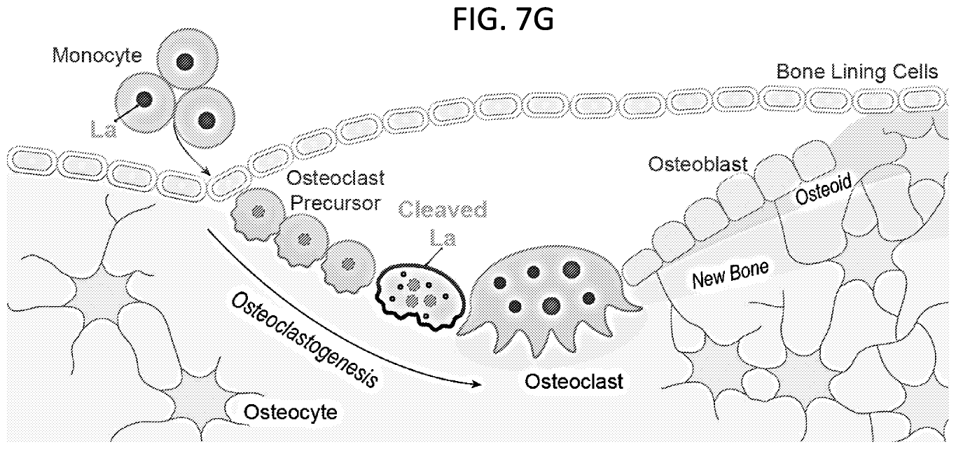

To explore whether La function plays a role in bone pathology, experiments were focused on fibrous dysplasia of bone (FD), an osteoclast-dependent bone disease (de Castro et al., J Bone Miner Res 34, 290-294, doi:10.1002/jbmr.3602 (2019)). FD is caused by gain-of-function mutations in Gas that lead to constitutively increased cAMP signaling and upregulation of cAMP/RANKL-dependent osteoclastogenesis (Boyce and Collins, Endocr Rev 41, doi:10.1210/endrev/bnz011 (2020)). In a conditional, tetracycline inducible mouse model, FD-like bone lesions, develop in adult mice within 2 weeks following doxycycline (Doxy) administration (Xao et al., Proc Natl Acad Sci USA 115, E428-E437, doi:10.1073/pnas.1713710115 (2018)). The formation of these lesions is driven by activation of an inducible gain-of-function mutant, Gαₛ^{R201C}, specifically in cells of the skeletal stem cell linage responsible for the excessive RANKL production observed in FD. This excessive RANKL production results in the ectopic formation of numerous, large osteoclasts that excessively erode healthy bone. Using bone marrow explants from these FD mice, a robust ex vivo model was established of the ectopic osteoclast formation observed in FD (FIG. 7A). As depicted in FIG. 7B, culture of these FD explants in the presence of M-CSF alone resulted in numerous adherent cells but no multinucleated, TRAP⁺ osteoclasts. In contrast, addition of Doxy resulted in the rapid development of fibrous cell clumps (arrow) and numerous multinucleated, TRAP⁺ osteoclasts (arrowheads) that were not observed in explants from wild-type littermates, lacking the inducible Gαₛ^{R201C} element. Doxy-induced osteoclastogenesis was accompanied by a ˜17-fold increase in mRANKL produced by the explants (FIG. 7C). Importantly, α-La antibodies blocked osteoclast fusion elicited by the addition of Doxy to FD explants by ˜60% and reduced the number of multinucleated osteoclasts observed by ˜40% (FIGS. 7D-7F).

The differentiation of murine and human monocytes into multinucleated osteoclasts is dependent on tightly choreographed changes in the steady-state level, post-translational modification and cellular localization of La (FIG. 7G). At the onset of osteoclastogenesis, M-CSF-derived precursors show a dramatic loss of La protein, suggesting that this differentiation process may require the concerted downregulation of a specific La-regulated pool of mRNAs triggered by the loss of steady state La. In the following RANKL-induced stages of osteoclastogenesis, La reappears as a non-phosphorylated, proteolytically cleaved species in the cytoplasm and at the surface of the fusing osteoclast precursors. When the growth of osteoclasts slows, in the late stages of fusion, La is observed at its conventional size and nuclear localization. The rate of formation, the sizes of multinucleated syncytia and the subsequent bone resorption activity of osteoclasts are regulated by cell-surface La protein. In fact, cell-surface La regulates osteoclast functions by modulating the membrane fusion stage of osteoclast formation, not upstream differentiation processes. Lowering the amount of La by suppressing the steady-state level of its transcript, blocking the proteolytic processing required for its trafficking to the surface of the cells, or inhibiting its activity with antibodies inhibits fusion. Conversely, increasing La's steady-state concentration by either overexpression or application of recombinant protein promotes fusion. In summary, these data demonstrate that La, a key protein in the RNA biology of eukaryotic cells, is present at the surface of osteoclasts where it is a master regulator of osteoclast membrane fusion.

The data presented herein demonstrate that La's role in regulating osteoclast fusion and bone resorption is separate from the well-described canonical functions of La, and represents a novel function for the La protein. First, the ability to inhibit or promote synchronized osteoclast membrane fusion by non-membrane permeable reagents (e.g., antibodies, recombinant La) indicates that the regulation of osteoclast fusion depends on surface La.

La regulation of osteoclast formation does not depend on interactions between the highly conserved La domain and RRM1 of La with RNA. This conclusion is supported by the finding that neither mutations in critical residues within the La domain nor deleting the entire N-terminal half of La protein (containing both the La domain and RRM1) abolishes the ability of the recombinant La to promote osteoclast fusion.

Since La, on its own, initiates neither hemifusion nor fusion between bound membranes, it is unlikely that La directly catalyzes and/or drives membrane fusion. More likely La recruits or stimulates other components of the osteoclast fusion complex. The latter scenario is supported by the present findings that highlight La's association with the fusion regulator Anx A5. It was found that recombinant La and Anx A5 directly interact, and that Anx A5 facilitates the association of La with membranes containing PS in a Ca2+-dependent manner. These observations in combination with the previously reported dependence of osteoclast fusion on cell surface PS and Anx A5 (Verma et al., J Biol Chem 293, 254-270, doi:10.1074/jbc.M117.809681 (2018)) shed light on how osteoclasts may employ PS to trigger the assembly of a fusion complex between committed precursors.

Imbalance of bone-formation and resorption in many skeletal diseases is linked to either excessive (e.g., osteoporosis, Paget's disease and FD), or insufficient activity of osteoclasts (e.g., osteopetrosis). It is disclosed herein that the formation of multinucleated, human osteoclasts can be inhibited or promoted by treatments targeting La protein at the surface of osteoclast precursors. Importantly, α-La antibodies inhibit fusion and bone resorption by osteoclasts derived from RANKL activated monocytes. α-La antibody treatment also inhibited the formation of multinucleated osteoclasts in human osteoclast precursor/osteoblast co-cultures modeling bone remodeling lesions, where osteoclastogenic factors are produced by osteoblasts within the lesion (Ikebuchi et al., Nature 561, 195-200, doi:10.1038/s41586-018-0482-7 (2018)). Moreover, the hypothesis that cell-surface La plays an important role in osteoclast formation within biologically relevant contexts was substantiated by the experiments in an ex vivo model of FD. Development of FD is characterized by drastically increased levels of RANKL and other osteoclastogenic factors in serum and the excessive, ectopic formation of numerous multinucleated osteoclasts in the vicinity of bone lesions. As expected, induction of the FD phenotype in bone marrow explants resulted in high concentrations of RANKL and ectopic osteoclastogenesis. Finding that α-La inhibits the formation of multinucleated osteoclasts, both in size and number, confirmed the importance of La as a novel target in bone pathologies and highlighted La as a target for future therapeutic development.

Some of the proteins involved in the early stages of osteoclastogenic differentiation have already been tested in animal and/or clinical studies as potential therapeutic targets (Boyce et al., J Bone Miner Res 28, 711-722, doi:10.1002/jbmr.1885 (2013)). The α-RANKL antibody denosumab is an FDA-approved drug for the treatment of osteoporosis (Sordillo et al., Cancer 97, 802-812, doi:10.1002/cncr.11134 (2003)). La-dependent osteoclast fusion, downstream of the RANKL/RANK/osteoprotegerin signaling pathway, presents a target for therapies at a different mechanistic stage of bone remodeling. Taking into account that mononucleated osteoclasts do resorb bones, blocking La-dependent osteoclast fusion can have more subtle and selective effects on bone resorption than blocking the upstream formation of osteoclast precursors with α-RANKL antibodies (see, for example, Miyamoto et al., J Bone Miner Res 27, 1289-1297, doi:10.1002/jbmr.1575 (2012)). Like RANKL, cell surface La is accessible for cell-impermeable drugs. In some clinical situations, more subtle action of La-targeting treatments can be advantageous. Furthermore, unlike RANKL, which in addition to osteoclastogenesis regulates immune response (Ono et al., Inflamm Regen 40, 2, doi:10.1186/s41232-019-0111-3 (2020)), the only known function of cell surface La is its disclosed role in regulating osteoclast fusion. Thus, surface La's specificity may minimize off-target effects. Finally, osteoclasts are known to release factors that regulate osteoblast activity (Sims et al., Curr Osteoporos Rep 10, 109-117, doi:10.1007/s11914-012-0096-1 (2012)). Blocking osteoclastogenesis altogether by targeting RANKL likely blocks osteoclast-osteoblast signaling. Suppressing the fusion stage of osteoclast formation by targeting La, while maintaining the ability of osteoclast to differentiate, can maintain this osteoclast-osteoblast crosstalk within the bone remodeling lesion, which can be important for rebuilding bone in disease where it has been lost. In summary, the disclosed data demonstrate a function of La protein as a key regulator of osteoclast formation, a role strikingly different in place of action, mechanism, and partner protein (Anx A5) from its well-recognized functions as an RNA-chaperone, and provide therapeutic treatments for that modulate osteoclast function.

### Example 4

### Use of La Antibody in a Model of Fibrous Dysplasia (FD)

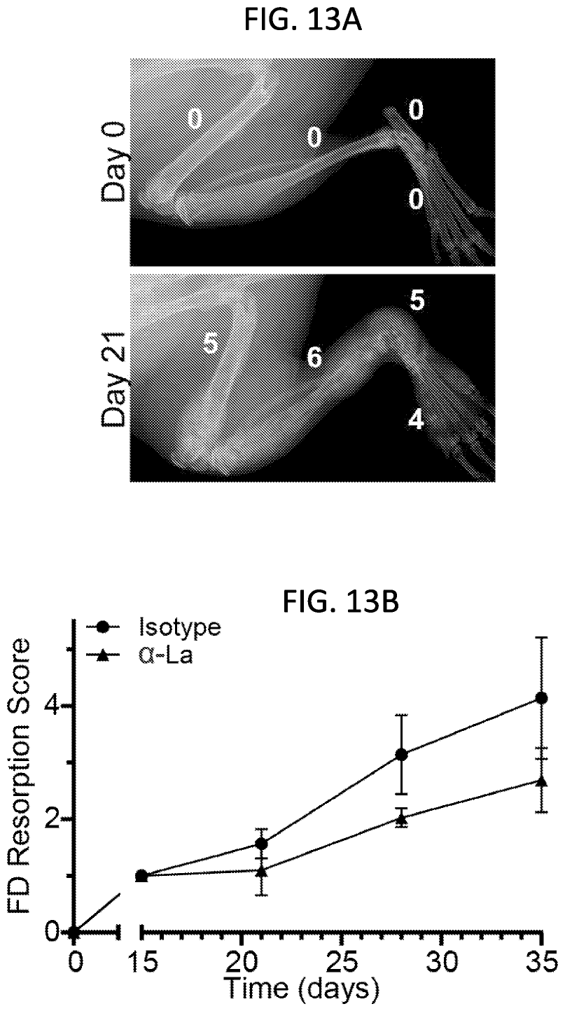

FD is an osteolytic bone disease in which an imbalance in osteoclast-osteoblast (OC-OB) signaling and coordination is central to the pathophysiology. Postzygotic, mosaic Gas mutations only in osteoblasts perturb OB-to-OC signaling, causing overproduction of osteoclastogenic signals, and alter osteoclast formation and activity. An inducible mouse model of FD was used, where addition of doxycytline to the foodstuff induces FD progression in ˜3 weeks. FD progression was detected by visually observing the loss of bone in the hindlimbs of the mice via x-ray timecourse, and the progression of FD was scored by observing the development of radiotranslucent resorption pits along the minerlized bones and the deposition of woven bone. Each bone was given a score from 1-6 denoting the extent of bone loss and average these scores to give each mouse an FD progression score (FIG. 13A). It was found that, in contrast to isotype control treated animals, α-La antibody administration suppresses the continued development of FD in the mouse model (FIG. 13B).

### Example 5

### Inhibitory Domains of La Protein

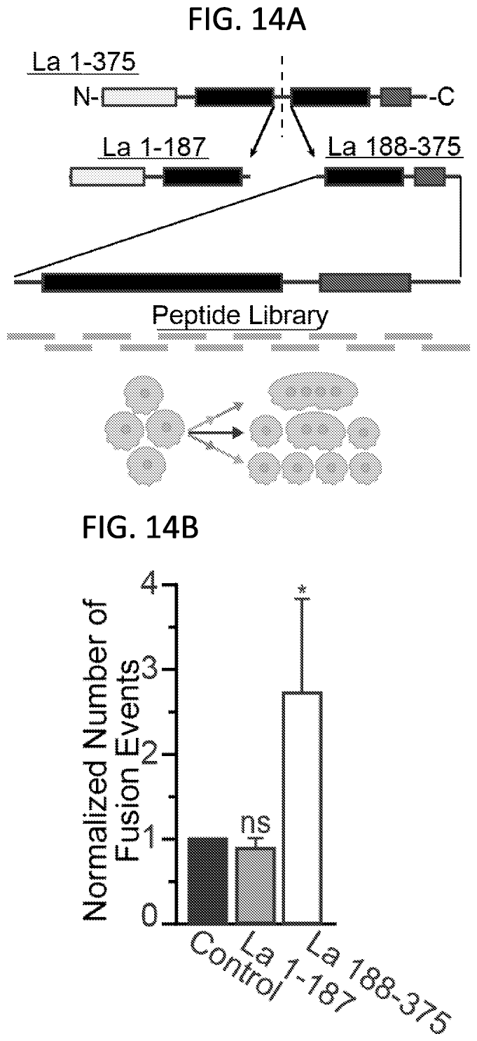

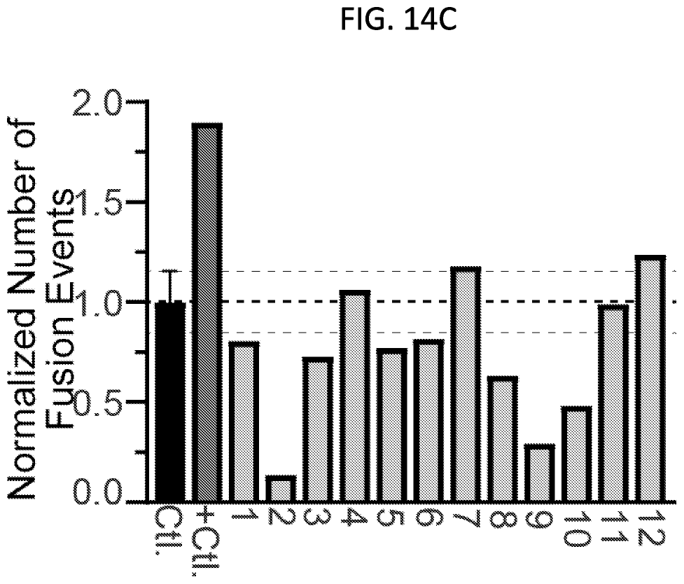

La is a canonical nuclear regulator of RNA metabolism in eukaryotes. A second function was detected, where La is cleaved, exposed on the PM, and controls osteoclast multinucleation and activity. The first 187 AAs of La (e.g., aa 1-187 of SEQ ID NO: 2), containing domains essential for canonical La-RNA binding, are dispensable for La's ability to promote osteoclast multinucleation (FIG. 14B). It was hypothesized that the uncharacterized region between AAs 188-375 contains La domain(s) that regulate osteoclast multinucleation. To identify the domain(s) within AAs 188-375 responsible for regulating osteoclast multinucleation, a peptide library was synthesized, consisting of overlapping peptides (30 AAs in length (12 total) (FIG. 14A)) spanning AAs 188-375. By screening the peptides, two peptides (2, SEQ ID NO: 8, and 9, SEQ ID NO: 9) were identified that specifically inhibit osteoclast multinucleation (FIG. 14C). In this figure, the control is no addition, and the + is the addition of LA 188-375, which promoted fusion.

Peptide 2:
(SEQ ID NO: 8)
AKLRAKQEQEAKQKLEEDAEMKSLEEKIGC;
or
Peptide 9:
(SEQ ID NO: 9)
GEVEKEALKKIIEDQQESLNKWKSKGRRFK

### Example 6

### Interaction of La and Annexin A5 (Anx A5)

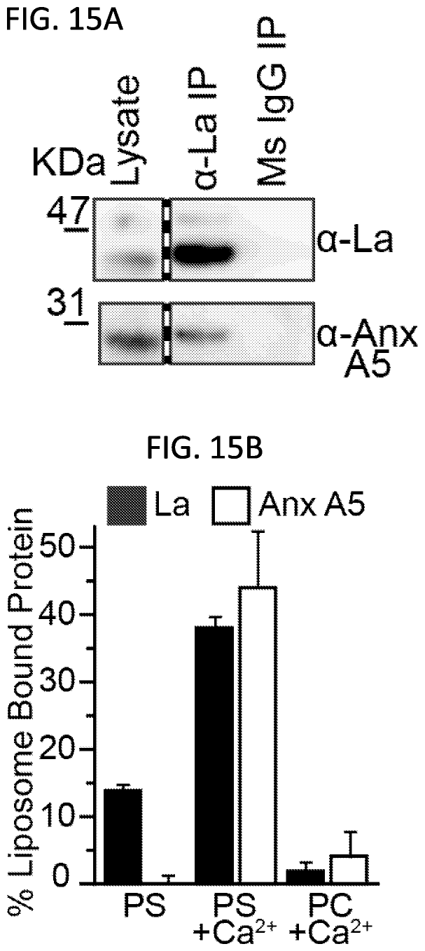

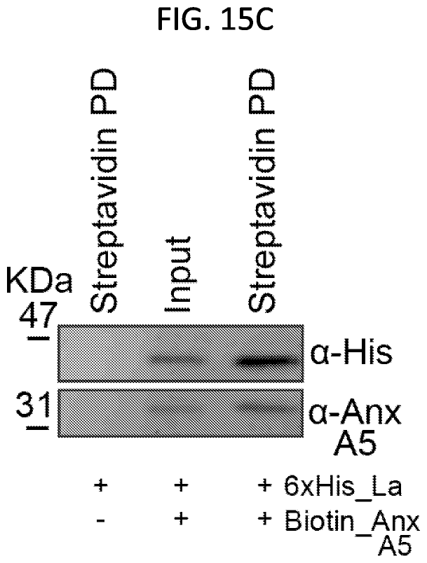

Anx A5 associates with the surface of fusing osteoclasts by binding phosphatidylserine (PS) in a Ca2+-dependent manner. Transient, plasma membrane (PM) PS exposure and Anx A5 binding promote osteoclast fusion. Data demonstrated that native La interacts with native Anx A5 in fusing osteoclasts, and that recombinant Anx A5 enriches recombinant La on membranes in a PS- and Ca2+-dependent manner (FIG. 15A, 15B). This indicates that Anx A5 enriches La at osteoclast PMs transiently exposing PS at the time and place of osteoclast fusion. It was hypothesized that direct La-Anx A5 protein interactions promote osteoclast multinucleation by enriching La at the surface of fusing osteoclasts. An assay was developed for evaluating direct La-Anx A5 binding by incubating recombinant 6×his-La and Biotin-Anx A5 in solution, pulling down Biotin-Anx A5, and evaluating 6×his-La co-precipitation (FIG. 15C). The La peptide library described in Example 5 is used to identify peptide(s) that reduce La co-precipitation and further evaluate La-Anx A5 binding in osteoclast formation.

### Example 7

### Treatment of Osteopetrosis by Suppressing Osteoclast Fusion

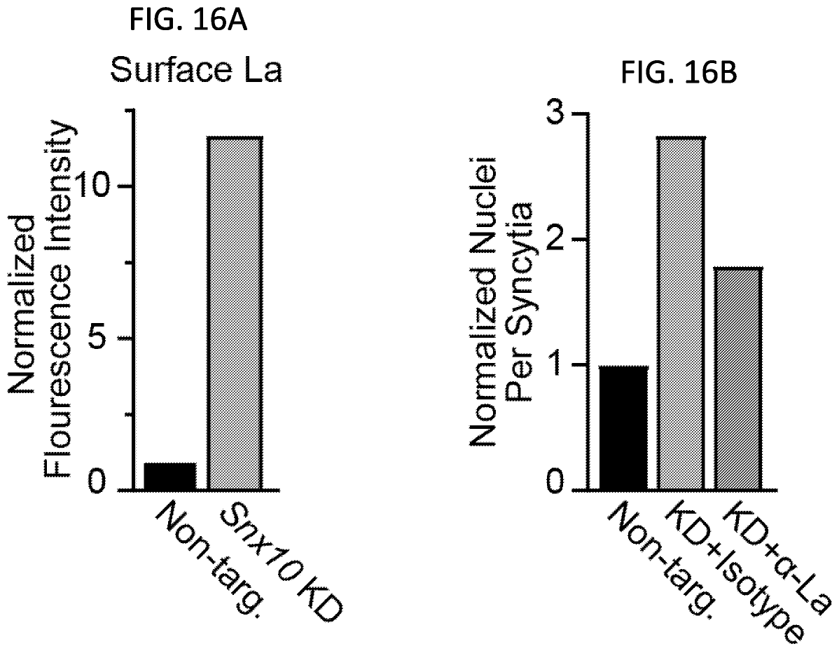

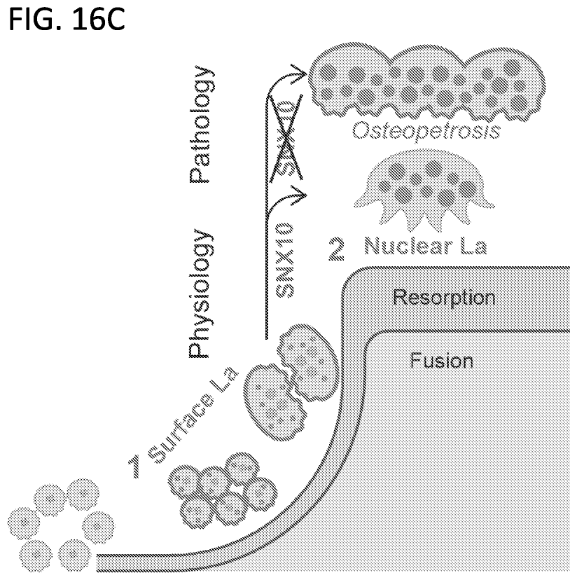

Changes in osteoclast size alter bone resorption and underpin bone diseases like Paget's disease and osteopetrosis. Loss of Snx10 produces extremely large OCs that no longer adhere to bone properly, exhibit defective resorption, and leads to osteopetrosis (M. Barnea-Zoha et al., 2021. An SNX10-dependent mechanism downregulates fusion between mature osteoclasts. J Cell Sci. 134(9): jcs254979. PMCID: PMC8182410; PMID: 33975343). The data indicated that loss of Snx10 greatly enriched La on the surface of OCs (FIG. 16A). Inhibiting this excessive surface La partially rescued the dysregulated osteoclast multinucleation observed following loss of Snx10 (FIG. 16B). The data indicate that SNX10 plays a role in degrading surface La and stopping osteoclast formation so that osteoclasts can resorb bone in a physiologically required manner. In this model, the loss of SNX10, which leads to osteopetrosis, causes defective osteoclast formation/function because it leaves La on the surface of osteoclasts, leaving them in a constant state of growing and resulting in defective attachment and function. By inhibiting surface La, defective OC formation can be rescued, and proper osteoclast function can be restored in osteoclast-rich osteopetrosis.

### Example 8

### Materials and Methods

Reagents: Human M-CSF and RANKL and murine M-CSF and RANKL were purchased from Cell Sciences (catalogue #CRM146B; #CRR100B; #CRM735B and CRR101D, respectively). LPC (1-lauroyl-2-hydroxy-sn-glycero-3-phosphocholine, #855475); PC (1,2-dioleoyl-sn-glycero-3-phosphocholine, #850375C); PS (1,2-dioleoyl-sn-glycero-3-phospho-L-serine, #840035C); lissamine rhodamine phosphatidylethanolamine (1,2-dioleoyl-sn-glycero-3-phosphoethanolamine-N-(lissamine rhodamine B sulfonyl, #810150C) were purchased from Avanti Polar Lipids. Bone Resorption Assay Kits were purchased from Cosmo Bio Co. (Catalogue #CSR-BRA-24KIT) and used according to the manufacturer's instructions. Hoechst 33342 and phalloidin-Alexa555 were purchased from Invitrogen (#H3570 and A30106, respectively). TRAP staining reagents were purchased from Cosmo Bio Co. (#PMC-AK04F-COS). The fluorescent lipid PKH26 (PKH26GL-1KT) and carboxyfluorescein, CF (5-(and-6)-Carboxyfluorescein, mixed isomers, #C368) were purchased from Sigma and Invitrogen, respectively.

Animals: A mouse model of fibrous dysplasia with inducible expression of hyperactive Gαₛ^{R201C} in cells of the osteogenic lineage (Zhao, X. et al., Proc Natl Acad Sci USA 115, E428-E437, doi:10.1073/pnas.1713710115 (2018); Boyce et al., Endocr Rev 41, doi:10.1210/endrev/bnz011 (2020)) was used to obtain bone marrow explants (described below). For this study 12-18 week old females were used.

Murine Bone Marrow Explant Culture: The tibia and femur were dissected from an inducible murine model of fibrous dysplasia described previously (Boyce et al., Endocr Rev 41, doi:10.1210/endrev/bnz0l 1 (2020)) or wild-type littermates. Holes were drilled into the epyphises of each bone using a 22-gauge hypodermic needle, and the bone marrow was flushed into a culture dish using alpha MEM. These bone marrow isolates were further dissociated through a fresh 22-gauge hypodermic needle to obtain a single cell suspension, and cultured in alpha MEM plus 20% FBS, 1×pen/strep and 1× Normocin (InvivoGen, #Ant-nr-1) for 7 days in T-75 culture flasks. Cells that adhered to the flask were washed 3 times with PBS and passaged using 0.05% Trypsin and a cell scraper and cultured for up to 3 passages in alpha MEM plus 20% FBS and 1×pen/strep. For Gαₛ^{R201C} expression induction in the bone marrow stromal cell subset of the explants, cells were plated at near confluency in 6-well plates and treated with 1 μM doxycycline (Sigma, #D9891-5G). During induction, media were refreshed daily. For antibody treatment, antibodies were added overnight when initial cell-cell fusion was observed (typically ˜4 days of doxycycline treatment) at 6 μg/ml overnight.

Cell Cultures:

Osteoclasts: Elutriated monocytes from healthy donors were obtained. Cells were plated at 2.9×10⁵ per cm² in 35 mm dishes with polymer coverslip bottoms (Ibidi #81156) for imaging or 35 mm or 10 cm dishes for biochemical experiments in complete media [α-MEM supplemented with 10% Fetal Bovine Serum (FBS) and penicillin-streptomycin-L-glutamine (Gibco Invitrogene #12571063; #26140079 and #10378016, respectively)]. Monocytes were differentiated to M2 macrophages in the presence of 100 ng/ml M-CSF for 6 days and then differentiated with 100 ng/ml M-CSF and 100 ng/ml RANKL for 3 days unless indicated otherwise. RAW 264.7 cells (ATCC, Manassas, VA, #TIB-71) were maintained in DMEM supplemented with 10% FBS to a maximum of 8 passages. RAW 264.7 cells were differentiated to osteoclasts in the presence of 100 ng/ml murine RANKL for 5 days. To separate unfused mononucleated and fused, multinucleated RAW 264.7 cells into separate fractions, advantage was taken of the much stronger adherence of multinucleated cells to culture dish plastic. After washing in PBS, mixed RAW cultures (following RANKL differentiation) were left in Ca²⁺ and Mg²⁺ free PBS for 10 minutes at room temperature. Culture dishes were then tapped on the lab bench and a large portion of unfused, mononucleated RAW cells were released, and these released cells were collected via centrifugation. This process was repeated 2-4 times until dishes were left with a population of primarily fused, multinucleated syncytia. Mononucleated and multinucleated cell fractions were then processed for biochemical or imaging experiments as described below.

Human Osteoblast/Osteoclast Co-culture: Osteoblasts isolated from the trabecular bone of healthy individuals were obtained from PromoCell (#C12720) and cultured according to the manufacturer's instructions. Osteoclast precursors were derived from primary human monocytes by 6 days of culture in M-CSF as described above. Osteoblasts and osteoclast precursors were cultured in 35 mm dishes with 4-well culture inserts (ibidi, #81156) at a 3:1 well ratio. 48 hours before co-culture mixing, osteoblasts were switched to serum free alpha MEM with 1×pen/strep (Gibco). Following serum starvation, 4-well culture inserts were removed, and cells were cultured in their conditioned media overnight with or without treatment. Cells were fixed with 4% paraformaldehyde the following morning.

HA0-expressing cells and RBCs: NIH 3T3 mouse fibroblasts of clone 15 cell line that stably express influenza were used. These HA0-expressing cells were cultured at 37° C. and 5% CO₂ in DMEM supplemented with 10% heat-inactivated FBS and antibiotics. The cells were used without trypsin pretreatment to keep HA in a fusion-incompetent form. Human red blood cells (RBCs) were isolated from the blood of healthy anonymous donors. RBC were labeled with the fluorescent membrane dye PKH26 and loaded with the fluorescent water-soluble dye CF, as described in (77).

Constructs & Recombinant proteins & Transfection: Recombinant La 1-408, 1-375, 1-375 Q20A_Y24A_D33I, 1-187 and 188-375 were amplified with primers designed with overlapping sequences and inserted between NdeI/HindIII in the V78 pET28A E. coli expression vector, adding to each a N-terminal 6×his affinity tag. La 1-408 was expressed as a recombinant protein in E. coli and purified using IMAC columns by SD Biosciences (San Diego, CA). The remaining constructs were transformed into BL2 (DE3) chemically competent E. coli (Thermo Fisher Scientific) and protein expression was induced with IPTG. Cells were lysed with Bugbuster (Sigma), and 6×his-La proteins were affinity purified using HisPur Cobalt Spin columns (Thermo Fisher Scientific), each according to the manufacturer's instructions. Endotoxin contaminates were depleted from affinity purified 6×his-La proteins using Pierce high-capacity endotoxin removal columns (Thermo Fisher Scientific), according to the manufacturer's instructions. Proteins were then sterile filtered, aliquoted and kept at −80 C.

Plasmids were introduced into primary osteoclasts at day 2 of RANKL stimulation via jetPRIME (Polyplus Transfection). FLAG-La 1-408, FLAG-La 1-375 and FLAG-La 1-375 Q20A_Y24A_D33I plasmids were a gift of the Maraia Lab (NICHD). Briefly, SSB (UniProt P05455) was inserted between HindIII and BamHI the pFLAG-CMV2 vector (Sigma). “Uncleavable” La was produced by taking the FLAG-La 1-408 plasmid and making two point mutations at amino acids D371A and D374A, abrogating the caspase cleavage sites at the C-terminal region of the protein (Emory Integrated Genomics Core). siRNA were introduced into primary osteoclasts after 1 day of RANKL stimulation via Lipofectamine RNAiMAX (Thermo Fisher Scientific). Non-targeted (Cat #4390843) and SSB-targeted (Cat #4392420_ID:s13469) siRNA were introduced at a concentration of 5 ng/ml (Silencer Select, Ambion).

Antibodies: α-Cyclophilin B (CST, D1VdJ), α-GAPDH (CST, D16H11), α-Tubulin (Abcam, 7750), α-RANK (Abcam, 13918), α-Anx A5 (Abcam, 54775), control rabbit polyclonal IgG (Abcam, 27478), IgG2a (Abcam, 18415) used as an isotype control for α-La, Abcam, 75927), IgG1 (Abcam, 170190 used as an isotype control for α-Anx A5, Abcam, 54775), α-Anx A1 (Abcam, 47661), α-Anx A4 (Abcam, 65846), α-6×His (Abcam, 18184) and α-FISH (Abcam, 118575).

The following anti-La antibodies were used for immunoblotting, immunostaining and immunoinhibition where indicated: Abcam, 75927 (referred to α-La); Anti-SSB antibody Invitrogen, PA5-29763 (referred to as α-LMW La); anti-La Phospho-Ser366 Abcam, 61800 (referred to as α-FL La). La antibodies used in the studies were characterized to identify the molecular species of La they recognized in human osteoclasts derived from monocytes, see the Table above. To evaluate the selectivity of these antibodies for the two La species observed during osteoclastogenesis, lysates were probed from differentiating, human osteoclasts at the times when they primarily contained only one of the two La species described (i.e. mostly LMW La or mostly FL La). Abcam 75927 antibody (“α-La”) recognized both La species in Westerns. Invitrogen PA5-29763 (“α-LMW La”) preferentially recognizes LMW La. LMW La is a cleavage product of FL La; thus, FL La contains all the residues in LMW La. The finding that α-LMW La preferentially recognizes LMW La suggests that this antibody must recognize a posttranslational or conformational epitope that differs in LMW vs FL La and not simply the primary amino acid sequence common to both. To specifically recognize FL La, Abcam 61800 antibody to phosphorylated human La (phosphoSer366) was used. While this α-FL La antibody did not work for Western blotting in these experiments, in immunofluorescence staining, this antibody recognized FL La but not LMW La. In differentiating human osteoclasts at intermediate time point (Day 4 post RANKL addition), where Western blot analysis with α-La recognized both of osteoclast La molecular species, α-FL La exclusively recognized nuclear La, while α-LMW primarily recognized La in the cytoplasm.

Biochemical approaches: Cells were lysed on ice via pulse sonication and rotated end over end at 4° C. for 45 minutes in the presence of protease inhibitors (cOmplete, Sigma, #118361700010). Steady-state protein levels were evaluated via SDS-PAGE followed by immunoblotting. Bulk proteins were evaluated via SDS-PAGE followed by silver stain (SilverQuest, Thermo Fisher Scientific). Bands of interest were cut from silver stained gels, destained and evaluated by liquid chromatography coupled with tandem mass spectrometry (Proteomics Core, NHLBI). The selective enrichment of cytosolic vs membrane associated protein fractions was carried out using MEM-PER™ Plus Membrane Protein Extraction Kit (Thermo Fisher Scientific, catalogue #89842) according to the manufacturer's instructions.

Immunoprecipitations were performed as described previously (78). Briefly, multi-protein complexes were sub-stocieometrically crosslinked using the non-membrane permeable, 12A length, cleavable crosslinker 3,3′ Dithiobis(sulfosuccinimidylpropionate) (DTSSP) according to maufacture's instructions (Thermo Fisher Scientific). Supermolecular complexes were immunoprecipitated using Sheep α-Ms IgG magnetic Dynabeads (Invitrogen) decorated with Ms antibodies targeting proteins of interest (α-La, Abcam, 75927; α-Anx A5, Abcam, 54775; IgG2a, Abcam, 18415; or IgG1, Abcam, 170190). Supramolecular complexes were denatured, crosslinking was cleaved via addition of reducing reagents (BME, BioRad), and proteins within these complexes were separated via PAGE. Proteins were transferred and probed for proteins of interest using immunoblotting (as described above). Rb antibodies were used to probe membranes for proteins of interest (α-La PA5-29763, α-Anx A5 14196, α-Anx A1 47661, or α-Anx A4 65846).

Transcript Analysis: For real-time PCR, total RNA was collected from cell lysates using PURELINK™ RNA kit following the manufacturer's instructions (Invitrogen #12183018A). cDNA was generated from total RNA via reverse transcription reaction using a High-Capacity RNA-to-cDNA kit according to the manufacturer's instructions (Applied Biosystems, #4387406). cDNA was then amplified using the IQ™ SYBR® Green Supermix (Biorad). All primers were predesigned KICQSTART® SYBR® Green primers with the highest rank score specific for the gene of interest or GAPDH control and were used according to the manufacturer's instructions (Sigma). All Real-time PCR reactions were performed and analyzed on a CFX96 real-time system (Biorad), using GAPDH as an internal control. Fold-change of gene expression was determined using the ΔΔCt method. 3-4 independent experiments were performed and each was analyzed in duplicate.

Fusion Assays: Osteoclast fusion in the culture systems was evaluated by fluorescence microscopy (Verma et al., J Biol Chem 293, 254-270, doi:10.1074/jbc.M117.809681 (2018)). Briefly, cells were fixed with 4% paraformaldehyde at timepoints of interest, permeabilized with 0.1% TRITON™ X-100 and blocked with 5% FBS. Cells were then stained with phallodin-ALEXAFLURO®488 and Hoechst to label cells' actin cytoskeleton and nuclei, respectively. 16 randomly selected fields of view were imaged using ALEXAFLURO®488, Hoechst and phase contrast compatible filter sets (BioTek) on a Lionheart FX microscope using a 10×/0.3 NA Plan Fluorite WD objective lens (BioTek) using Gen3.10 software (BioTek). Osteoclast fusion efficiency was evaluated as the number of fusion events between cells in these images, as described previously. Since regardless of the sequence of fusion events, the number of cell-to-cell fusion events required to generate syncytium with N nuclei is always equal to N−1, the fusion number index was calculated as Σ(Nᵢ−1)=Nₜₒₜₐₗ−N_{syn}, where Nᵢ=the number of nuclei in individual syncytia and N_{syn}=the total number of syncytia. The number of fusion events were normalized to the total number of nuclei (including unfused cells) to control for small variations in cell density from dish to dish. In contrast to traditional fusion index measurements, this approach gives equal consideration to fusion between two mononucleated cells, one mononucleated cell and one multinucleated cell and two multinucleated cells. In traditional fusion index calculations, fusion between two multinucleated cells does not change the percentage of nuclei in syncytia. If instead one counts the number of syncytia, a fusion event between two multinucleated is not just missed but decreases the number of syncytia. In contrast, the fusion number index is inclusive of all fusion events.

Osteoclast Membrane Fusion Synchronization: Osteoclast fusion was synchronized as described in (Verma, J Biol Chem 293, 254-270, doi:10.1074/jbc.M117.809681 (2018)). Briefly, osteoclast media was refreshed with 100 ng/ml M-CSF, 100 ng/ml RANKL and 350 m lauroyl-LPC 72 hours post RANKL treatment. Following 16 hours, LPC was removed via 5 washes with fresh media and cells were allowed to fuse in the presence or absence of antibody treatment or recombinant La for 90 minutes.

HA0-RBC fusion assay: To test whether La is capable of mediating fusion, this protein was applied to HA0-expressing cells with RBCs tightly bound by the interactions between sialic acid receptors at the surface of RBCs and HA1 subunit of HA0 (82). HA0, an uncleaved form of HA, mediates binding but does not mediate fusion. HA-cells were twice washed with PBS and incubated for 10 min with a 1 ml suspension of RBCs (0.05% hematocrit). HA-cells were washed with zero to two bound RBC per cell with PBS to remove unbound RBC. Then the cells were exposed to 40 nM FL La. Fusogenic activity (content mixing and/or lipid mixing) was assayed by fluorescence microscopy 1 h after La application.

Liposome binding assay: Multilamellar liposomes were formed from pure PC or 9:1 (w/w) mixture of PC and PS. Both lipid compositions were supplemented with 0.5 mol % of lissamine rhodamine phosphatidylethanolamine. To prepare liposomes, lipid stock in benzene/methanol (95:5) was frozen in liquid nitrogen and freeze-dried overnight using SPEEDVAC™ (Savant). Dried lipid was resuspended in aqueous buffer (100 mM NaCl, 10 mM Hepes, pH 7.0) at 1 mM total lipid concentration and vortexed. Proteins and CaCl₂ were added to the liposomes and the mixtures were incubated on ice for 30 minutes. To pellet, liposomes were centrifugated at 15,000 g for 20 minutes to pellet, based on rhodamine fluorescence ˜95% of liposomes. Centrifugated samples were then fractionated into a top, liposome-depleted fraction and a bottom fraction containing liposomes and liposome-bound proteins. Fractions were then solubilized via addition of Laemmli buffer (Bio Rad) and separated via SDS-PAGE, as described above. Recombinant La and recombinant Anx A5 were detected via their n-terminal 6×His tag via α-6×His antibody (Abcam) and signals for soluble vs liposome bound protein fractions were evaluated via densitometry. Data were presented as a percentage of protein signal bound to liposomes where:

$$Liposome Bound Protein = \frac{Liposome Fraction - Soluble Fraction}{Liposome Fraction + Soluble Fraction} .$$

Fluorescence Microscopy Imaging: In the immunofluorescence experiments the cells were washed with PBS and fixed with warm freshly prepared 4% formaldehyde in PBS (Sigma, F1268) at 37° C. The cells were washed three times with PBS. To permeabilize the cells, they were incubated for 5 min in 0.1% TRITON™ X100 in PBS. The cells were again washed three times with PBS and placed into PBS with 10% FBS, for 10 min at the room temperature to suppress non-specific binding. Then the cells were incubated with primary antibodies for 1 hour in PBS with 10% FBS. After 5 washes in PBS, the cells were placed, for 1 h at room temperature, in PBS with 10% FBS with secondary antibodies (either Anti-rabbit IgG Fab2 ALEXAFLUOR® or Anti-mouse IgG Fab2 ALEXAFLUOR® 488, both Cell Signaling Technology, Catalogue #647 4414S and #4408S, respectively, in 1:500 dilution) and then again washed the cells 5 times with PBS.

In the experiments that required immunostaining of non-permeabilized cells (FIG. 3B, 3C), the cells were fixed as above and then incubated with primary antibodies (10 mg/ml) for 10 min at 37° C. After 2 washes with the full medium and 3 washes with PBS, cells were fixed, as described above. After fixing, the cells were washed 3 times with PBS and placed into PBS with 10% FBS, for 10 min at the room temperature to suppress non-specific binding. Then the cells were incubated with secondary antibodies, as described above (1 hour in PBS with 10% FBS at room temperature) and, finally, washed 5 times with PBS. Images were captured on a Zeiss LSM 800 airyscan, confocal microscope using a C-Apochromat 63×/1.2 water immersion objective.

Bone Resorption: Bone resorption was evaluated using bone resorption assay kits from Cosmo Bio USA according to the manufacturer's instructions. Briefly, fluoresceinamine-labeled chondroitin sulfate was used to label 24-well, calcium phosphate-coated plates. Human, monocyte-derived osteoclasts were differentiated as described above, using alpha MEM without phenol red. Media were collected at 4-5 days post RANKL addition, and fluorescence intensity within the media was evaluated as recommended by the manufacture.

Statistical analysis: Each graph presents data from three separate biological replicates repeated on independent occasions unless stated otherwise in the legend. Data were assembled and analyzed using GraphPad Prism 8.0. For each experiment, cells from the same passage, donor or animal were paired across the differing conditions described. All functional dependencies reported were observed in each independent experiment. However, as known for the human monocyte-derived osteoclasts (Moller et al., Int J Mol Sci 21, doi:10.3390/ijms21176368 (2020)), times course of osteoclastogenic differentiation and baseline extents of fusion considerably varied for monocytes from different donors. Statistical significance was analyzed using a paired ratio t test, where raw values are logarithmically transformed and then assessed. In the analysis of the HA0-RBC experiments, Wilson method-based confidence limits for binominal proportion were calculated in R (v. 4.1.1) using binconf function of Hmisc package (v. 4.5.0).

In view of the many possible embodiments to which the principles of our invention may be applied, it should be recognized that illustrated embodiments are only examples of the invention and should not be considered a limitation on the scope of the invention. Rather, the scope of the invention is defined by the following claims. We therefore claim as our invention all that comes within the scope and spirit of these claims.

1. A method of modulating osteoclast fusion, comprising:
administering an effective amount of a Lupus autoantigen (La) protein or an agent that modulates La protein expression or activity, to a subject in need thereof, thereby modulating osteoclast fusion in the subject.

2. The method of claim 1, wherein the subject is human.

3. The method of claim 1, wherein the administering comprises administering the effective amount of the La protein, or the agent that modulates La protein expression or activity, systemically to the subject.

4. The method of claim 1, wherein the method increases osteoclast fusion and bone resorption in the subject.

5. The method of claim 4, wherein the agent that modulates La protein expression or activity is an agent that increases La protein expression or activity.

6. The method of claim 4, wherein the subject has a disease that comprises reduced bone resorption.

7. The method of claim 6, wherein the disease is osteopetrosis.

8. The method of claim 4, wherein the subject has a bone fracture.

9. The method of claim 1, comprising administering the La protein to the subject.

10. The method of claim 9, wherein the La protein comprises:
a) amino acids 300-375 of SEQ ID NO: 1;
b) amino acids 6-242 of SEQ ID NO: 1, and/or
wherein the La protein does not comprise amino acids 376-408 of SEQ ID NO: 1.

11. The method of claim 1, wherein the La protein comprises or consists of:
a) an amino acid sequence at least 95% identical to SEQ ID NO: 2, wherein the La protein is at most 375 amino acids in length and does not comprise amino acids 376-408 of SEQ ID NO: 1;
b) an amino acid sequence at least 95% identical to SEQ ID NO: 2;
c) the amino acid sequence of SEQ ID NO: 2 [amino acids 1-375 of La];
d) the amino acid sequence of SEQ ID NO: 1;
e) an amino acid sequence at least 95% identical to SEQ ID NO: 7;
f) an amino acid sequence at least 95% identical to SEQ ID NO: 7, wherein the La protein does not comprise amino acids 1-187 and 376-408 of SEQ ID NO: 1; or
g) SEQ ID NO: 7.

12. The method of claim 4, wherein the agent that modulates La protein or activity is a nucleic acid molecule encoding the La protein.

13. The method of claim 12, wherein the La protein comprises:
a) amino acids 300-375 of SEQ ID NO: 1; and/or
b) amino acids 6-242 of SEQ ID NO: 1;
wherein the La protein does not comprise amino acids 376-408 of SEQ ID NO: 1.

14. The method of claim 12, wherein the La protein comprises or consists of:
a) an amino acid sequence at least 95% identical to SEQ ID NO: 2, wherein the La protein is at most 375 amino acids in length and does not comprise amino acids 376-408 of SEQ ID NO: 1;
b) an amino acid sequence at least 95% identical to SEQ ID NO: 2;
c) the amino acid sequence of SEQ ID NO: 2 [amino acids 1-375 of La];
d) the amino acid sequence of SEQ ID NO: 1;
e) an amino acid sequence at least 95% identical to SEQ ID NO: 7;
f) an amino acid sequence at least 95% identical to SEQ ID NO: 7, wherein the La protein does not comprise amino acids 1-187 and 376-408 of SEQ ID NO: 1; or
g) SEQ ID NO: 7.

15. The method of claim 12, wherein the nucleic acid molecule comprises SEQ ID NO: 3.

16. The method of claim 12, comprising administering to the subject an expression vector comprising the nucleic acid molecule encoding the La protein.

17. The method of claim 16, wherein the vector is an adenovirus vector, a lentiviral vector, or an adeno-associated viral vector.

18. The method of claim 1, wherein the method decreases osteoclast fusion and bone resorption in the subject, and wherein the agent decreases La protein expression or activity in the subject.

19. The method of claim 18, wherein the subject has a disease that comprises increased bone resorption.

20. The method of claim 19, wherein the disease is osteoporosis, Paget's disease of bone, fibrous dysplasia, rheumatoid arthritis, osteoclast-rich osteopetrosis, osteomyelitis or metastatic bone disease.

21. The method of claim 18, wherein the agent is an inhibitory nucleic acid molecule, a nucleic acid molecule encoding an inhibitory La peptide, or a CRISPR/Cas system.

22. The method of claim 21, wherein the inhibitory nucleic acid molecule is a small inhibitory (si)RNA, an antisense RNA or a ribozyme.

23. The method of claim 21, wherein the agent is an siRNA comprising or consisting of SEQ ID NO: 4 of SEQ ID NO: 5.

24. The method of claim 18, wherein the agent is an antagonistic antibody that specifically binds the La protein.

25. The method of claim 18, wherein the agent is a caspase inhibitor.

26. The method of claim 18, wherein the agent is an inhibitory peptide, a nucleic acid molecule encoding the inhibitory peptide, or a vector comprising the nucleic acid molecule encoding the inhibitory peptide.

27. The method of claim 26, wherein the inhibitory peptide:
a) consists of SEQ ID NO: 8 or SEQ ID NO: 9
b) comprises SEQ ID NO: 8 or SEQ ID NO: 9 and is at most 35 amino acids in length;
c) consists of SEQ ID NO: 8 or SEQ ID NO: 9 with 1, 2, 3, 4 or 5 conservative substitutions; or
d) comprises SEQ ID NO: 8 or SEQ ID NO: 9 with 1, 2, 3, 4 or 5 conservative substitutions and is at most 35 amino acids in length.

28. The method of claim 26, wherein the agent is the nucleic acid molecule encoding the inhibitory peptide, and wherein the nucleic acid molecule comprises SEQ ID NO: 10 or SEQ ID NO: 11.

29. The method of claim 19, wherein the subject has osteoporosis, and wherein the method further comprises administering to the subject an effective amount of one or more of a bisphosphonate, an antibody that specifically binds Receptor activator of nuclear factor kappa-B ligand (RANKL), or a teriparatide.

30. The method of claim 29, wherein the antibody that specifically binds RANKL is denosumab.

31. (canceled)

32. A kit comprising:
a) an agent decreases La protein expression or activity in the subject; and
b) a bisphosphonate, an antibody that specifically binds RANKL, or a teriparatide.

33. The kit of claim 32, wherein the agent that decreases La protein expression or activity in the subject is:
a) an inhibitory nucleic acid molecule;
b) a CRISPR/Cas system;
c) an antagonistic antibody that specifically binds the La protein;
d) a caspase inhibitor;
e) an inhibitory peptide; or
f) a nucleic acid molecule encoding the inhibitory peptide.

34. The kit of claim 32, wherein the agent that decreases La protein expression or activity is the inhibitory peptide, and wherein the inhibitory peptide:
a) consists of SEQ ID NO: 8 or SEQ ID NO: 9
b) comprises SEQ ID NO: 8 or SEQ ID NO: 9 and is at most 35 amino acids in length;
c) consists of SEQ ID NO: 8 or SEQ ID NO: 9 with 1, 2, 3, 4 or 5 conservative substitutions; or
d) comprises SEQ ID NO: 8 or SEQ ID NO: 9 with 1, 2, 3, 4 or 5 conservative substitutions and is at most 35 amino acids in length.

35. A kit comprising:
a) La protein, an effective fragment thereof, or a nucleic acid molecule or vector encoding the La protein or effective fragment thereof, and
b) latent membrane protein (LMP)-1, fibroblast growth factor (FGF)-2, or a bone morphogenic protein (BMP), or a nucleic acid encoding the LMP-1, FGF-2, or BMP.
